(19) United States
(12) Patent Application Publication
(10) Pub. No.: US 2025/0066345 A1
(43) Pub. Date: Feb. 27, 2025

(54) COMPOSITIONS AND METHODS OF USE TO TREAT 12-LIPOXYGENASE (12-LOX) MEDIATED DISEASES

(71) Applicant: VERALOX THERAPEUTICS, Frederick, MD (US)

(72) Inventors: David MALONEY, Point of Rocks, MD (US); Matthew BOXER, New Market, MD (US)

(21) Appl. No.: 18/723,072

(22) PCT Filed: Dec. 16, 2022
(86) PCT No.: PCT/US2022/081779

§ 371 Date: Jun. 21, 2024

Related U.S. Application Data
(60) Provisional application No. 63/381,191, filed on Oct. 27, 2022.
(60) Provisional application No. 63/390,380, filed on Jul. 19, 2022.
(60) Provisional application No. 63/338,111, filed on May 4, 2022.
(60) Provisional application No. 63/293,132, filed on Dec. 23, 2021.

(51) Int. Cl. C07D 417/12; A61K 31/4439; C07D 213/75

(52) U.S. Cl. CPC C07D 417/12; A61K 31/4439; C07D 213/75

(57) ABSTRACT

The present disclosure provides compounds, compositions comprising them, and pharmaceutical uses thereof, for example, compositions suitable for the treatment of 12 lipoxygenase (12-LOX) mediated diseases.

### CROSS-REFERENCE TO RELATED APPLICATIONS

This patent application is an international patent application which claims priority to U.S. Provisional Application Nos. 63/381,191 filed on Oct. 27, 2022, 63/390,380 filed on Jul. 19, 2022, 63/338,111 filed on May 4, 2022, and 63/293,132 filed on Dec. 23, 2021, the disclosures of each of which are incorporated herein in their entirety for all purposes.

### FIELD

The present disclosure provides compounds, compositions comprising them, and pharmaceutical uses thereof, for example, compositions suitable for the treatment of 12 lipoxygenase (12-LOX) mediated diseases. The disclosure further provides pharmaceutical compositions comprising the compounds, uses of the compounds, and compositions for the treatment of, for example, Heparin-Induced Thrombocytopenia (HIT) and Type 1 Diabetes (T1D).

### BACKGROUND

Regulation of platelet function is essential for the prevention and treatment of cardiovascular atherothrombotic events, such as coronary artery disease and stroke. While current antiplatelet drugs effectively inhibit platelet function and prevent thrombotic complications, these treatments often increase the risk of unwanted bleeding. Therefore, there is an unmet need for the identification of novel antiplatelet targets that block unnecessary platelet activation in disease conditions and reverse platelet inhibition with a minimal risk of bleeding. Platelet adhesion and aggregation at the site of vascular injury are essential for maintaining normal hemostasis and preventing blood loss. However, the same processes can also lead to the development of arterial thrombosis and vessel occlusion when the integrity of the vessel wall is compromised by rupture of an atherosclerotic plaque. Excessive platelet activation and aggregation may lead to the formation of occlusive thrombi and result in severe consequences, such as myocardial infarction, ischemic stroke, and pulmonary embolism, which are the predominant causes of morbidity and mortality worldwide. Antiplatelet therapy is considered a gold standard for its effectiveness in preventing aberrant platelet activation and pivotal in the treatment of cardiovascular atherothrombotic events to reduce morbidity and mortality. Currently approved antiplatelet therapies inhibit platelet function by targeting platelet enzymes, receptors, and glycoproteins. Although these therapeutic approaches limit platelet function, they often result in a concomitant increased risk of bleeding. Therefore, a need exists for the identification of novel antiplatelet therapeutic targets that limit bleeding.

12-lipoxygenase (12-LOX) is an enzyme that oxidizes fatty acids, generates proinflammatory metabolites and has been implicated in a myriad of diseases including Heparin-Induced Thrombocytopenia (HIT) and Type 1 Diabetes (T1D). As such, there is a need to develop therapies for these diseases.

### SUMMARY

Provided herein is a compound of Formula (I)

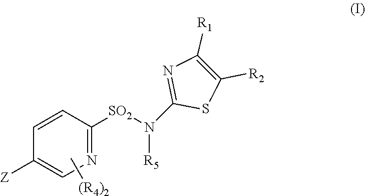

or a pharmaceutically acceptable salt thereof, wherein Z is

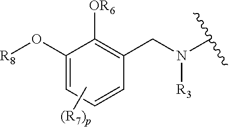

and wherein
R₁ is hydrogen or optionally substituted cycloalkyl, aryl or C₁-C₃ alkylaryl, each of which is optionally substituted by one or more substituents independently selected from halogen, alkyl, haloalkyl, alkyloxy, C₁-C₃ haloalkyloxy, or heterocycle;
R₂ is selected from H, halogen, deuterium, alkyl, haloalkyl, C₁-C₃ alkyloxy and C₁-C₃ haloalkyloxy, hydroxyalkyl, alkyoxyalkyl, cycloalkyl including cyclopropyl, C₁-C₃ alkylaryl, or substituted or unsubstituted aryl and aralkyl including

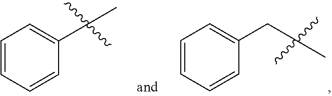 [SMILES: *c1ccccc1 ; *Cc1ccccc1]

or amide including

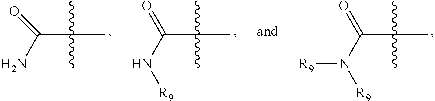

wherein R₉ is independently selected from C₁-C₃ alkyl, cycloalkyl (including cyclopropyl), and substituted or unsubstituted phenyl;
or R₁ and R₂ together form an optionally substituted 5, 6, or 7-membered fused 1,2-carbocyclic ring system with the thiazole ring wherein the ring carbons of the 5, 6, or 7-membered fused ring are optionally substituted by one or more atoms selected from N, O, and S. The fused 5, 6, or 7-membered ring is optionally substituted independently by one or more halogen, C₁-C₃ alkyl, C₁-C₃ haloalkyl, C₁-C₃ alkyloxy, C₁-C₃ haloalkyloxy, or an optionally substituted aryl ring;
or R₁ and R₂ together with the carbons to which they are attached form substituted or unsubstituted

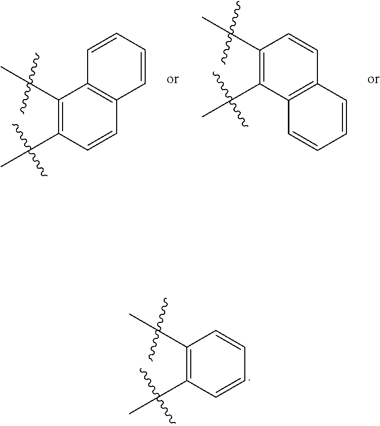 [SMILES: *c1ccc2ccccc2c1* ; *c1ccc2ccccc2c1* ; *c1ccccc1*]

R₃ is hydrogen, C₁-C₃ alkyl, or C₁-C₃ haloalkyl;
each R₄ is independently hydrogen, CD₃, substituted or unsubstituted phenyl, cycloalkyl, including cyclopropyl, C₁-C₃ alkyl, C₁-C₃ haloalkyl, —OR, C₁-C₃ haloalkyloxy, or halogen;
R₅ is hydrogen, C₁-C₃ alkyl, or C₁-C₃ haloalkyl;
R₆ is hydrogen, CD3, alkyl, cycloalkyl, haloalkyl or

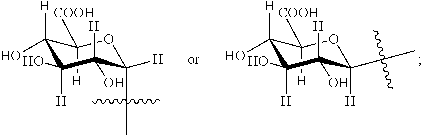

each R₇ is independently hydrogen, alkyl, haloalkyl, cycloalkyl, or halogen;
R₈ is hydrogen, alkyl, haloalkyl, cycloalkyl, or halocycloalkyl;
n is 0, 1, or 2;
p is 0, 1, 2, or 3;
or pharmaceutically acceptable salt, a prodrug, solvate, hydrate, or stereoisomer thereof.

### DETAILED DESCRIPTION

### 1. General Description of Certain Embodiments of the Invention

In one aspect, the present invention provides a compound of formula (I′)

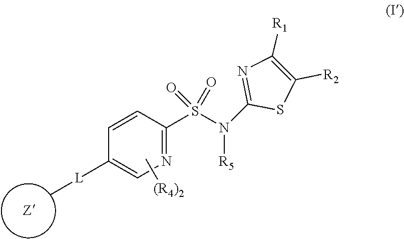

or a pharmaceutically acceptable salt thereof, wherein:
Ring Z′ is an optionally substituted 5-6 membered monocyclic aromatic or heteroaromatic ring having 1-4 heteroatoms independently selected from nitrogen, oxygen or sulfur. One or more optional substitutions in Ring Z′ include replacing one or more of the hydrogen atoms in the C—H bonds of the ring independently with one or more: halogen, C₁-C₆ alkyl, C₁-C₆ haloalkyl, —OR₁₀, —N(R)₂, or

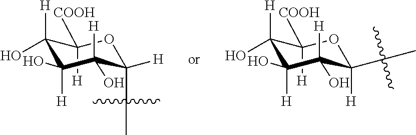

where R₁₀ is independently hydrogen, C₁₋₆ alkyl, C₁₋₆ haloalkyl, C₁₋₆ cycloalkyl, C₁₋₆ halocycloalkyl, phenyl.

L₁ is a covalent bond or a C₁₋₆ bivalent straight or branched hydrocarbon chain wherein one or two methylene units of the chain are independently and optionally replaced by: —O—C(O)—, —C(O)O—, —OC(O)—, —N(R₁₁)—, —C(O)N(R₁₁)—, —(R₁₁)NC(O)—, —OC(O)N(R₁₁)—, —(R₁₁)NC(O)O—, —N(R₁₁)C(O)N(R₁₁)—, —S—, —SO—, —SO₂—, —SO₂N(R₁₁)—, —(R₁₁)NSO2—, —C(S)—, —C(S)O—, —OC(S)—, —C(S)N(R₁₁)—, —(R₁₁)NC(S)—, or —(R₁₁)NC(S)N(R₁₁)—;
wherein R₁₁ is independently hydrogen or an optionally substituted group selected from C₁₋₆ alkyl, C₁₋₆ haloalkyl, C₁₋₆ cycloalkyl, or C₁₋₆ halocycloalkyl or an optionally substituted group selected from C₁₋₆ aliphatic, a 3-8 membered saturated or partially unsaturated monocyclic carbocyclic ring, phenyl, an 8-10 membered bicyclic aromatic carbocyclic ring, a 4-8 membered saturated or partially unsaturated monocyclic heterocyclic ring having 1-2 heteroatoms independently selected from nitrogen, oxygen, or sulfur, a 5-6 membered monocyclic heteroaromatic ring having 1-4 heteroatoms selected from nitrogen, oxygen, or sulfur, or an 8-10 membered bicyclic heteroaromatic ring having 1-5 heteroatoms independently selected from nitrogen, oxygen, or sulfur.

In some embodiments, the present invention provides a compound of formula (I)

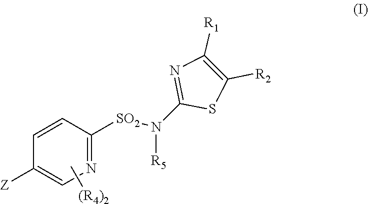

or a pharmaceutically acceptable salt thereof, wherein Z is

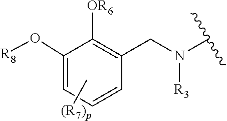

and wherein
R₁ is hydrogen or optionally substituted cycloalkyl, aryl or C₁-C₃ alkylaryl, each of which is substituted by one or more substituents independently selected from halogen, alkyl, haloalkyl, alkyloxy, C₁-C₃ haloalkyloxy and heterocycle;
R₂ is selected from H, halogen, deuterium, alkyl, haloalkyl, C₁-C₃ alkyloxy and C₁-C₃ haloalkyloxy, hydroxyalkyl, alkyoxyalkyl, cycloalkyl including cyclopropyl, C₁-C₃ alkylaryl, or substituted or unsubstituted aryl and aralkyl including

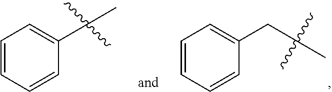 [SMILES: *c1ccccc1 ; *Cc1ccccc1]

or amide including

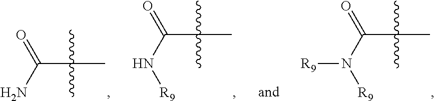

wherein R₉ is independently selected from C₁-C₃ alkyl, cycloalkyl (including cyclopropyl), and substituted or unsubstituted phenyl;
or R₁ and R₂ together form an optionally substituted 5, 6, or 7-membered fused 1,2-carbocyclic ring system with the thiazole ring wherein the ring carbons of the 5, 6, or 7-membered fused ring are optionally substituted by one or more atoms selected from N, O, and S. The fused 5, 6, or 7-membered ring is optionally substituted independently by one or more halogen, C₁-C₃ alkyl, C₁-C₃ haloalkyl, C₁-C₃ alkyloxy, C₁-C₃ haloalkyloxy, or an optionally substituted aryl ring;

or R₁ and R₂ together with the carbons to which they are attached form substituted or unsubstituted or substituted

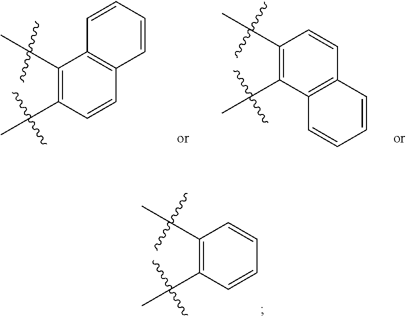 [SMILES: *c1ccc2ccccc2c1* ; *c1ccc2ccccc2c1* ; *c1ccccc1*]

R₃ is hydrogen, C₁-C₃ alkyl, or C₁-C₃ haloalkyl;
each R₄ is independently hydrogen, CD₃, substituted or unsubstituted phenyl, cycloalkyl including cyclopropyl, halogen, C₁-C₃ alkyl, C₁-C₃ alkylaryl, C₁-C₃ haloalkyl, C₁-C₃ alkyloxy, or C₁-C₃ haloalkyloxy;
R₅ is hydrogen, C₁-C₃ alkyl, or C₁-C₃ haloalkyl;
R₆ is hydrogen, CD3, alkyl, cycloalkyl, haloalkyl or

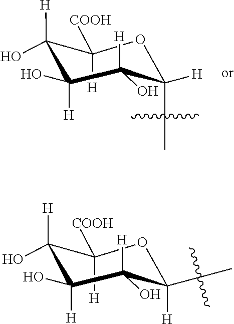

each R₇ is independently hydrogen, halogen alkyl, haloalkyl, or cycloalkyl;
R₈ is hydrogen, alkyl, haloalkyl, cycloalkyl, or halocycloalkyl;
n is 0, 1, or 2;
p is 0, 1, 2, or 3;
or a pharmaceutically acceptable salt, prodrug, solvate, hydrate, or stereoisomer thereof.

Also described herein are methods of treating a disease susceptible to treatment with a disclosed compound in a patient in need thereof by administering to the patient an effective amount of a disclosed compound.

Also described herein are methods of treating a disease or a disorder by administering to a patient in need thereof an effective amount of a disclosed compound.

The disclosure also includes pharmaceutical compositions that comprise an effective amount of a disclosed compound and a pharmaceutically acceptable carrier. The compositions are useful for treating or preventing a disease or disorder. The disclosure includes a disclosed compound provided as a pharmaceutically acceptable prodrug, hydrate, salt, stereoisomer, or mixtures thereof.

The details of the disclosure are set forth in the accompanying description below. Although methods and materials similar or equivalent to those described herein can be used in the practice or testing of the present disclosure, illustrative methods and materials are now described. Other features, objects, and advantages of the disclosure will be apparent from the description and from the claims. In the specification and the appended claims, the singular forms also include the plural unless the context clearly dictates otherwise. Unless defined otherwise, all technical and scientific terms used herein have the same meaning as commonly understood by one of ordinary skill in the art to which this disclosure belongs. All patents and publications cited in this specification are incorporated herein by reference in their entireties.

### 2. Compounds and Definitions

Compounds of the present invention include those described generally herein, and are further illustrated by the classes, subclasses, and species disclosed herein. As used herein, the following definitions shall apply unless otherwise indicated. For purposes of this invention, the chemical elements are identified in accordance with the Periodic Table of the Elements, CAS version, Handbook of Chemistry and Physics, 75^{th} Ed. Additionally, general principles of organic chemistry are described in “Organic Chemistry”, Thomas Sorrell, University Science Books, Sausalito: 1999, and “March's Advanced Organic Chemistry”, 5^{th} Ed., Ed.: Smith, M. B. and March, J., John Wiley & Sons, New York: 2001, the entire contents of which are hereby incorporated by reference.

The articles “a” and “an” are used in this disclosure to refer to one or more than one (i.e., to at least one) of the grammatical object of the article. By way of example, “an element” means one element or more than one element.

The term “and/or” is used in this disclosure to mean either “and” or “or” unless indicated otherwise.

The term “unsaturated”, as used herein, means that a moiety has one or more units of unsaturation.

As used herein, the term “bivalent C_{1 to n} saturated or unsaturated, straight or branched, hydrocarbon chain”, refers to bivalent alkylene, alkenylene, and alkynylene chains that are straight or branched as defined herein.

The term “aliphatic” or “aliphatic group”, as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic that has a single point of attachment to the rest of the molecule. Unless otherwise specified, aliphatic groups contain 1-6 aliphatic carbon atoms. In some embodiments, aliphatic groups contain 1-5 aliphatic carbon atoms. In other embodiments, aliphatic groups contain 1-4 aliphatic carbon atoms. In still other embodiments, aliphatic groups contain 1-3 aliphatic carbon atoms, and in yet other embodiments, aliphatic groups contain 1-2 aliphatic carbon atoms. In some embodiments, “cycloaliphatic” (or “carbocycle” or “cycloalkyl”) refers to a monocyclic C₃-C₆ hydrocarbon that is saturated or that contains one or more units of unsaturation but which is not aromatic, that has a single point of attachment to the rest of the molecule. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

The term, “hydrogen” as used here when referring to an atomic substituent, means a covalent hydrogen atom, as in, for example, H₂O, —CH₃, —NH₂, —OH, (methyl radical) or CH₄. It does not refer to diatomic hydrogen gas (H₂).

The term “alkylene” refers to a bivalent alkyl group. An “alkylene chain” is a polymethylene group, i.e., —(CH₂)ₙ—, wherein n is a positive integer, preferably from 1 to 6, from 1 to 4, from 1 to 3, from 1 to 2, or from 2 to 3. A substituted alkylene chain is a polymethylene group in which one or more methylene hydrogen atoms are replaced with a substituent. Suitable substituents include those described below for a substituted aliphatic group.

The term “alkenylene” refers to a bivalent alkenyl group. A substituted alkenylene chain is a polymethylene group containing at least one double bond in which one or more hydrogen atoms are replaced with a substituent. Suitable substituents include those described below for a substituted aliphatic group.

As used herein, the term “cyclopropylenyl” refers to a bivalent cyclopropyl group of the following structure:

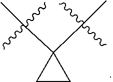 [SMILES: *C1(*)CC1]

The term “halogen” or “halo” refers to a covalently bonded halogen atom, such as, —F, —Cl, —Br, or —I. The term “fluorine” as used herein refers to a covalently bonded fluorine atom as in, for example, CF₃CO₂H, —CF₃. It does not refer to diatomic fluorine gas (F₂) unless specifically stated. As used herein, the same can be said for chlorine, bromine and iodine.

The term “aryl” used alone or as part of a larger moiety as in “aralkyl,” “aralkoxy,” or “aryloxyalkyl,” refers to monocyclic or bicyclic ring systems having a total of five to fourteen ring members, wherein at least one ring in the system is aromatic and wherein each ring in the system contains 3 to 7 ring members. The term “aryl” may be used interchangeably with the term “aryl ring.” In certain embodiments of the present invention, “aryl” refers to an aromatic ring system which includes, but not limited to, phenyl, biphenyl, naphthyl, anthracyl and the like, which may bear one or more substituents. Also included within the scope of the term “aryl,” as it is used herein, is a group in which an aromatic ring is fused to one or more non-aromatic rings, such as indanyl, phthalimidyl, naphthimidyl, phenanthridinyl, or tetrahydronaphthyl, and the like.

The terms “heteroaryl” and “heteroar-,” used alone or as part of a larger moiety, e.g., “heteroaralkyl,” or “heteroaralkoxy,” refer to groups having 5 to 10 ring atoms, preferably 5, 6, or 9 ring atoms; having 6, 10, or 14 π electrons shared in a cyclic array; and having, in addition to carbon atoms, from one to five heteroatoms. The term “heteroatom” refers to nitrogen, oxygen, or sulfur, and includes any oxidized form of nitrogen or sulfur, and any quaternized form of a basic nitrogen. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. The terms “heteroaryl” and “heteroar-”, as used herein, also include groups in which a heteroaromatic ring is fused to one or more aryl, cycloaliphatic, or heterocyclyl rings, where the radical or point of attachment is on the heteroaromatic ring. Nonlimiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3(4H)-one. A heteroaryl group may be mono- or bicyclic. The term “heteroaryl” may be used interchangeably with the terms “heteroaryl ring,” “heteroaryl group,” or “heteroaromatic,” any of which terms include rings that are optionally substituted. The term “heteroaralkyl” refers to an alkyl group substituted by a heteroaryl, wherein the alkyl and heteroaryl portions independently are optionally substituted.

As used herein, the terms “heterocycle,” “heterocyclyl,” “heterocyclic radical,” and “heterocyclic ring” are used interchangeably and refer to a stable 5- to 7-membered monocyclic or 7-10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl), or ⁺NR (as in N-substituted pyrrolidinyl).

A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothiophenyl, pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle,” “heterocyclyl,” “heterocyclyl ring,” “heterocyclic group,” “heterocyclic moiety,” and “heterocyclic radical,” are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl. A heterocyclyl group may be mono- or bicyclic. The term “heterocyclylalkyl” refers to an alkyl group substituted by a heterocyclyl, wherein the alkyl and heterocyclyl portions independently are optionally substituted.

As used herein, the term “partially unsaturated” refers to a ring moiety that includes at least one double or triple bond. The term “partially unsaturated” is intended to encompass rings having multiple sites of unsaturation but is not intended to include aryl or heteroaryl moieties, as herein defined.

As described herein, compounds of the invention may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein.

Each optional substituent on a substitutable carbon is a monovalent substituent independently selected from halogen; —(CH₂)₀₋₄R^{∘}; —(CH₂)₀₋₄OR^{∘}; —O(CH₂)₀₋₄R^{∘}, —O—(CH₂)₀₋₄ C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄ N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄C(O)R^{∘}; —OC(O)(CH₂)₀₋₄SR—, SC(S)SR^{∘}; —(CH₂)₀₋₄SC(O)R^{∘}; —(CH₂)₀₋₄C(O)NR^{∘}₂; —C(S)NR^{∘}₂; —C(S)SR^{∘}; —SC(S)SR^{∘}, —(CH₂)₀₋₄C(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —S(O)(NR^{∘})R^{∘}; —S(O)₂N═C(NR^{∘}₂)₂; —(CH₂)₀₋₄ S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —P(O)₂R^{∘}; —P(O)R^{∘}₂; —OP(O)R^{∘}₂; —OP(O)(OR^{∘})₂; SiR^{∘}₃; —(C₁₋₄ straight or branched alkylene)O—N(R^{∘})₂; or —(C₁₋₄ straight or branched alkylene)C(O)O—N(R^{∘})₂.

Each R^{∘} is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted by a divalent substituent on a saturated carbon atom of R^{∘} selected from ═O and ═S; or each R^{∘} is optionally substituted with a monovalent substituent independently selected from halogen, —(CH₂)₀₋₂R^{●}, —(haloR^{●}), —(CH₂)₀₋₂OH, —(CH₂)₀₋₂OR^{●}, —(CH₂)₀₋₂CH(OR^{●})₂; —O(haloR^{●}), —CN, —N₃, —(CH₂)₀₋₂C(O)R^{●}, —(CH₂)₀₋₂C(O)OH, —(CH₂)₀₋₂C(O)OR^{●}, —(CH₂)₀₋₂SR^{●}, —(CH₂)₀₋₂SH, —(CH₂)₀₋₂NH₂, —(CH₂)₀₋₂NHR^{●}, —(CH₂)₀₋₂NR^{●}₂, —NO₂, —SiR^{●}₃, —OSiR^{●}₃, —C(O)SR^{●}, —(C₁₋₄ straight or branched alkylene)C(O)OR^{●}, or —SSR^{●}.

Each R^{●} is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur, and wherein each R^{●} is unsubstituted or where preceded by halo is substituted only with one or more halogens; or wherein an optional substituent on a saturated carbon is a divalent substituent independently selected from ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, or a divalent substituent bound to vicinal substitutable carbons of an “optionally substituted” group is —O(CR*₂)₂₋₃O—, wherein each independent occurrence of R* is selected from hydrogen, C₁₋₆ aliphatic or an unsubstituted 5-6-membered saturated, partially unsaturated, or aryl ring having 0-4 heteroatoms independently selected from nitrogen, oxygen, or sulfur.

When R* is C₁₋₆ aliphatic, R* is optionally substituted with halogen, —R^{●}, —(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur, and wherein each R^{●} is unsubstituted or where preceded by halo is substituted only with one or more halogens.

An optional substituent on a substitutable nitrogen is independently —R^{†}, —NR^{†}₂, —C(O)R^{†}, —C(O)OR^{†}, —C(O)C(O)R^{T}, —C(O)CH₂C(O)R^{T}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic, unsubstituted —OPh, or an unsubstituted 5-6-membered saturated, partially unsaturated, or aryl ring having 0-4 heteroatoms independently selected from nitrogen, oxygen, or sulfur, or, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur; wherein when R^{†} is C₁₋₆ aliphatic, R^{†} is optionally substituted with halogen, —R^{●}, —(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur, and wherein each R^{●} is unsubstituted or where preceded by halo is substituted only with one or more halogens.

“C₁-C₃ alkyl” refers to a straight or branched chain saturated hydrocarbon containing 1-3 carbon atoms. Examples of a C₁-C₃ alkyl group include, but are not limited to, methyl, ethyl, propyl and isopropyl.

“C₁-C₅ alkyl” refers to a straight or branched chain saturated hydrocarbon containing 1-5 carbon atoms. Examples of a C₁-C₅ alkyl group include, but are not limited to, methyl, ethyl, propyl, butyl, pentyl, isopropyl, isobutyl, sec-butyl, tert-butyl, isopentyl and neopentyl.

Alkyl may be generally lower alkyl, or C₁-C₆ alkyl. Examples of a C₁-C₆ alkyl group include, but are not limited to, methyl, ethyl, propyl, butyl, pentyl, hexyl, isopropyl, isobutyl, sec-butyl, tert-butyl, isopentyl, neopentyl, and isohexyl.

Alkyloxy may be generally lower alkyl, or C₁-C₆ alkyl. Examples of a C₁-C₆ alkyl group include, but are not limited to, methyl, ethyl, propyl, butyl, pentyl, hexyl, isopropyl, isobutyl, sec-butyl, tert-butyl, isopentyl, neopentyl, and isohexyl. Alkyloxy is generally alkyl covalently bonded to oxygen.

Halogen may be F, Cl, Br or I.

Haloalkyl may be generally lower haloalkyl, or C₁-C₆ haloalkyl, or C₁-C₃ alkyl. Examples of C₁-C₃ haloalkyl include —CH₂F, —CHF₂, —CF₃, —CH₂Cl, —CHCl₂, —CCl₃, —CF₂CF₃, —CF₂CF₂H, and —CH₂CF₃.

Haloalkyloxy may be generally haloalkyl, e.g., lower alkyl, that is, C₁-C₆ haloalkyl, or C₁-C₃ haloalkyl, bonded to oxygen. Examples of C₁-C₃ haloalkyl include CH₂F, CHF₂, CF₃, CH₂Cl, CHCl₂, CCl₃, CF₂CF₃, CF₂CF₂H, and CH₂CF₃.

Cycloalkyl means monocyclic saturated carbon rings taken from cyclopropyl, cyclobutyl, cyclopentyl, cyclohexyl, cycloheptanyl and cyclooctanyl.

Heterocycloalkyl means monocyclic saturated carbon rings taken from cyclopropyl, cyclobutyl, cyclopentyl, cyclohexyl, cycloheptanyl and cyclooctanyl wherein a heteroatom, e.g., nitrogen, oxygen, or sulfur, is bonded to two carbons in the ring.

Spirocycle means monocyclic saturated carbon rings taken from cyclopropyl, cyclobutyl, cyclopentyl, cyclohexyl, cycloheptanyl and cyclooctanyl and optionally wherein a heteroatom, e.g., nitrogen, oxygen, or sulfur, is bonded to two carbons in the ring.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

The disclosure also includes pharmaceutical compositions comprising an effective amount of a disclosed compound and a pharmaceutically acceptable carrier. Representative “pharmaceutically acceptable salts” include, e.g., water-soluble and water-insoluble salts, such as the acetate, amsonate (4,4-diaminostilbene-2,2-disulfonate), benzenesultonate, benzonate, bicarbonate, bisulfate, bitartrate, borate, bromide, butyrate, calcium, calcium edetate, camsylate, carbonate, chloride, citrate, clavulariate, dihydrochloride, edetate, edisylate, estolate, esylate, fiunarate, gluceptate, gluconate, glutamate, glycollylarsanilate, hexafluorophosphate, hexylresorcinate, hydrabamine, hydrobromide, hydrochloride, hydroxynaphthoate, iodide, isothionate, lactate, lactobionate, laurate, magnesium, malate, maleate, mandelate, mesylate, methylbromide, methylnitrate, methylsulfate, mucate, napsylate, nitrate, N-methylglucamine ammonium salt, 3-hydroxy-2-naphthoate, oleate, oxalate, palmitate, pamoate (1,1-methene-bis-2-hydroxy-3-naphthoate, einbonate), pantothenate, phosphate/diphosphate, picrate, polygalacturonate, propionate, p-toluenesulfonate, salicylate, stearate, subacetate, succinate, sulfate, sulfosalicylate, suramate, tannate, tartrate, teoclate, tosylate, triethiodide, and valerate salts.

A “subject” is a mammal, e.g., a human, mouse, rat, guinea pig, dog, cat, horse, cow, pig, or non-human primate, such as a monkey, chimpanzee, baboon or rhesus.

An “effective amount” when used in connection with a compound is an amount effective for treating or preventing a disease in a subject as described herein.

The term “carrier”, as used in this disclosure, encompasses carriers, excipients, and diluents and means a material, composition or vehicle, such as a liquid or solid filler, diluent, excipient, solvent or encapsulating material, involved in carrying or transporting a pharmaceutical agent from one organ, or portion of the body, to another organ, or portion of the body of a subject.

The term “treating” with regards to a subject, refers to improving at least one symptom of the subject's disorder. Treating includes curing, improving, or at least partially ameliorating the disorder.

The term “disorder” is used in this disclosure to mean, and is used interchangeably with, the terms disease, condition, or illness, unless otherwise indicated.

The term “administer”, “administering”, or “administration” as used in this disclosure refers to either directly administering a disclosed compound or pharmaceutically acceptable salt of the disclosed compound or a composition to a subject, or administering a prodrug derivative or analog of the compound or pharmaceutically acceptable salt of the compound or composition to the subject, which can form an equivalent amount of active compound within the subject's body.

The term “prodrug,” as used in this disclosure, means a compound which is convertible in vivo by metabolic means (e.g., by hydrolysis, glucuronidation etc.) to a disclosed compound.

### 2.1 Stereoisomers

The compounds of the present disclosure may contain, for example, double bonds, one or more asymmetric carbon atoms, and bonds with a hindered rotation, and therefore, may exist as stereoisomers, such as double-bond isomers (i.e., geometric isomers (E/Z)), enantiomers, diastereomers, and atropoisomers. Accordingly, the scope of the instant disclosure is to be understood to encompass all possible stereoisomers of the illustrated compounds, including the stereoisomerically pure form (for example, geometrically pure, enantiomerically pure, diastereomerically pure, and atropoisomerically pure) and stereoisomeric mixtures (for example, mixtures of geometric isomers, enantiomers, diastereomers, and atropoisomers, or mixture of any of the foregoing) of any chemical structures disclosed herein (in whole or in part), unless the stereochemistry is specifically identified.

If the stereochemistry of a structure or a portion of a structure is not indicated with, for example, bold or dashed lines, the structure or portion of the structure is to be interpreted as encompassing all stereoisomers of it. If the stereochemistry of a structure or a portion of a structure is indicated with, for example, bold or dashed lines, the structure or portion of the structure is to be interpreted as encompassing only the stereoisomer indicated. For example, (1R)-1-methyl-2-(trifluoromethyl)cyclohexane is meant to encompass (1R,2R)-1-methyl-2-(trifluoromethyl)cyclohexane and (1R,2S)-1-methyl-2-(trifluoromethyl)cyclohexane. A bond drawn with a wavy line indicates that both stereoisomers are encompassed. This is not to be confused with a wavy line drawn perpendicular to a bond which indicates the point of attachment of a group to the rest of the molecule.

The term “stereoisomer” or “stereoisomerically pure” compound as used herein refers to one stereoisomer (for example, geometric isomer, enantiomer, diastereomer and atropoisomer) of a compound that is substantially free of other stereoisomers of that compound. For example, a stereoisomerically pure compound having one chiral center will be substantially free of the mirror image enantiomer of the compound and a stereoisomerically pure compound having two chiral centers will be substantially free of the other enantiomer and diastereomers of the compound. A typical stereoisomerically pure compound comprises greater than about 80% by weight of one stereoisomer of the compound and equal or less than about 20% by weight of other stereoisomers of the compound, greater than about 90% by weight of one stereoisomer of the compound and equal or less than about 10% by weight of the other stereoisomers of the compound, greater than about 95% by weight of one stereoisomer of the compound and equal or less than about 5% by weight of the other stereoisomers of the compound, or greater than about 97% by weight of one stereoisomer of the compound and equal or less than about 3% by weight of the other stereoisomers of the compound. This disclosure also encompasses the pharmaceutical compositions comprising stereoisomerically pure forms and the use of stereoisomerically pure forms of any compounds disclosed herein. Further, this disclosure also encompasses pharmaceutical compositions comprising mixtures of stereoisomers of any compounds disclosed herein and the use of said pharmaceutical compositions or mixtures of stereoisomers. These stereoisomers or mixtures thereof may be synthesized in accordance with methods well known in the art and methods disclosed herein. Mixtures of stereoisomers may be resolved using standard techniques, such as chiral columns or chiral resolving agents. See. for example, Jacques et al, Enantiomers, Racemates and Resolutions (Wiley-Interscience, New York, 1981); Wilen et al, Tetrahedron 33:2725; Eliel, Stereochemistry of Carbon

Compounds (McGraw-Hill, NY, 1962); and Wilen, Tables of Resolving Agents and Optical Resolutions, page 268 (Eliel, Ed., Univ. of Notre Dame Press, Notre Dame, IN, 1972).

### 2.2 Tautomers

As known by those skilled in the art, certain compounds disclosed herein may exist in one or more tautomeric forms. As only one specific chemical structure may only be used to represent one tautomeric form, it will be understood that for convenience, referral to a compound of a given structural formula includes all other possible tautomers of said structural formula. For example, the following is illustrative of tautomers of the compounds of Formula I′ where R₅═H:

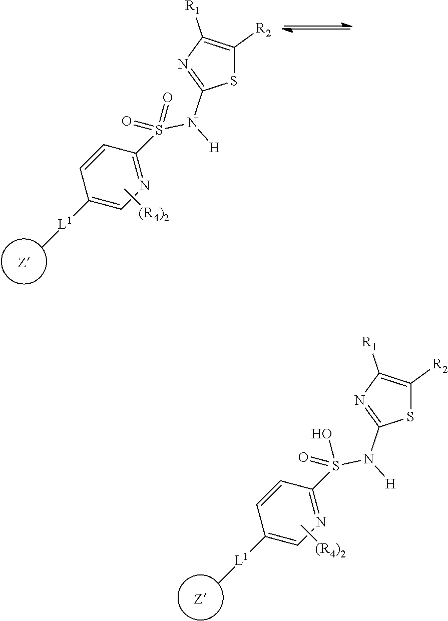

One skilled in the art would recognize that under standard conditions, the tautomer on the left-hand side is clearly the predominant tautomer for these two structures and this example is given for illustrative purposes only. Accordingly, the scope of the instant disclosure is to be understood to encompass all tautomeric forms of the compounds disclosed herein.

### 2.3 Isotopically-Labelled Compounds

Further, the scope of the present disclosure includes all pharmaceutically acceptable isotopically-labelled compounds of the compounds disclosed herein, such as the compounds of Formula I, wherein one or more atoms are replaced by atoms having the same atomic number, but an atomic mass or mass number different from the atomic mass or mass number usually found in nature. Examples of isotopes suitable for inclusion in the compounds disclosed herein include isotopes of hydrogen, such as ²H and ³H, carbon, such as ⁿC, ¹³C and ¹⁴C, chlorine, such as ³⁶Cl, fluorine, such as ¹⁸F, iodine, such as ¹²³I and ¹²⁵I, nitrogen, such as ¹³N and ¹⁵N, oxygen, such as ¹⁵O ¹⁷O and ¹⁸O, phosphorus, such as ³²P, and sulfur, such as ³¹S. Certain isotopically-labelled compounds of Formula I, for example, those incorporating a radioactive isotope, are useful in drug and/or substrate tissue distribution studies. The radioactive isotopes tritium (³H) and carbon-14 (¹⁴C) are particularly useful for this purpose in view of their ease of incorporation and ready means of detection. Substitution with isotopes such as deuterium (²H or D) may afford certain therapeutic advantages resulting from greater metabolic stability, for example, increased in vivo half-life or reduced dosage requirements, and hence may be advantageous in some circumstances. Substitution with positron emitting isotopes, such as ¹¹C, ¹⁸F, ¹⁵O and ¹³N, can be useful in Positron Emission Topography (PET) studies, for example, for examining target occupancy. Isotopically-labelled compounds of the compounds disclosed herein can generally be prepared by conventional techniques known to those skilled in the art or by processes analogous to those described in the accompanying General Synthetic Schemes and Examples using an appropriate isotopically-labelled reagent in place of the non-labelled reagent previously employed.

### 2.4 Solvates

As discussed above, the compounds disclosed herein and the stereoisomers, tautomers, and isotopically-labelled forms thereof or a pharmaceutically acceptable salt of any of the foregoing may exist in solvated or non-solvated forms.

The term “solvate” as used herein refers to a molecular complex comprising a compound or a pharmaceutically acceptable salt thereof as described herein and a stoichiometric or non-stoichiometric amount of one or more pharmaceutically acceptable solvent molecules. If the solvent is water, the solvate is referred to as a “hydrate.”

Accordingly, the scope of the instant disclosure is to be understood to encompass all solvents of the compounds disclosed herein and the stereoisomers, tautomers and isotopically-labelled forms thereof or a pharmaceutically acceptable salt of any of the foregoing.

### 3. Description of Exemplary Embodiments

In some embodiments, the present invention provides a compound of formula (I)

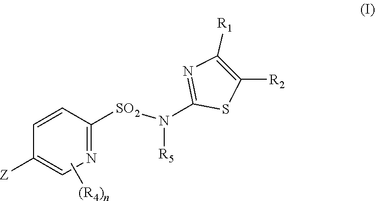

wherein Z is

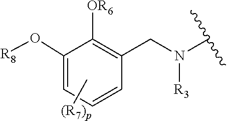

and wherein
R₁ is hydrogen or optionally substituted cycloalkyl, aryl or C₁-C₃ alkylaryl, each of which is optionally substituted by one or more substituents independently selected from halogen, alkyl, haloalkyl, alkyloxy, C₁-C₃ haloalkyloxy or heterocycle;
R₂ is H, halogen, deuterium, alkyl, haloalkyl, C₁-C₃ alkyloxy and C₁-C₃ haloalkyloxy, hydroxyalkyl, alkyoxyalkyl, cycloalkyl including cyclopropyl, C₁-C₃alkylaryl, or substituted or unsubstituted aryl and aralkyl including and

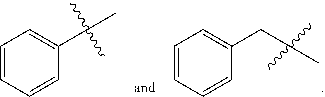 [SMILES: *c1ccccc1 ; *Cc1ccccc1]

or amide including

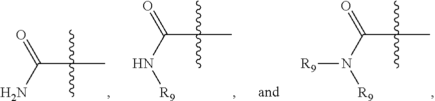

wherein R₉ is independently selected from C₁-C₃ alkyl, cycloalkyl (including cyclopropyl), and substituted or unsubstituted phenyl;
or R₁ and R₂ together form an optionally substituted 5, 6, or 7-membered fused 1,2-carbocyclic ring system with the thiazole ring wherein the ring carbons of the 5, 6, or 7-membered fused ring are optionally substituted by one or more atoms selected from N, O, and S. The fused 5, 6, or 7-membered ring is optionally substituted independently by one or more halogen, C₁-C₃ alkyl, C₁-C₃ haloalkyl, C₁-C₃ alkyloxy, C₁-C₃ haloalkyloxy, or an optionally substituted aryl ring;
or R₁ and R₂ together with the carbons to which they are attached form substituted or unsubstituted or substituted

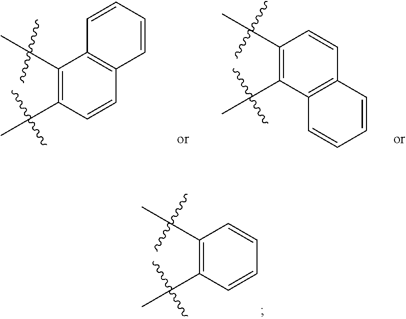 [SMILES: *c1ccc2ccccc2c1* ; *c1ccc2ccccc2c1* ; *c1ccccc1*]

R₃ is hydrogen, C₁-C₃ alkyl, or C₁-C₃ haloalkyl;
each R₄ is independently hydrogen, CD₃, halogen, substituted or unsubstituted phenyl, cycloalkyl including cyclopropyl, C₁-C₃ alkyl, C₁-C₃ alkylaryl, C₁-C₃ haloalkyl, C₁-C₃ alkyloxy, or C₁-C₃ haloalkyloxy;
R₅ is hydrogen, C₁-C₃ alkyl, or C₁-C₃ haloalkyl;
R₆ is hydrogen, CD3, alkyl, cycloalkyl, haloalkyl or

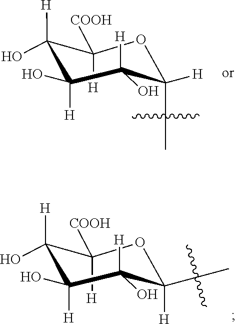

each R₇ is independently hydrogen, halogen alkyl, haloalkyl, or cycloalkyl;
R₈ is hydrogen, alkyl, haloalkyl, cycloalkyl, or halocycloalkyl;
n is 0, 1, or 2;
p is 0, 1, 2, or 3;
or a pharmaceutically acceptable salt, prodrug, solvate, hydrate, or stereoisomer thereof.

As defined generally above, R₁ is optionally substituted aryl or C₁-C₃ alkylaryl which is substituted by one or more substituents independently selected from halogen, alkyl, haloalkyl, alkyloxy, 3-F, 4-oxetanephenyl, and C₁-C₃ haloalkyloxy.

In some embodiments, R₁ is optionally substituted aryl.

In some embodiments, R₁ is

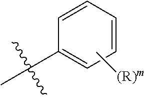

wherein R is independently halogen, C₁₋₃ alkyl, C₁₋₃ haloalkyl, C₁₋₃ cycloalky or C₁₋₃ halocycloalkyl; and wherein m is 0-4.

In some embodiments, R₁ is

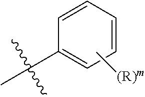

wherein R is independently halogen, C₁₋₃ alkyl, C₁₋₃ haloalkyl, C₁₋₃ cycloalky or C₁₋₃ halocycloalkyl; and wherein m is 1-4.

In some embodiments, R₁ is

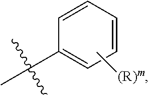

wherein R is independently halogen, C₁₋₃ alkyl, C₁₋₃ haloalkyl, C₁₋₃ cycloalky or C₁₋₃ halocycloalkyl; and wherein m is 1-3.

In some embodiments, R₁ is

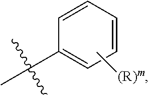

wherein R is independently halogen, C₁₋₃ alkyl, C₁₋₃ haloalkyl, C₁₋₃ cycloalky or C₁₋₃ halocycloalkyl; and wherein m is 1-2.

In some embodiments, R₁ is

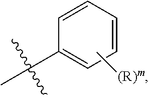

wherein R is independently halogen, C₁₋₃ alkyl, C₁₋₃ haloalkyl, C₁₋₃ cycloalky or C₁₋₃ halocycloalkyl; and wherein m is 1.

In some embodiments, R₁ is

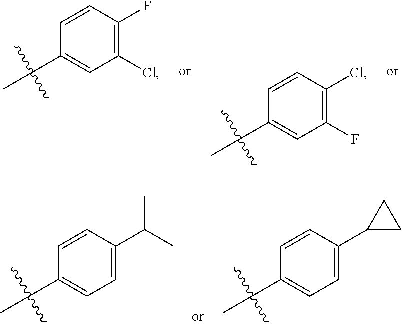

In some embodiments, R₁ is

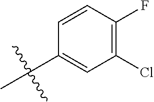 [SMILES: *c1ccc(F)c(Cl)c1]

In some embodiments, R₁ is

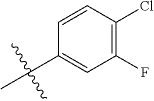 [SMILES: *c1ccc(Cl)c(F)c1]

In some embodiments, R₁ is

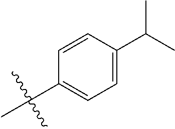 [SMILES: *c1ccc(C(C)C)cc1]

In some embodiments, R₁ is

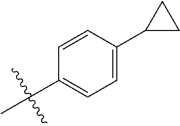 [SMILES: *c1ccc(C2CC2)cc1]

In some embodiments, R₁ is

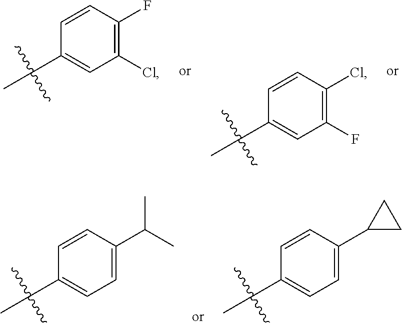

In some embodiments, R₁ is

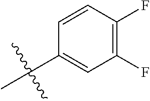 [SMILES: *c1ccc(F)c(F)c1]

In some embodiments, R₁ is

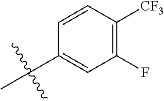 [SMILES: *c1ccc(C(F)(F)F)c(F)c1]

In some embodiments, R₁ is

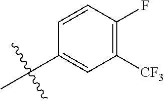 [SMILES: *c1ccc(F)c(C(F)(F)F)c1]

In some embodiments, R₁ is

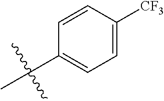 [SMILES: *c1ccc(C(F)(F)F)cc1]

In some embodiments, R₁ is

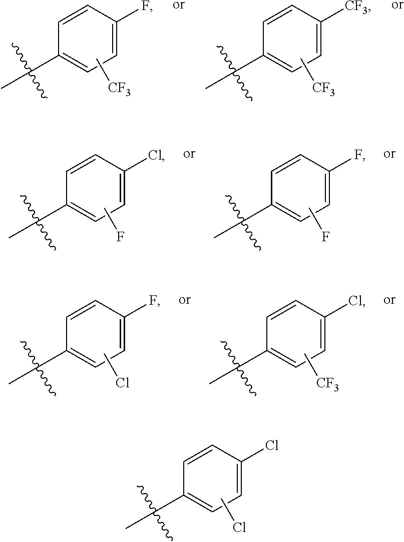

In some embodiments, R₁ is phenyl substituted with a heterocycle, including, without limitation:

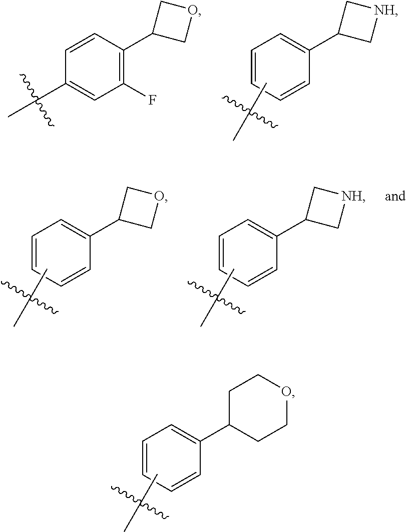

wherein aryl may be optionally substituted with 3-halogen.

In some embodiments, R₁ is selected from the groups in the R₁ position recited in Table 1, below.

As defined generally above, R₂ is hydrogen, halogen, alkyl, cycloalkyl, C₁-C₃ alkylaryl, haloalkyl, C₁-C₃ alkyloxy, C₁-C₃ haloalkyloxy, hydroxyalkyl, or alkyoxyalkyl.

In some embodiments, R₂ is hydrogen or deuterium.

In some embodiments, R₂ is halogen.

In some embodiments, R₂ is F, Cl, Br or I.

In some embodiments, R₂ is F, Cl or Br.

In some embodiments, R₂ is F or Cl.

In some embodiments, R₂ is F.

In some embodiments, R₂ is Cl.

In some embodiments, R₂ is Br.

In some embodiments, R₂ is I.

In some embodiments, R₂ is alkyl.

In some embodiments, R₂ is methyl, ethyl, n-propyl or isopropyl.

In some embodiments, R₂ is methyl.

In some embodiments, R₂ is C₁-C₃ alkylaryl.

In some embodiments, R₂ is methylaryl.

In some embodiments, R₂ is cycloalkyl, including cyclopropyl including

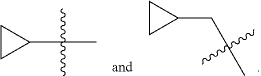

In some embodiments, R₂ is aryl or aralkyl, including

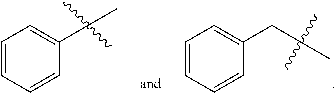 [SMILES: *c1ccccc1 ; *Cc1ccccc1]

In some embodiments, R₂ is cyclopropyl.

In some embodiments, R₂ is cyclobutyl.

In some embodiments, R₂ is cyclopentyl.

In some embodiments, R₂ is cyclopentenyl.

In some embodiments, R₂ is cyclohexyl.

In some embodiments, R₂ is cyclohexenyl.

In some embodiments, R₂ is haloalkyl.

In some embodiments, R₂ is fluoromethyl, fluoroethyl or fluoropropyl.

In some embodiments, R₂ is fluoromethyl.

In some embodiments, R₂ is trifluoromethyl.

In some embodiments, R₂ is C₁-C₃ alkyloxy.

In some embodiments, R₂ is an amide, selected from the group consisting of

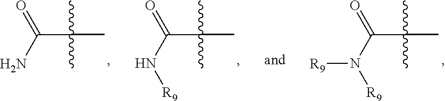

wherein R₉ is independently selected from C₁-C₃ alkyl, cycloalkyl (including cyclopropyl), and substituted or unsubstituted phenyl.

In some embodiments, R₂ is OMe or OEt or OⁱPr.

In some embodiments, R₂ is OMe.

In some embodiments, R₂ is OCF₃.

In some embodiments, R₂ is OCH₂F.

In some embodiments, R₂ is CH₂OH or CH₂CH₂OH or CH₂CH₂CH₂OH or CH₂C(OH)(CH₃)₂.

In some embodiments, R₂ is CH₂OCH₃ or CH₂CH₂OCH₃ or CH₂OCH₂CH₃.

In some embodiments, R₂ is selected from the groups in the R₂ position recited in Table 1, below.

As generally defined above, R₁ and R₂ together form a 5, 6, or 7-membered fused 1,2-carbocyclic ring system with the thiazole ring wherein the ring carbons (not including the 1,2-thiazole-fused ring carbons) of the 5, 6, or 7-membered ring are optionally substituted by one or more ring atoms selected from N, O, and S. Additionally, R₁ and R₂ together may form a fused phenyl ring or a fused naphthalene ring with the thiazole ring. The fused 5, 6, or 7-membered ring is optionally substituted independently by one or more halogen, C₁-C₃ alkyl, C₁-C₃ haloalkyl, C₁-C₃ alkyloxy or C₁-C₃ haloalkyloxy.

In some embodiments, R₁ and R₂ together form a 6-membered ring fused to a thiazole.

In some embodiments, R₁ and R₂ together form a 5-membered ring fused to a thiazole.

In some embodiments, R₁ and R₂ together form a 7-membered ring fused to a thiazole.

In some embodiments, R₁ and R₂ together are optionally substituted

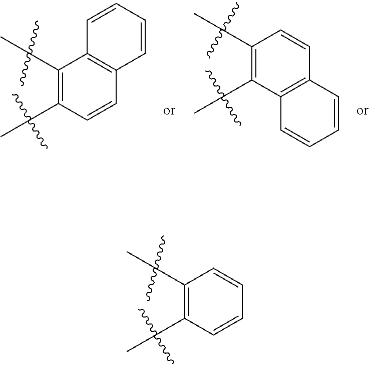 [SMILES: *c1ccc2ccccc2c1* ; *c1ccc2ccccc2c1* ; *c1ccccc1*]

In some embodiments, R₁ and R₂ taken together are optionally substituted

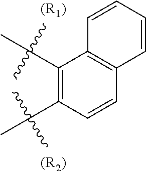

In some embodiments, R₁ and R₂ together are selected from the R₁/R₂ joined groups in the R₁ and R₂ positions depicted in Table 1, below.

As generally described above, R₃ is H, C₁-C₃ alkyl, or C₁-C₃ haloalkyl.

In some embodiments, R₃ is hydrogen.

In some embodiments, R₃ is halogen.

In some embodiments, R₃ is F, Cl, Br or I.

In some embodiments, R₃ is F, Cl or Br.

In some embodiments, R₃ is F or Br.

In some embodiments, R₃ is F or Cl.

In some embodiments, R₃ is Cl or Br.

In some embodiments, R₃ is Cl.

In some embodiments, R₃ is F.

In some embodiments, R₃ is C₁-C₃ alkyl, or C₁-C₃ haloalkyl.

In some embodiments, R₃ is C₁-C₃ alkyl.

In some embodiments, R₃ is C₁-C₃ haloalkyl.

In some embodiments, R₃ is methyl or ethyl.

In some embodiments, R₃ is selected from the groups in the R₃ position recited in Table 1, below.

As generally defined above each R₄ is independently hydrogen, deuterium, CD₃, phenyl, cyclopropyl, halogen, C₁-C₃ alkyl, C₁-C₃ haloalkyl, —C₁-C₃ alkyloxy, C₁-C₃ alkylaryl, C₁-C₃ haloalkyloxy.

In some embodiments, R₄ is independently H.

In some embodiments, R₄ is independently halogen.

In some embodiments, R₄ is independently F, Cl, Br or I.

In some embodiments, R₄ is independently F, Cl or Br.

In some embodiments, R₄ is independently F or Br.

In some embodiments, R₄ is independently F or Cl.

In some embodiments, R₄ is independently Cl or Br.

In some embodiments, R₄ is independently Cl.

In some embodiments, R₄ is independently F.

In some embodiments, R₄ is independently Br.

In some embodiments, R₄ is independently I.

In some embodiments, R₄ is independently C₁-C₃ alkyl.

In some embodiments, R₄ is independently methyl, ethyl, n-propyl or is independently isopropyl.

In some embodiments, R₄ is independently methyl.

In some embodiments, R₄ is independently C₁-C₃ alkylaryl.

In some embodiments, R₄ is independently CH₂-aryl.

In some embodiments, R₄ is independently haloalkyl.

In some embodiments, R₄ is independently fluoromethyl, fluoroethyl or fluoropropyl.

In some embodiments, R₄ is independently fluoromethyl.

In some embodiments, R₄ is independently trifluoromethyl.

In some embodiments, R₄ is independently C₁-C₃ alkyloxy.

In some embodiments, R₄ is independently OMe or OEt or OⁱPr.

In some embodiments, R₄ is independently OMe.

In some embodiments, R₄ is independently OCF₃.

In some embodiments, R₄ is independently OCH₂F.

In some embodiments, R₄ is independently CH₂OH or CH₂CH₂OH or CH₂CH₂CH₂OH or CH₂C(OH)(CH₃)₂.

In some embodiments, R₄ is independently CH₂OCH₃ or CH₂CH₂OCH₃ or CH₂OCH₂CH₃.

In some embodiments, R₄ is independently selected from the groups in the R₄ position recited in Table 1, below.

As generally defined above R₅ is hydrogen, C₁-C₃ alkyl, or C₁-C₃ haloalkyl.

In some embodiments, R₅ is H.

In some embodiments, R₅ is C₁-C₃ alkyl, or C₁-C₃ haloalkyl.

In some embodiments, R₅ is C₁-C₃ alkyl.

In some embodiments, R₅ is methyl, ethyl or propyl.

In some embodiments, R₅ is methyl or ethyl.

In some embodiments, R₅ is methyl.

In some embodiments, R₅ is ethyl or propyl.

In some embodiments, R₅ is ethyl.

In some embodiments, R₅ is propyl.

In some embodiments, R₅ is n-propyl.

In some embodiments, R₅ is isopropyl.

In some embodiments, R₅ is C₁-C₃ haloalkyl.

In some embodiments, R₅ is fluoromethyl, fluoroethyl or fluoropropyl.

In some embodiments, R₅ is fluoromethyl.

In some embodiments, R₅ is trifluoromethyl.

In some embodiments, R₅ is selected from the groups in the R₅ position recited in Table 1, below.

As generally described above, R₆ is hydrogen, CD₃, alkyl, cycloalkyl, haloalkyl or

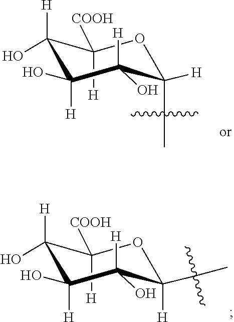

In some embodiments, R₆ is hydrogen.

In some embodiments, R₆ is —CD₃.

In some embodiments, R₆ is C₁-C₆ alkyl, or C₁-C₆ haloalkyl.

In some embodiments, R₆ is C₁-C₃ alkyl, or C₁-C₃ haloalkyl.

In some embodiments, R₆ is C₁-C₃ alkyl.

In some embodiments, R₅ is methyl, ethyl or propyl.

In some embodiments, R₆ is methyl or ethyl.

In some embodiments, R₆ is methyl.

In some embodiments, R₆ is ethyl or propyl.

In some embodiments, R₆ is ethyl.

In some embodiments, R₆ is propyl.

In some embodiments, R₆ is n-propyl.

In some embodiments, R₆ is isopropyl.

In some embodiments, R₆ is C₁-C₃ haloalkyl.

In some embodiments, R₆ is fluoromethyl, fluoroethyl or fluoropropyl.

In some embodiments, R₆ is fluoromethyl.

In some embodiments, R₆ is trifluoromethyl.

In some embodiments, R₆ is C₁-C₆ cycloalkyl

In some embodiments, R₆ is selected from the groups in the R₆ position recited in Table 1, below.

As generally described above, each R₇ is independently hydrogen, alkyl, haloalkyl, cycloalkyl, or halogen.

In some embodiments, each R₇ is independently hydrogen.

In some embodiments, R₇ is H.

In some embodiments, R₇ is C₁-C₆ alkyl, or C₁-C₆ haloalkyl.

In some embodiments, R₇ is C₁-C₃ alkyl, or C₁-C₃ haloalkyl.

In some embodiments, R₇ is C₁-C₃ alkyl.

In some embodiments, R₅ is methyl, ethyl or propyl.

In some embodiments, R₇ is methyl or ethyl.

In some embodiments, R₇ is methyl.

In some embodiments, R₇ is ethyl or propyl.

In some embodiments, R₇ is ethyl.

In some embodiments, R₇ is propyl.

In some embodiments, R₇ is n-propyl.

In some embodiments, R₇ is isopropyl.

In some embodiments, R₇ is C₁-C₃ haloalkyl.

In some embodiments, R₇ is fluoromethyl, fluoroethyl or fluoropropyl.

In some embodiments, R₇ is fluoromethyl.

In some embodiments, R₇ is trifluoromethyl.

In some embodiments, R₇ is C₁-C₆ cycloalkyl

In some embodiments, R₇ is selected from the groups in the R₇ position recited in Table 1, below.

As generally described above, R₈ is hydrogen, alkyl, haloalkyl, cycloalkyl, halocycloalkyl.

In some embodiments, R₈ is hydrogen.

In some embodiments, R₈ is C₁-C₆ alkyl, or C₁-C₆ haloalkyl.

In some embodiments, R₈ is C₁-C₃ alkyl, or C₁-C₃ haloalkyl.

In some embodiments, R₈ is C₁-C₃ alkyl.

In some embodiments, R₈ is ethyl or propyl.

In some embodiments, R₈ is methyl or ethyl.

In some embodiments, R₈ is methyl.

In some embodiments, R₈ is ethyl or propyl.

In some embodiments, R₈ is ethyl.

In some embodiments, R₈ is propyl.

In some embodiments, R₈ is n-propyl.

In some embodiments, R₈ is isopropyl.

In some embodiments, R₈ is C₁-C₃ haloalkyl.

In some embodiments, R₈ is fluoromethyl, fluoroethyl or fluoropropyl.

In some embodiments, R₈ is fluoromethyl.

In some embodiments, R₈ is trifluoromethyl.

In some embodiments, R₈ is C₁-C₈ cycloalkyl

In some embodiments, R₈ is selected from the groups in the R₈ position recited in Table 1, below.

As generally described above, n is 0, 1, or 2.

In some embodiments, n is 0 or 1.

In some embodiments, n is 0 or 2.

In some embodiments, n is 1 or 2.

In some embodiments, n is 0.

In some embodiments, n is 1.

In some embodiments, n is 2.

As generally described above, p is 0, 1, 2 or 3.

In some embodiments, p is 0, 1 or 2.

In some embodiments, p is 0, 1 or 3.

In some embodiments, p is 1, 2 or 3.

In some embodiments, p is 0 or 1.

In some embodiments, p is 0 or 2.

In some embodiments, p is 0 or 3.

In some embodiments, p is 1 or 2.

In some embodiments, p is 1 or 3.

In some embodiments, p is 2 or 3.

In some embodiments, p is 0.

In some embodiments, p is 1.

In some embodiments, p is 2.

In some embodiments, p is 3.

### 4. General Methods of Providing the Present Compounds

The compounds described herein may be prepared or isolated in general by synthetic and/or semi-synthetic methods known to those skilled in the art for analogous compounds and by methods described in detail in the examples, herein.

One skilled in the art would recognize that various functional groups present in compounds of the invention such as aliphatic groups, alcohols, carboxylic acids, esters, amides, aldehydes, halogens and nitriles can be interconverted by techniques well known in the art including, but not limited to reduction, oxidation, esterification, hydrolysis, partial oxidation, partial reduction, halogenation, dehydration, partial hydration, and hydration. See, for example, “March's Advanced Organic Chemistry”, 5^{th} Ed., Ed.: Smith, M. B. and March, J., John Wiley & Sons, New York: 2001, the entirety of which is incorporated herein by reference. Such interconversions may require one or more of the aforementioned techniques, and certain methods for synthesizing compounds of the invention are described herein.

Scheme 1 illustrates a general method of preparing compounds of Formula (I). In one aspect, certain compounds of the present invention of Formula (I), or subformulae thereof, are generally prepared according to Scheme 1 set forth below:

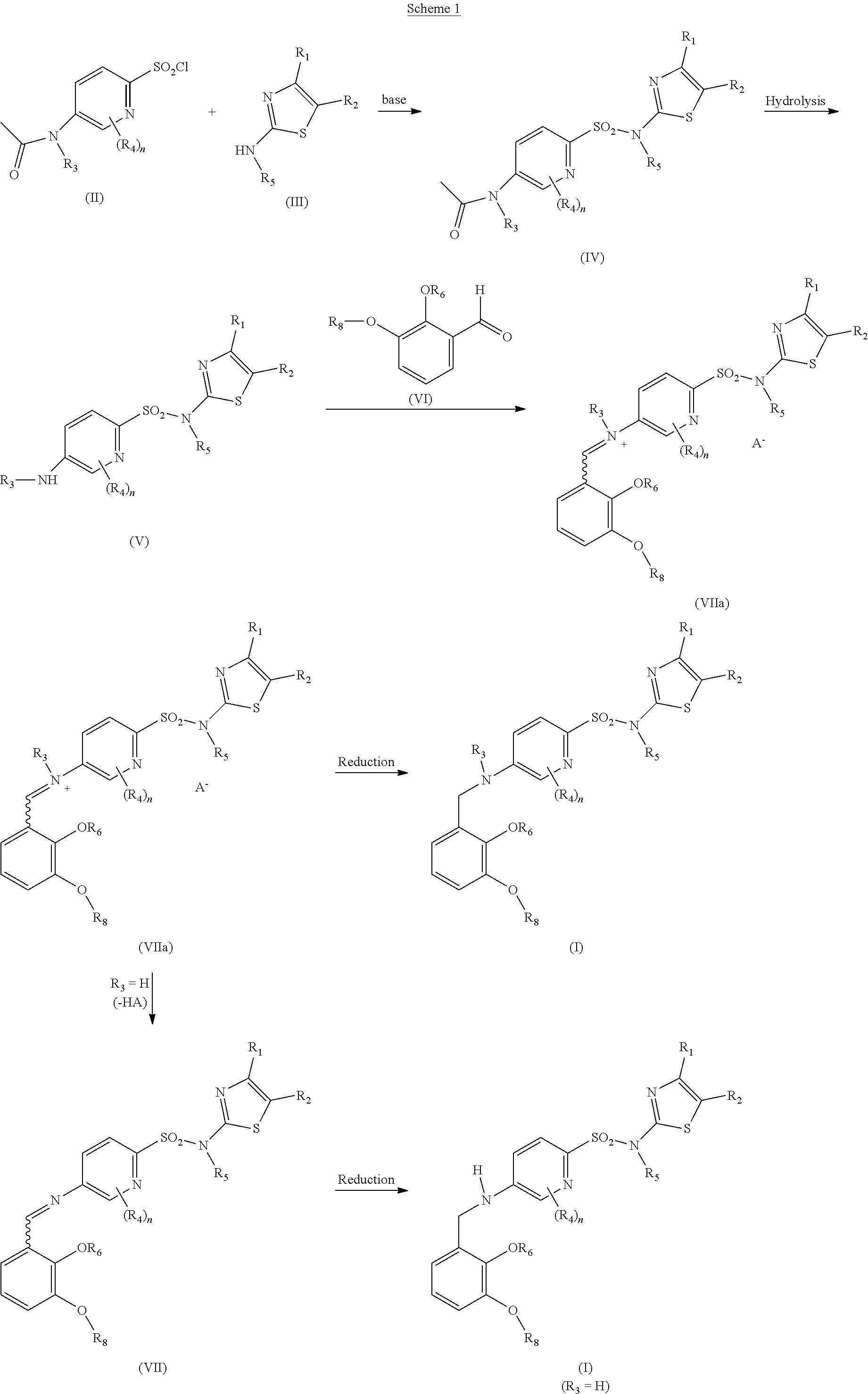

In an embodiment, the compound of formula (II) is

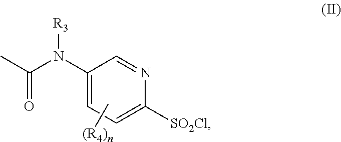

wherein R₃, R₄, and n are defined above, is reacted with a compound of formula (111):

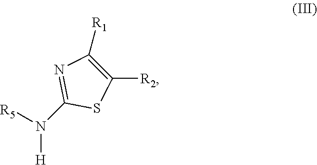

wherein R₁, R₂, and R₅, are defined above, to provide a compound of formula (IV):

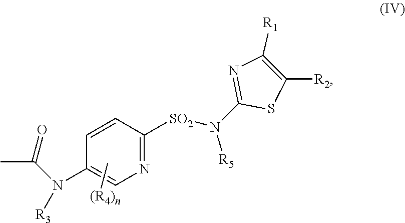

wherein R₁ to R₅ and n are defined above. The compound of formula (IV) is hydrolyzed according to methods known to the person of ordinary skill in the art, for example basic hydrolysis, to provide a compound of formula (V):

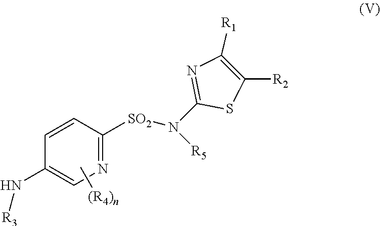

wherein R₁ to R₅ and n are defined above; or a salt thereof.

The compound of formula (V), or a salt thereof, is then reacted with a compound of formula (VI),

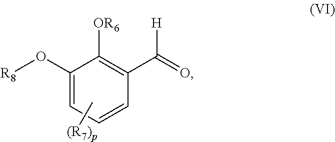

wherein
R₆, R₇, R₈, and p are defined above;
optionally in the presence of a strong acid, to provide a salt of formula (VIIa) or a compound of formula (VII) or a salt thereof:

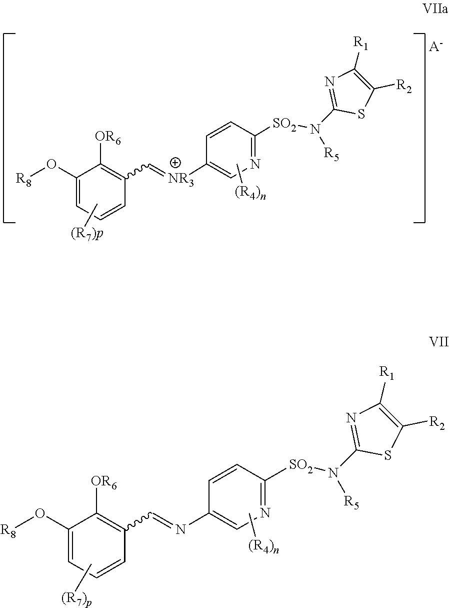

wherein R₁ to R₈, n, and p are defined above, and wherein A is a counterion known to a person of ordinary skill in the art.

The salt of formula (VIIa) and/or compound of formula (VII), or a salt thereof, may be reacted with a reducing agent, e.g., a borohydride such as triacetoxyborohydride, to provide the compound of formula (I).

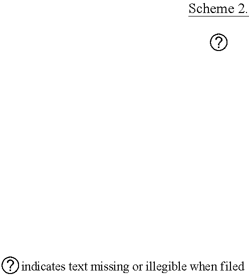

As generally shown in Scheme 1, a starting material compound comprising structure (II) is coupled with a compound of general structure (III) to provide a compound of general structure (IV). Compounds of general structure (IV) may then be deprotected via a hydrolysis reaction affording a compound of general structure (V). Reaction of (V) with aldehyde (VI) affords (VIIa) which may be reduced by a variety of well-known methods to afford compound (I). One skilled in the art would recognize that compound (V) may be converted to compound (I) via well-known reductive amination or other similar methods.

In cases where R₃ is hydrogen in compounds of structure (V), a Schiff base of formula (VII) may be formed and concomitantly or subsequently reduced to afford compound (I″) where R₃ is hydrogen.

### 4.1. Synthesis of Specific Intermediates

### ABBREVIATIONS

The following abbreviations are used herein.
equiv or eq: molar equivalents
o/n: overnight
rt: room temperature
UV: ultra violet
h: hour or hours
HPLC: high pressure liquid chromatography
Rt: retention time
LC-MS: liquid chromatography-mass spectrometry
NMR: nuclear magnetic resonance
CC: column chromatography
TLC: thin layer chromatography
sat: saturated
aq: aqueous
Ac: acetyl
DCM: dichloromethane
DCE: dichloroethane
DEA: diethylamine
DMF: dimethylformamide
DMSO: dimethylsulfoxide
ACN or MeCN: acetonitrile
DIPEA: diisopropylethylamine
EA or EtOAc: ethyl acetate
TEA: triethylamine
THF: tetrahydrofuran
NCS: N-chlorosuccinimide
PE: petroleum ether
Prep.: preparative
TFA: trifluoroacetic acid
FA: formic acid
h: hour(s)
min: minutes
s or sec: second(s)
STAB: sodium triacetoxyborohydride
Cy: cyclohexyl
Tol: toluene

### General Information

All air or moisture sensitive reactions were performed under positive pressure of nitrogen with oven-dried glassware. Chemical reagents and anhydrous solvents were obtained from commercial sources and used as-is. Preparative purification was performed on a Waters semipreparative HPLC. The column used was a Phenomenex Luna C₁₈ (10 μm, 250×75 mm) at a flow rate of 45 mL/min. The mobile phase consisted of acetonitrile and water (each containing 0.1% hydrochloric acid). A gradient of 50-80% acetonitrile over 23 min was used during the purification. Fraction collection was triggered by UV detection (220 nm). Analytical analysis for purity was determined by one method denoted as final QC. Method: Analysis was performed on an Agilent 1260 Infinity series LCMS: LCMS Long Gradient Equivalent 5-100% acetonitrile (0.018% trifluoroacetic acid) in water (0.037% trifluoroacetic acid) over 3 min run time of 4 min with a flow rate of 1 mL/min. A Shim-pack Velox SP-C18 2.7 μm column (3.0×30 mm) was used at a temperature of 50° C. Purity determination was performed using an Agilent diode array detector for method. Mass determination was performed using an Agilent 1260 mass spectrometer with electrospray ionization in the positive mode. 1H and 19F NMR spectra were recorded on Bruker 400 (400) MHz or 600 (376) MHz spectrometers.

Intermediate compounds of formula II, II-1 and II-2

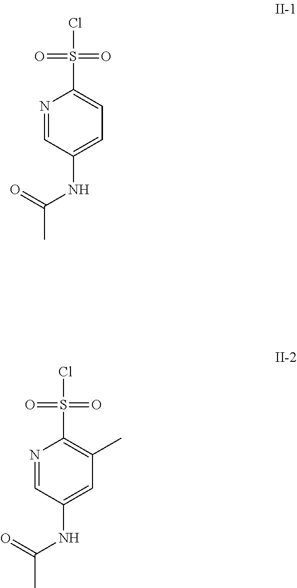

Intermediates II may be reacted with intermediates III to produce compounds of formula (IV).

In another embodiment, compounds of formula II-1 and/or II-2 may be reacted with specific intermediate compounds of formula III, below, to produce the compounds of formula (IV):

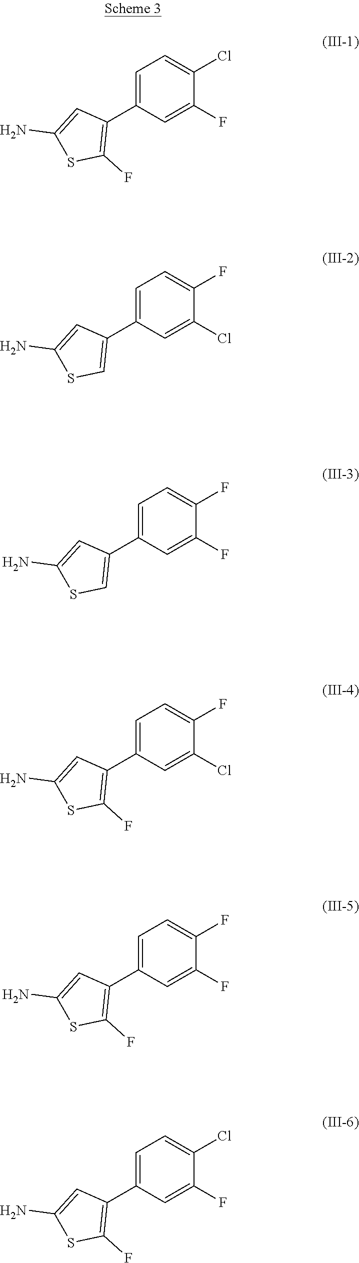 [SMILES: Nc1cc(-c2ccc(Cl)c(F)c2)c(F)s1 ; Nc1cc(-c2ccc(F)c(Cl)c2)cs1 ; Nc1cc(-c2ccc(F)c(F)c2)cs1 ; Nc1cc(-c2ccc(F)c(Cl)c2)c(F)s1 ; Nc1cc(-c2ccc(F)c(F)c2)c(F)s1 ; Nc1cc(-c2ccc(Cl)c(F)c2)c(F)s1]

### Preparation Examples of Intermediates

### Preparation Example 1: Synthesis of 5-acetamidopyridine-2-sulfonyl chloride (II-1, Scheme 4)

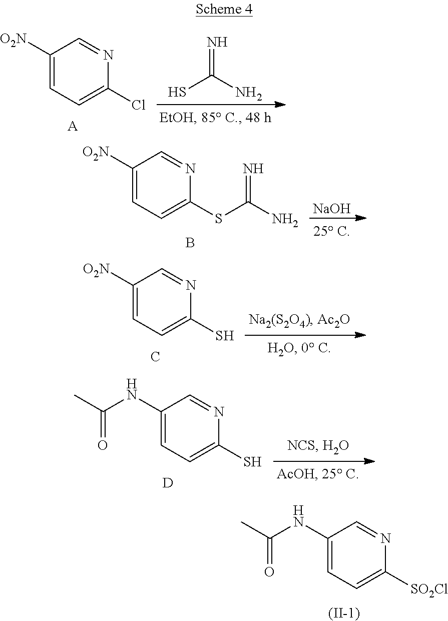

Synthesis of 4-nitro-2-thiopyridine (C). To a mixture of 2-chloro-4-nitropyridine ((A) 8 g, 50.46 mmol, 1 eq) in EtOH (120 mL) was added thiourea (3.99 g, 52.48 mmol, 1.04 eq) in one portion at 25° C. under N₂. The mixture was stirred at 25° C. for 10 min, then heated to 85° C. and stirred for 48 hours. LC-MS showed the starting material was consumed completely. The mixture was cooled to 25° C. and filtered to get crude Compound B. The residue was poured into water (20 mL) treated with 20% NaOH (100 mL). The mixture was stirred for 30 min and filtered. The pH of solution was adjusted to 7 with HCl (2 M), and the precipitated solid collected by filtration to provide Compound C. (3.5 g, 42% yield, 95% purity) was obtained as a red solid. ¹H NMR (400 MHz, CDCl₃) δ 8.48 (d, J=2.5 Hz, 1H), 7.92 (dd, J=9.6, 2.6 Hz, 1H), 7.39 (d, J=9.6 Hz, 1H).

N-(6-mercapto-3-pyridyl)acetamide (D). To a mixture of Compound C (1.58 g, 9.61 mmol, 1 eq) in H₂O (15 mL) was added Na₂(S₂O₄) (5.85 g, 33.62 mmol, 7.32 mL, 3.5 eq) in one portion at 0° C. under N₂. Acetic anhydride (1.37 g, 13.45 mmol, 1.26 mL, 1.4 eq) was then added. The mixture was stirred at 0° C. for 2 hours. LC-MS showed the starting material was consumed completely. A yellow precipitate was filtered, washed with water, and dried to give a yellow solid. (940 mg, 58% yield) was obtained as a yellow solid. ¹H NMR (400 MHz, DMSO) δ 13.35 (s, 1H), 10.07 (s, 1H), 8.22 (d, J=3.8 Hz, 1H), 7.37-7.22 (m, 2H), 2.03 (s, 3H).

5-acetamidopyridine-2-sulfonyl chloride (II-1). To a mixture of Compound D (700 mg, 4.16 mmol, 1 eq) in acetic acid (18 mL) and H₂O (2 mL) was added N-chlorosuccinimide (2.2 g, 16.65 mmol, 4 eq) in one portion at 0° C. under N₂. The mixture was stirred at 25° C. for 4 hours. Thin Layer Chromatography showed the starting material was consumed completely. The mixture was diluted with water (10 ml) and extracted with ethyl acetate (3×10 mL). The combined organic phase was washed with brine (15 mL), dried with anhydrous Na₂SO₄, filtered, and concentrated in vacuum to provide 5-acetamidopyridine-2-sulfonyl chloride (II-1) (800 mg, 78% yield, 95% purity) as an orange solid. ¹H NMR (400 MHz, DMSO) δ 11.30 (s, 1H), 9.07 (s, 1H), 8.52 (d, J=8.8 Hz, 1H), 8.15 (d, J=8.7 Hz, 1H), 2.18 (d, J=11.4 Hz, 3H).

### Preparation Example 2: Synthesis of 5-acetamido-3-methylpyridine-2-sulfonyl chloride (II-2, Scheme 5)

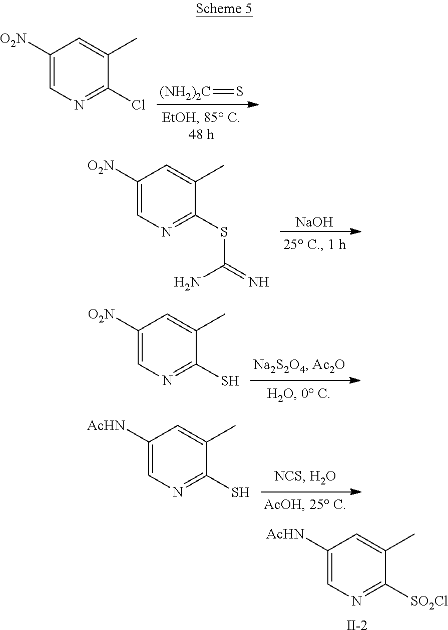

A mixture of thiourea (2.21 g, 28.97 mmol) and 2-chloro-3-methyl-5-nitropyridine (5 g, 28.97 mmol) in EtOH (50 mL) was degassed and purged with dry N₂ 3 times. The mixture was stirred at 85° C. for 4 h under a N₂ atmosphere affording a yellow precipitate. The precipitate was filtered, washed with water (200 mL), and dried to give a yellow solid. Removal of the excess water from the residue under vacuum gave 3-methyl-5-nitropyridin-2-yl carbamimidothioate (4.64 g, 21.9 mmol, 75.5% yield) as a yellow solid.

To a solution of 3-methyl-5-nitropyridin-2-yl carbamimidothioate (4.64 g, 21.86 mmol) in H₂O (9 mL) was added NaOH (54 mL, 20% in H₂O). The mixture was stirred at 25° C. for 1 hour. The reaction mixture was filtered undissolved substance and adjust pH to 7 with HCl (aq., 3M in H₂O), and filtered get to get crude product. Remove the excess water from the filter residue under pressure reduction to give 3-methyl-5-nitropyridine-2-thiol (3 g, 17.6 mmol, 81% yield) as a red solid. LCMS: (M+H)⁺=171.2.

A mixture of 3-methyl-5-nitropyridine-2-thiol (3 g, 17.63 mmol) in H₂O (60 mL) was added Na₂S₂O₄ (10.74 g, 61.7 mmol, 13.4 mL) under N₂, and then added acetyl acetate (2.52 g, 24.7 mmol, 2.31 mL), then the mixture was stirred at 0° C. for 0.5 hour under N₂ atmosphere. The yellow precipitate was filtered, washed with water (100 mL), and dried to give a yellow solid. Removal of the excess water from the filter residue under pressure reduction to give N-(6-mercapto-5-methylpyridin-3-yl)acetamide (1.3 g, 7.13 mmol, 40.5% yield) as a yellow solid.

A solution of N-(6-mercapto-5-methylpyridin-3-yl)acetamide (1.3 g, 7.13 mmol) in AcOH (10 mL) and H₂O (2 mL) was degassed and purged with N₂ 3 times, and NCS (3.33 g, 24.97 mmol) was then added. The mixture was stirred at 0° C. for 0.5 h under N₂ atmosphere. The reaction mixture was diluted with ethyl acetate (10 mL×3), washed with brine (15 mL×2), dried over Na₂SO₄ and concentrated to dryness. The residue was purified by column chromatography (silica gel, 100-200 mesh, 70% ethyl acetate in petroleum ether). 5-acetamido-3-methylpyridine-2-sulfonyl chloride (II-2, 1.1 g, 2.48 mmol, 35% yield) was obtained as a brown solid. LCMS:(M+H)⁺=249.1.

### Preparation Example 3: Synthesis of 4-(4-chloro-3-fluorophenyl)thiazol-2-amine (III-1, Scheme 6)

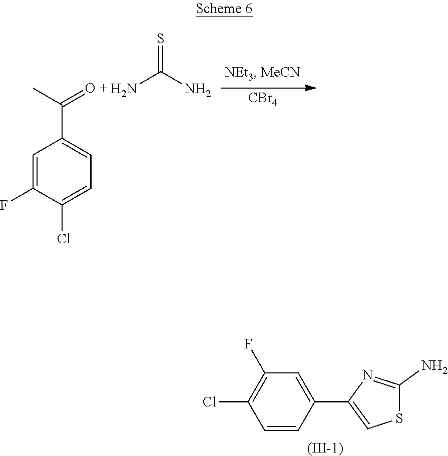

To a mixture of 1-(4-chloro-3-fluorophenyl)ethan-1-one (2.27 g, 13.15 mmol, 1 eq), triethylamine (2.00 g, 19.73 mmol, 2.75 mL, 1.5 eq) and thiourea (2.50 g, 32.88 mmol, 2.5 eq) in MeCN (20 mL) was added carbon tetrabromide (6.54 g, 19.73 mmol, 1.5 eq) in one portion at 25° C. under N₂. The mixture was stirred at 25° C. for 16 hours. LC-MS showed the acetophenone was consumed completely and one new main peak with desired mass was detected. The mixture was concentrated under reduced pressure and the resulting residue poured into water (50 mL) and stirred for 10 min. The aqueous phase was extracted with ethyl acetate (3×20 mL). The combined organic phase was washed with brine (50 mL), dried with anhydrous Na₂SO₄, filtered, and concentrated in vacuo. The residue was purified by silica gel chromatography (petroleum ether/ethyl acetate=1/0, 1/1). to provide 4-(4-chloro-3-fluorophenyl)thiazol-2-amine (III-1) as a yellow solid (2.88 g, 96% yield).

### Preparation Example 4: Synthesis of 4-(3-chloro-4-fluorophenyl)-1,3-thiazol-2-ylamine (III-2, Scheme 7)

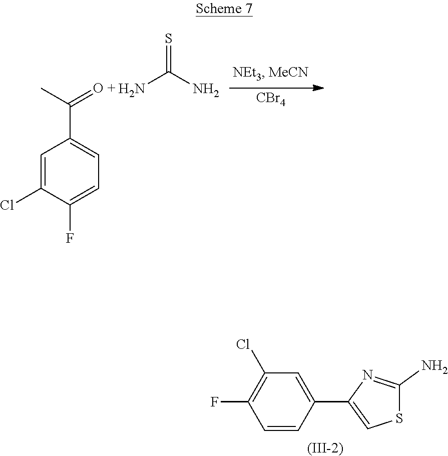

To a mixture of 1-(3-chloro-4-fluorophenyl)ethan-1-one (15 g, 86.91 mmol, 1 eq) and thiourea (9.92 g, 130.37 mmol, 1.5 eq) in MeCN (100 mL) was added CBr₄ (43.23 g, 130.37 mmol, 1.5 eq) and Et₃N (13.19 g, 130.37 mmol, 18.15 mL, 1.5 eq) degassed and purged with N₂ three times. The mixture was stirred at 25° C. for 16 hr. under N₂ atmosphere. LC-MS showed the starting material was consumed completely. The reaction mixture was diluted with 1N HCl (50 mL) and extracted with ethyl acetate (200 mL) and water (200 mL), then was added NH₄Cl and solids were precipitated, and filtered. The filter cake was dried under reduced pressure to provide 4-(3-chloro-4-fluorophenyl)-1,3-thiazol-2-ylamine (III-2) (3.5 g, 14% yield, 80% purity) as a red solid. ¹H NMR (400 MHz, DMSO-d6) 7.96 (dd, J=2.1, 7.3 Hz, 1H), 7.79 (ddd, J=2.2, 4.8, 8.7 Hz, 1H), 7.40 (t, J=9.0 Hz, 1H), 7.14 (s, 3H).

### Preparation Example 5: Synthesis of 4-(3,4-difluorophenyl)-1,3-thiazol-2-amine (III-3, Scheme 8)

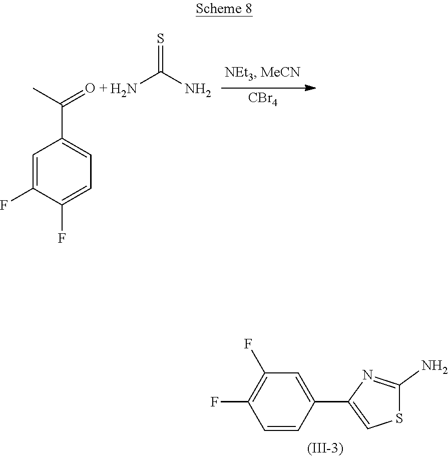

To a mixture of 1-(3,4-difluorophenyl)ethan-1-one (20 g, 128.10 mmol, 16.00 mL) and thiourea (14.63 g, 192.15 mmol) in acetonitrile (300 mL) was added triethylamine (19.44 g, 192.15 mmol, 26.74 mL) and carbon tetrabromide (63.72 g, 192.15 mmol). The mixture was stirred at 25° C. for 16 hours. LC-MS showed the reaction was complete. The reaction mixture was concentrated under reduced pressure to remove the solvent. The resulting residue was triturated with water (200 mL) and extracted with EtOAc (150 mL×3). The combined organic layers were dried over sodium sulfate, filtered and concentrated under reduced pressure to give a residue, which was purified by flash silica gel chromatography (ISCO®; 220 g SepaFlash® Silica Flash Column, Eluent of 22˜30% Ethyl acetate/Petroleum ether gradient @120 mL/min)., Petroleum ether/Ethyl acetate=3:1, Rf=0.3) to yield 4-(3,4-difluorophenyl)-1,3-thiazol-2-amine (III-3, 12.7 g, 58.65 mmol, 6% yield, 98% purity) as a brown solid. LC-MS:(M+H)⁺=212.7; ¹H NMR (400 MHz, DMSO-d₆): 7.78 (ddd, J=1.9, 8.0, 12.4 Hz, 1H), 7.66-7.61 (m, 1H), 7.46-7.36 (m, 1H), 7.11 (br d, J=5.5 Hz, 3H).

### Preparation Example 6: Synthesis of 4-(3-chloro-4-fluorophenyl)-5-fluorothiazol-2-amine (III-4, Scheme 9)

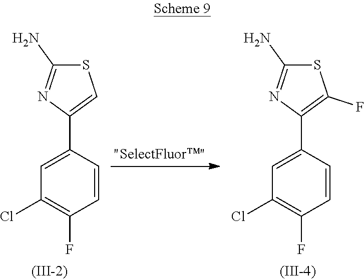 [SMILES: Nc1nc(-c2ccc(F)c(Cl)c2)cs1 ; Nc1nc(-c2ccc(F)c(Cl)c2)c(F)s1 ; "(III-2)" ; "(III-4)"]

To a solution of 4-(3-chloro-4-fluorophenyl)-1,3-thiazol-2-ylamine (III-2,1 g, 4.37 mmol, 1.0 eq) in MeCN (20 mL) was added 1-(chloromethyl) -4-fluoro-1,4-diazoniabicyclo[2.2.2]octane; ditetrafluoroborate (Selectfluor™) (1.55 g, 4.37 mmol, 1.0 eq) in one portion at 0° C. under N₂. The mixture was warmed to 25° C. and stirred for 6 hours. After this time, LC-MS indicated that approximately 12% of the starting material remained. Several new peaks were shown by LC-MS and about 22% of the desired compound (III-4) was detected. Water (30 mL) was added; and the reaction mixture was diluted with EtOAc (30 mL) and water (10 mL). The aqueous layer was extracted with EtOAc (2×20 mL). The combined organic phases were washed with water (2×15 mL) and brine (2×15 mL), dried with anhydrous Na₂SO₄, filtered, and concentrated in vacuum. The residue was purified by silica gel chromatography (SiO₂, PE/EtOAc=0 to 100% EA). 4-(3-chloro-4-fluorophenyl)-5-fluorothiazol-2-amine (III-4) was isolated (230 mg, 21% yield) as a brown solid and used into the next step without further purification. ¹H NMR (400 MHz, DMSO-d6) δ 7.79 (dd, J=7.3, 2.1 Hz, 1H), 7.68 (ddd, J=8.6, 4.7, 2.2 Hz, 1H), 7.48 (t, J=9.0 Hz, 1H), 7.08 (s, 2H).

### Preparation Example 7: Synthesis of 4-(3,4-difluorophenyl)-5-fluorothiazol-2-amine (III-5, Scheme 10)

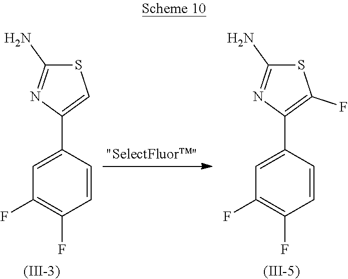 [SMILES: Nc1nc(-c2ccc(F)c(F)c2)cs1 ; Nc1nc(-c2ccc(F)c(F)c2)c(F)s1 ; "(III-3)" ; "(III-5)"]

To a solution of 4-(3,4-difluorophenyl)-1,3-thiazol-2-amine (III-3, 12.7 g, 59.84 mmol, 1 eq) in DMF (150 mL) was added 1-(chloromethyl)-4-fluoro-1,4-diazoniabicyclo[2.2.2]octane; ditetrafluoroborate (Selectfluor™) (21.20 g, 59.84 mmol, 1 eq) and 2,6-dimethylpyridine (6.41 g, 59.84 mmol, 6.97 mL, 1 eq). The mixture was stirred at 25° C. for 10 hours after this time, LC-MS showed that the reaction was complete. The mixture was extracted with ethyl acetate (400 mL×3). The combined organic layers were dried over Na₂SO₄ and filtered. The filtrate was evaporated to dryness to give the crude product, which was purified by flash silica gel chromatography (ISCO®; 80 g SepaFlash® Silica Flash Column, Eluent of 22˜25% Ethyl acetate/Petroleum ether gradient @80 mL/min)., Petroleum ether/Ethyl acetate=3:1, Rf=0.4) to afford the product 4-(3,4-difluorophenyl)-5-fluorothiazol-2-amine, III-5 (8.9 g, 36.65 mmol, 61.2% yield, 94.8% purity) as a gray solid. LC-MS: (M+H)⁺=230.7; ¹H NMR (400 MHz, DMSO-d₆): 7.58 (br d, J=2.0 Hz, 1H), 7.55-7.51 (m, 1H), 7.45 (s, 1H), 7.06 (s, 2H)

### Preparation Example 8: Synthesis of 4-(4-chloro-3-fluorophenyl)-5-fluoro-1,3-thiazol-2-ylamine (III-6, Scheme 11)

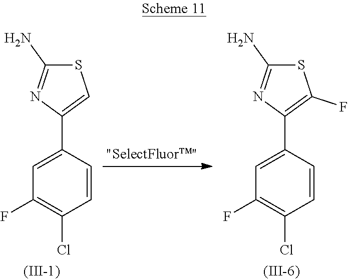 [SMILES: Nc1nc(-c2ccc(Cl)c(F)c2)cs1 ; Nc1nc(-c2ccc(Cl)c(F)c2)c(F)s1 ; "(III-1)" ; "(III-6)"]

To a solution of 4-(4-chloro-3-fluorophenyl)thiazol-2-amine (III-1, 1 g, 4.37 mmol, 1.0 eq) in MeCN (15 mL) was added 1-(chloromethyl) -4-fluoro-1,4-diazoniabicyclo[2.2.2]octane; ditetrafluoroborate (Selectfluor™) (1.55 g, 4.37 mmol, 1.0 eq) at 25° C. under N₂. The mixture was stirred at 25° C. for 16 hours. LC-MS showed ˜11% of the starting material remained. Several new peaks were shown on LC-MS and ˜21% of desired compound was detected. The reaction mixture was diluted with EtOAc (30 mL) and water (20 mL). The aqueous layer was extracted with EtOAc (2×20 mL). The combined organic phases were washed with water (2×15 mL) and brine (2×15 mL), dried with anhydrous Na₂SO₄, filtered and concentrated in vacuum. The residue was purified by silica gel chromatography (SiO₂, petroleum ether/EtOAc=0 to 100% EtOAc). The title compound (350 mg, 32% yield) was obtained as a brown solid.

### Preparation Example 9: Synthesis of N-(6-(N-(4-(4-chloro-3-fluorophenyl)thiazol-2-yl)sulfamoyl)pyridin-3-yl)acetamide (IV-1)

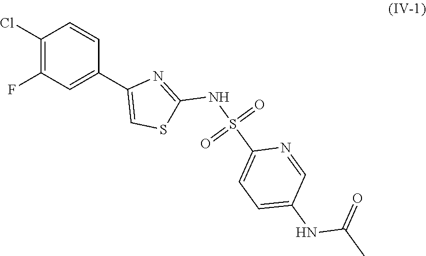 [SMILES: CC(=O)Nc1ccc(S(=O)(=O)Nc2nc(-c3ccc(Cl)c(F)c3)cs2)nc1]

To a solution of 4-(4-chloro-3-fluorophenyl)thiazol-2-amine (III-1) 1 g, 4.37 mmol, 1 eq) in pyridine (5 mL) was added 5-acetamidopyridine-2-sulfonyl chloride (II-1 2 g, 8.52 mmol, 1.95 eq) in one portion at 25° C. under N₂. The mixture was stirred at 25° C. for 4 hours. LC-MS showed compound II-1 was consumed completely and one main peak with desired m/z was detected. The mixture was poured into 1 M HCl (40 mL) and stirred for 10 min. The aqueous phase was filtered to afford the crude product The crude product was used without further purification.

### Preparation Example 10: Synthesis of N-(6-(N-(4-(3,4-difluorophenyl)-5-fluorothiazol-2-yl)sulfamoyl)-5-methylpyridin-3-yl)acetamide (IV-2)

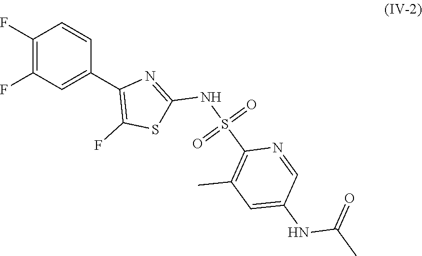 [SMILES: CC(=O)Nc1cnc(S(=O)(=O)Nc2nc(-c3ccc(F)c(F)c3)c(F)s2)c(C)c1]

A mixture of 5-acetamido-3-methylpyridine-2-sulfonyl chloride (II-2, 2.9 g, 11.66 mmol) and 4-(3,4-difluorophenyl)-5-fluorothiazol-2-amine (III-5, 2.68 g, 11.66 mmol) in pyridine (15 mL) was degassed and purged with N₂ for 3 times, and then the mixture was stirred at 25° C. for 16 hr under N₂ atmosphere. After this time, LC-MS showed that the reaction was complete. The reaction mixture was concentrated under reduced pressure to remove the solvent. The resulting residue was diluted with H₂O (50 mL) and extracted with ethyl acetate (30 mL×3). The combined organic layers were dried over Na₂SO₄, filtered and concentrated under reduced pressure to give a residue. The resulting crude product was purified by column chromatography (SiO₂, Petroleum ether/Ethyl acetate=100/0 to 20/80) to afford the compound IV-2 (3.47 g, 67.26% yield) as a brown solid. LC-MS: (M+H)⁺=443.0 Da.

### Preparation Example 11: Synthesis of 5-amino-N-(4-(4-chloro-3-fluorophenyl)thiazol-2-yl)pyridine-2-sulfonamide (V-1)

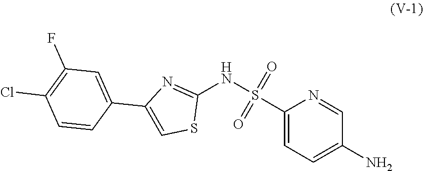 [SMILES: Nc1ccc(S(=O)(=O)Nc2nc(-c3ccc(Cl)c(F)c3)cs2)nc1]

To a solution of N-(6-(N-(4-(4-chloro-3-fluorophenyl)thiazol-2-yl)sulfamoyl)pyridin-3-yl)acetamide (IV-1, 1.5 g, 3.51 mmol, 1 eq) in H₂O (987 mg, 54.8 mmol, 988 μL, 15.6 eq) and EtOH (20 mL) was added NaOH (983 mg, 24.6 mmol, 7 eq) in one portion at 25° C. under N₂. The mixture was heated to 90° C. and stirred for 1.5 hours. LC-MS showed the starting material was consumed completely and one main peak with the desired mass was detected. The mixture was concentrated under reduced pressure. The residue was poured into water (10 mL) the pH of the mixture was adjusted to 7 with 1 M HCl, whereupon the residue was filtered to provide 5-amino-N-(4-(4-chloro-3-fluorophenyl)thiazol-2-yl)pyridine-2-sulfonamide (V-1) (1 g, 74% yield, yellow solid) which was used without further purification.

### Preparation Example 12: Synthesis of 5-amino-N-(4-(3,4-difluorophenyl)-5-fluorothiazol-2-yl)-3-methylpyridine-2-sulfonamide (V-2)

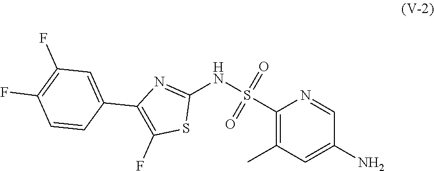 [SMILES: Cc1cc(N)cnc1S(=O)(=O)Nc1nc(-c2ccc(F)c(F)c2)c(F)s1]

To a solution of N-(6-(N-(4-(3,4-difluorophenyl)-5-fluorothiazol-2-yl)sulfamoyl)-5-methylpyridin-3-yl)acetamide (IV-2, 4.64 g, 10.49 mmol) in ethanol (50 mL) and water (50 mL) was added sodium hydroxide (3.36 g, 83.90 mmol). The mixture was stirred at 80° C. for 0.5 hour. After this time, LC-MS showed that the reaction was complete. The reaction mixture was extracted with ethyl acetate (500 mL×3), washed with brine (120 mL×2), dried over Na₂SO₄ and concentrated to afford the crude 5-amino-N-(4-(3,4-difluorophenyl)-5-fluorothiazol-2-yl)-3-methylpyridine-2-sulfonamide (V-2, 3.77 g, 90% yield) as a red oil. LC-MS: (M+H)⁺=400.9; ¹H NMR (400 MHz, DMSO-d₆): 7.64-7.54 (m, 2H), 7.54-7.47 (m, 1H), 7.47-7.37 (m, 1H), 6.72 (d, J=2.0 Hz, 1H), 5.60 (br s, 2H), 2.42 (s, 3H).

### Preparation Example 13: Synthesis of N-(4-(4-chloro-3-fluorophenyl)thiazol-2-yl)-5-((2-hydroxy-3-methoxybenzylidene)amino)pyridine-2-sulfonamide (VII-1)

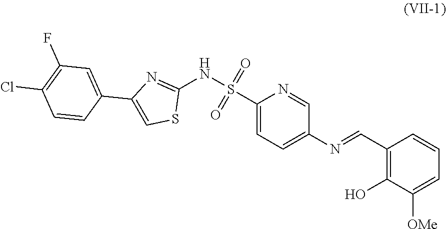 [SMILES: COc1cccc(/C=N/c2ccc(S(=O)(=O)Nc3nc(-c4ccc(Cl)c(F)c4)cs3)nc2)c1O]

To a mixture of 5-amino-N-(4-(4-chloro-3-fluorophenyl)thiazol-2-yl)pyridine-2-sulfonamide (V-1, 200 mg, 520 μmol, 1 eq) and 2-hydroxy-3-methoxybenzaldehyde (118.6 mg, 780 μmol, 1.5 eq.; Formula VI,) in EtOH (5 mL) was added trifluoroacetic acid (59.3 mg, 519.7 μmol, 38.5 μL, 1 eq.) in one portion at 25° C. under N₂. The mixture was heated to 25° C. and stirred for 7 hours. LC-MS indicated the starting amine was consumed completely. The mixture was cooled to 25° C. and concentrated in reduced pressure. The crude product of N-(4-(4-chloro-3-fluorophenyl)thiazol-2-yl)-5-((2-hydroxy-3-methoxybenzylidene)amino)pyridine-2-sulfonamide (VII-1, 260 mg, 96% yield) was used into the next step without further purification.

### Preparation Example 14: Synthesis of N-(4-(3,4-difluorophenyl)-5-fluorothiazol-2-yl)-5-((2-hydroxy-3-methoxybenzylidene)amino)-3-methylpyridine-2-sulfonamide (VII-2)

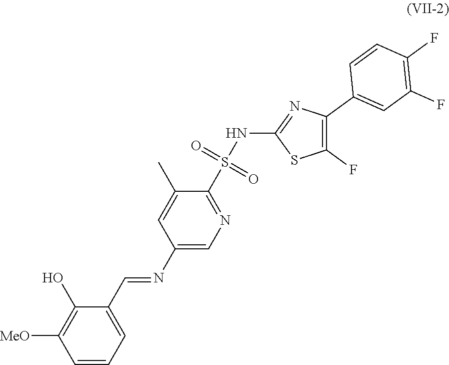 [SMILES: COc1cccc(/C=N/c2cnc(S(=O)(=O)Nc3nc(-c4ccc(F)c(F)c4)c(F)s3)c(C)c2)c1O]

To a solution of 2-hydroxy-3-methoxy-benzaldehyde (567 mg, 3.75 mmol) in toluene (10 mL) was added 5-amino-N-(4-(3,4-difluorophenyl)-5-fluorothiazol-2-yl)-3-methylpyridine-2-sulfonamide (V-2, 1 g, 2.50 mmol). The mixture was stirred at 140° C. for 2 hours. After this time, LC-MS showed that the reaction was complete. The solvent was removed under pressure reduction to afford the crude N-(4-(3,4-difluorophenyl)-5-fluorothiazol-2-yl)-5-((2-hydroxy-3-methoxybenzylidene)amino)-3-methylpyridine-2-sulfonamide (VII-2, 1.2 g, crude) as a brown solid.

### Preparation of Compounds of Formula (I)

### Preparation Example 15: Synthesis of N-(4-(4-chloro-3-fluorophenyl)thiazol-2-yl)-5-((2-hydroxy-3-methoxybenzyl)amino)pyridine-2-sulfonamide (compound I-8)

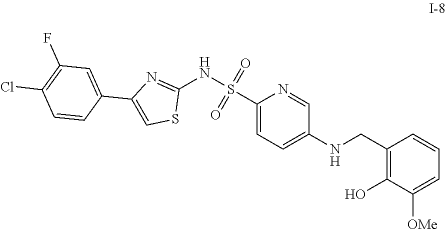 [SMILES: COc1cccc(CNc2ccc(S(=O)(=O)Nc3nc(-c4ccc(Cl)c(F)c4)cs3)nc2)c1O]

To a solution of N-(4-(4-chloro-3-fluorophenyl)thiazol-2-yl)-5-((2-hydroxy-3-methoxybenzylidene)amino)pyridine-2-sulfonamide (VII-1, 260 mg, 501 μmol, 1 eq) in DMF (5 mL) was added sodium triacetoxyborohydride (STAB, 425 mg, 2.00 mmol, 4 eq) in one portion at 25° C. under N₂. The mixture was stirred at 25° C. for 16 hours. LC-MS showed the starting material was consumed completely and one main peak with desired m/z was detected. The reaction mixture was poured into water (50 mL) and stirred for 10 min. The aqueous phase was filtered to get crude product. The residue was purified by preparatory high pressure liquid chromatography (TFA condition) to provide N-(4-(4-chloro-3-fluorophenyl)thiazol-2-yl)-5-((2-hydroxy-3-methoxybenzyl)amino)pyridine-2-sulfonamide, I-8 as a white solid (58.6 mg, 22% yield).

### Preparation Example 16: Synthesis of N-(4-(3,4-difluorophenyl)-5-fluorothiazol-2-yl)-5-((2-hydroxy-3-methoxybenzyl)amino)-3-methylpyridine-2-sulfonamide (I-18)

Compound I-18 may be prepared according to Scheme 12:

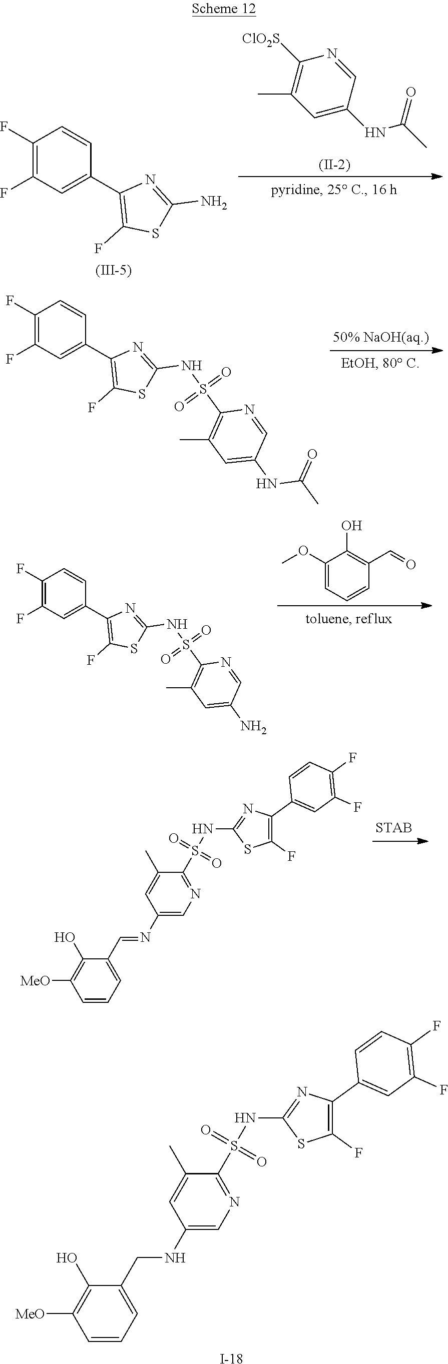

To a solution of N-(4-(3,4-difluorophenyl)-5-fluorothiazol-2-yl)-5-((2-hydroxy-3-methoxybenzylidene)amino)-3-methylpyridine-2-sulfonamide (VII-2, 1.2 g, 2.24 mmol) in DCE (15 mL) was added sodium triacetoxyborohydride (9.52 g, 44.90 mmol) at 25° C. The mixture was stirred at 25° C. for 0.5 hr. After this time, LC-MS showed that the reaction was complete. The reaction mixture was diluted with ethyl acetate (30 mL×3), washed with brine (50 mL×2), dried over Na₂SO₄ and concentrated to afford residue. The residue was purified by column chromatography (silica gel, 100-200 mesh, 60% ethyl acetate in petroleum ether) to afford N-(4-(3,4-difluorophenyl)-5-fluorothiazol-2-yl)-5-((2-hydroxy-3-methoxybenzyl)amino)-3-methylpyridine-2-sulfonamide (I-18, 0.360 g, 29.9% yield) as a brown solid. LC-MS:(M+H)⁺=537.0; ¹H NMR (400 MHz, DMSO-d₆): 8.82 (s, 1H), 7.76 (d, J=2.3 Hz, 1H), 7.70-7.63 (m, 1H), 7.61-7.54 (m, 1H), 7.53 (br d, J=3.4 Hz, 1H), 7.02 (br t, J=5.4 Hz, 1H), 6.89-6.83 (m, 2H), 6.79-6.69 (m, 2H), 4.27 (br d, J=5.8 Hz, 2H), 3.79 (s, 3H), 2.46 (s, 3H)

DCE/MeOH and DCE/DMF also can be used instead of DCE, for example, to improve reagent solubility.

In a manner and with changes to the processes and methods described herein that are evident to a person of ordinary skill in the art, the compounds in Table 1 were prepared.

TABLE 1

| Cmpd. | | |
|---|---|---|
| No. | Structure | NMR data |
| I-1 | 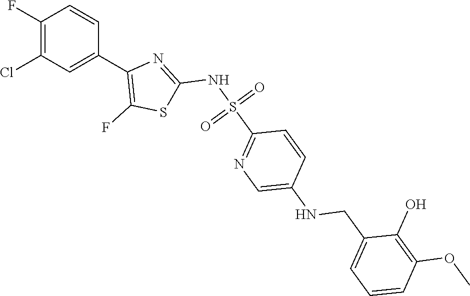 [SMILES: COc1cccc(CNc2ccc(S(=O)(=O)Nc3nc(-c4ccc(F)c(Cl)c4)c(F)s3)nc2)c1O] | ¹H NMR (400 MHz, DMSO-d6) δ 8.86 (s, 1H), 7.99 (d, J = 2.7 Hz, 1H), 7.81 (dd, J = 7.1, 2.1 Hz, 1H), 7.70-7.61 (m, 2H), 7.55 (t, J = 9.0 Hz, 1H), 7.25 (s, 1H), 6.95 (dd, J= 8.8, 2.7 Hz, 1H), 6.86 (dd, J = 7.8, 1.5 Hz, 1H), 6.76 (dd, J = 7.6, 1.4 Hz, 1H), 6.71 (t, J = 7.8 Hz, 1H), 4.28 (s, 2H), 3.79 (s, 3H). |
| I-2 | 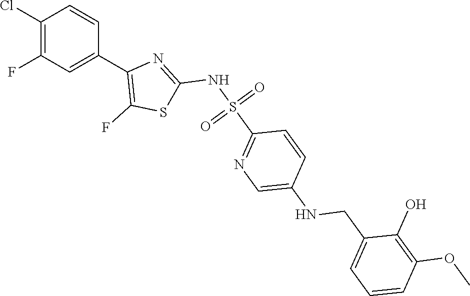 [SMILES: COc1cccc(CNc2ccc(S(=O)(=O)Nc3nc(-c4ccc(Cl)c(F)c4)c(F)s3)nc2)c1O] | ¹H NMR (400 MHz, DMSO-d6) δ 8.82 (s, 1H), 7.98 (d, J = 2.6 Hz, 1H), 7.69 (dd, J= 17.8, 8.6 Hz, 2H), 7.61 (dd, J = 10.7, 1.9 Hz, 1H), 7.50 (dd, J = 8.4, 1.6 Hz, 1H), 7.19 (t, J = 5.6 Hz, 1H), 6.94 (dd, J = 8.8, 2.7 Hz, 1H), 6.85 (dd, J = 7.9, 1.5 Hz, 1H), 6.76 (dd, J = 7.6, 1.1 Hz, 1H), 6.70 (t, J = 7.8 Hz, 1H), 4.27 (d, J = 5.6 Hz, 2H), 3.79 (s, 3H). |
| I-3 | 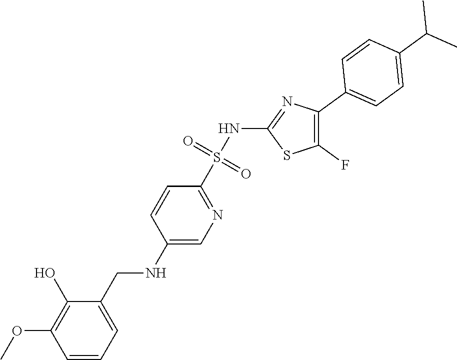 [SMILES: COc1cccc(CNc2ccc(S(=O)(=O)Nc3nc(-c4ccc(C(C)C)cc4)c(F)s3)nc2)c1O] | ¹H NMR (400 MHz, DMSO-d6) δ 8.84 (s, 1H), 7.98 (d, J = 2.6 Hz, 1H), 7.65 (d, J = 8.7 Hz, 1H), 7.54 (d, J = 8.2 Hz, 2H), 7.36 (d, J = 8.3 Hz, 2H), 6.94 (dd, J = 8.8, 2.7 Hz, 1H), 6.86 (dd, J = 7.8, 1.2 Hz, 1H), 6.80-6.75 (m, 1H), 6.71 (t, J = 7.8 Hz, 1H), 4.28 (s, 2H), 3.79 (s, 3H), 2.95-2.88 (m, 1H), 1.21 (d, J = 6.9 Hz, 6H). |
| I-4 | 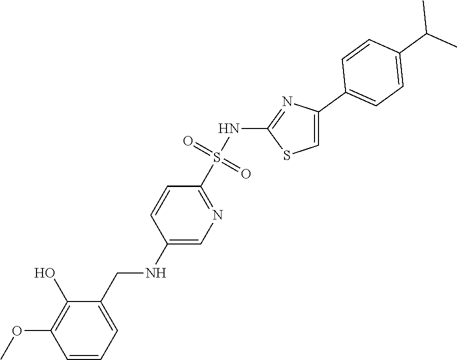 [SMILES: COc1cccc(CNc2ccc(S(=O)(=O)Nc3nc(-c4ccc(C(C)C)cc4)cs3)nc2)c1O] | ¹H NMR (400 MHz, DMSO-d6) δ 13.01 (s, 1H), 8.83 (s, 1H), 7.95 (d, J = 2.4 Hz, 1H), 7.67-7.58 (m, 3H), 7.30 (d, J = 8.2 Hz, 2H), 7.06 (s, 2H), 6.91 (dd, J = 8.6, 2.5 Hz, 1H), 6.86 (dd, J = 7.8, 0.9 Hz, 1H), 6.77 (d, J = 6.9 Hz, 1H), 6.71 (t, J = 7.7 Hz, 1H), 4.26 (d, J = 5.8 Hz, 2H), 3.79 (s, 3H), 2.90 (dt, J = 13.6, 6.8 Hz, 1H), 1.20 (d, J = 6.9 Hz, 6H). |
| I-5 | 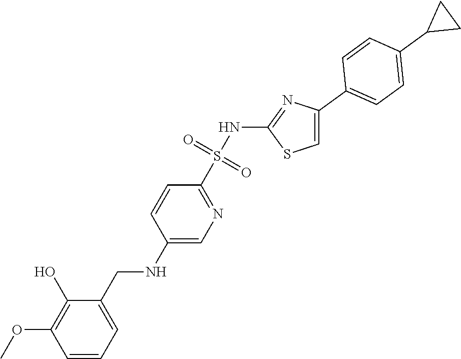 [SMILES: COc1cccc(CNc2ccc(S(=O)(=O)Nc3nc(-c4ccc(C5CC5)cc4)cs3)nc2)c1O] | ¹H NMR (400 MHz, DMSO-d6) δ 13.01 (s, 1H), 8.83 (s, 1H), 7.94 (d, J = 2.4 Hz, 1H), 7.63 (d, J = 8.7 Hz, 1H), 7.57 (d, J = 8.2 Hz, 2H), 7.12 (d, J = 8.2 Hz, 2H), 7.07 (s, 1H), 6.91 (dd, J = 8.7, 2.5 Hz, 1H), 6.85 (d, J = 7.8 Hz, 1H), 6.76 (d, J = 6.8 Hz, 1H), 6.70 (t, J = 7.7 Hz, 1H), 4.26 (s, 2H), 3.78 (s, 3H), 1.96-1.90 (m, 1H), 1.00- 0.93 (m, 2H), 0.74-0.67 (m, 2H). |
| I-6 | 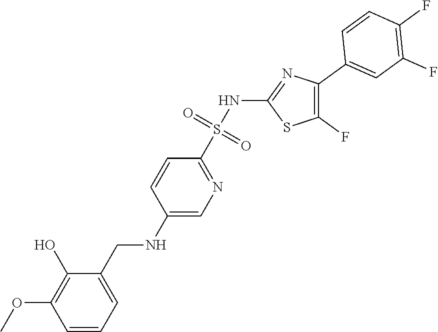 [SMILES: COc1cccc(CNc2ccc(S(=O)(=O)Nc3nc(-c4ccc(F)c(F)c4)c(F)s3)nc2)c1O] | ¹H NMR (400 MHz, DMSO-d6) δ 8.83 (s, 1H), 7.99 (d, J = 2.7 Hz, 1H), 7.70- 7.60 (m, 2H), 7.60-7.53 (m, 1H), 7.52-7.47 (m, 1H), 7.23 (s, 1H), 6.95 (dd, J = 8.8, 2.7 Hz, 1H), 6.86 (dd, J = 7.9, 1.4 Hz, 1H), 6.77 (dd, J = 7.7, 1.5 Hz, 1H), 6.71 (t, J = 7.8 Hz, 1H), 4.28 (s, 2H), 3.79 (s, 3H). |
| I-7 | 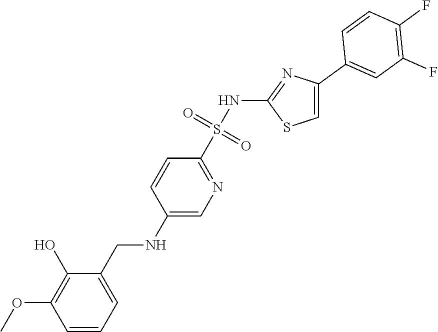 [SMILES: COc1cccc(CNc2ccc(S(=O)(=O)Nc3nc(-c4ccc(F)c(F)c4)cs3)nc2)c1O] | ¹H NMR (400 MHz, DMSO-d6) δ ppm 3.78 (s, 3 H) 4.26 (s, 2 H) 6.67-6.80 (m, 2 H) 6.85 (dd, J = 7.83, 1.10 Hz, 1 H) 6.92 (dd, J = 8.68, 2.69 Hz, 1 H) 7.31 (br s, 1 H) 7.47-7.73 (m, 3 H) 7.84 (br t, J = 8.93 Hz, 1 H) 7.90-8.00 (m, 1 H) 8.85 (br s, 1 H) 13.09 (br s, 1 H) |
| I-8 | 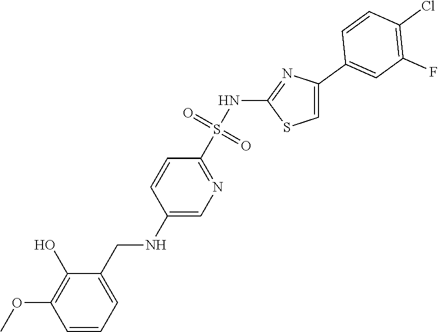 [SMILES: COc1cccc(CNc2ccc(S(=O)(=O)Nc3nc(-c4ccc(Cl)c(F)c4)cs3)nc2)c1O] | ¹H NMR (400 MHz, DMSO-d6) δ 3.78 (s, 3H) 4.26 (d, J = 5.87 Hz, 2H) 6.67- 6.73 (m, 1H) 6.75 (d, J = 7.32 Hz, 1H) 6.85 (d, J = 7.52 Hz, 1H) 6.91 (d, J = 8.03 Hz, 1H) 7.10 (br s, 1H) 7.38 (br s, 1H) 7.57-7.70 (m, 3H) 7.80 (dd, J = 10.82, 1.77 Hz, 1H) 7.94 (d, J = 2.69 Hz, 1H) 8.83 (s, 1H) 13.05 (br s, 1H) |
| I-9 | 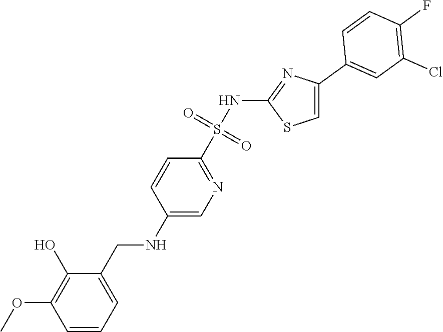 [SMILES: COc1cccc(CNc2ccc(S(=O)(=O)Nc3nc(-c4ccc(F)c(Cl)c4)cs3)nc2)c1O] | ¹H NMR (400 MHz, DMSO-d6) δ 13.06 (s, 1H), 8.82 (s, 1H), 8.02- 7.94 (m, 2H), 7.75 (ddd, J = 8.4, 4.4, 2.2 Hz, 1H), 7.66 (d, J = 8.6 Hz, 1H), 7.51 (t, J = 9.0 Hz, 1H), 7.32 (s, 1H), 7.11 (s, 1H), 6.93 (dd, J = 8.7, 2.4 Hz, 1H), 6.86 (d, J = 7.8 Hz, 1H), 6.77 (d, J = 7.1 Hz, 1H), 6.71 (t, J = 7.8 Hz, 1H), 4.27 (d, J = 5.7 Hz, 2H), 3.79 (s, 3H). |
| I-10 | 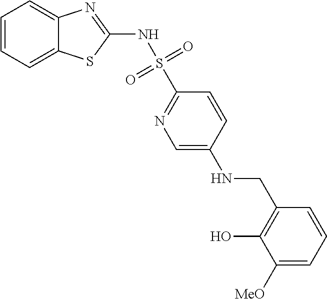 [SMILES: COc1cccc(CNc2ccc(S(=O)(=O)Nc3nc4ccccc4s3)nc2)c1O] | ¹HNMR (400 MHz, DMSO-d6) δ 8.77 (s, 1H), 8.18 (s, 1H), 7.90 (d, J = 2.6 Hz, 1H), 7.60 (d, J = 8.6 Hz, 2H), 7.30- 7.17 (m, 2H), 7.09-7.01 (m, 1H), 6.90-6.82 (m, 2H), 6.76 (dd, J = 7.7, 1.4 Hz, 1H), 6.70 (t, J = 7.8 Hz, 1H), 4.24 (d, J= 6.1 Hz, 2H), 3.79 (s, 3H). |
| I-11 | 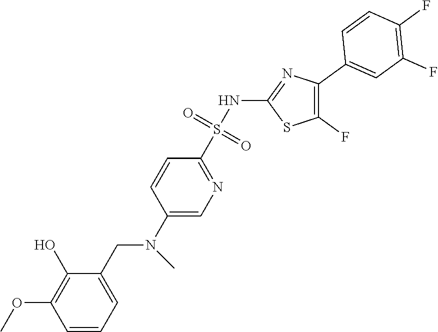 [SMILES: COc1cccc(CN(C)c2ccc(S(=O)(=O)Nc3nc(-c4ccc(F)c(F)c4)c(F)s3)nc2)c1O] | ¹H NMR (400 MHz, DMSO-d6) δ 12.76 (s, 1H), 8.90 (s, 1H), 8.04 (d, J = 2.6 Hz, 1H), 7.74-7.60 (m, 2H), 7.52 (ddd, J = 13.0, 10.7, 5.6 Hz, 2H), 7.07 (dd, J = 9.0, 2.9 Hz, 1H), 6.85 (d, J = 8.0 Hz, 1H), 6.67 (t, J = 7.9 Hz, 1H), 6.50 (d, J = 7.6 Hz, 1H), 4.58 (s, 2H), 3.78 (s, 3H), 3.12 (s, 3H) |
| I-12 | 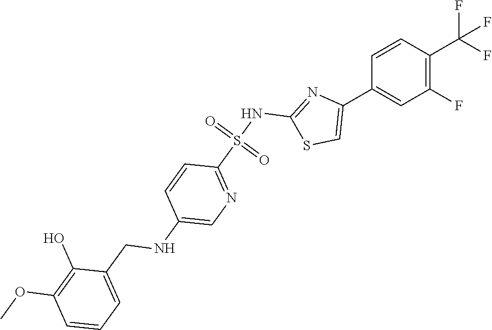 [SMILES: COc1cccc(CNc2ccc(S(=O)(=O)Nc3nc(-c4ccc(C(F)(F)F)c(F)c4)cs3)nc2)c1O] | ¹H NMR (400 MHz, DMSO-d6) δ 8.85 (s, 1H), 7.97 (s, 1H), 7.87 (dd, J = 21.1, 10.0 Hz, 2H), 7.77 (d, J = 8.3 Hz, 1H), 7.74-7.55 (m, 2H), 6.94 (d, J = 8.7 Hz, 1H), 6.88-6.83 (m, 1H), 6.76 (d, J = 7.4 Hz, 1H), 6.70 (t, J = 7.8 Hz, 1H), 4.27 (s, 2H), 3.79 (s, 3H). |
| I-13 | 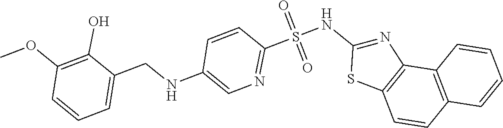 [SMILES: COc1cccc(CNc2ccc(S(=O)(=O)Nc3nc4c(ccc5ccccc54)s3)nc2)c1O] | ¹H NMR (400 MHz, DMSO-d6) δ 13.84 (s, 1H), 8.83 (s, 1H), 8.37 (d, J = 8.2 Hz, 1H), 8.03 (d, J = 7.8 Hz, 1H), 7.96 (d, J = 2.7 Hz, 1H), 7.88 (d, J = 8.7 Hz, 1H), 7.81 (d, J = 8.7 Hz, 1H), 7.70 (d, J = 8.7 Hz, 1H), 7.67- 7.56 (m, 2H), 6.94 (dd, J = 8.8, 2.7 Hz, 1H), 6.85 (dd, J = 7.8, 1.5 Hz, |
| | | 1H), 6.75 (dd, J = 7.6, 1.4 Hz, 1H), |
| | | 6.70 (t, J = 7.8 Hz, 1H), 4.26 (s, 2H), |
| | | 3.78 (s, 3H). |
| I-14 | 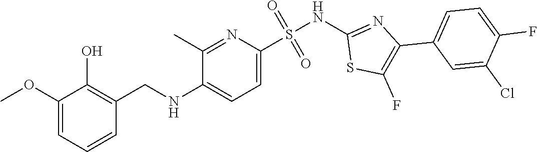 [SMILES: COc1cccc(CNc2ccc(S(=O)(=O)Nc3nc(-c4ccc(F)c(Cl)c4)c(F)s3)nc2C)c1O] | ¹H NMR (400 MHz, DMSO-d6) δ 8.86 (s, 1H), 7.80 (dd, J = 7.0, 2.0 Hz, 1H), 7.64 (ddd, J = 8.2, 4.5, 2.0 Hz, 1H), 7.55 (dd, J = 17.3, 8.6 Hz, 2H), 7.25- 6.95 (m, 2H), 6.83 (dd, J = 6.7, 2.7 Hz, 1H), 6.67 (dd, J = 7.3, 4.6 Hz, 3H), 4.35 (d, J = 3.9 Hz, 2H), 3.79 (s, 3H), 2.38 (s, 3H) |
| I-15 | 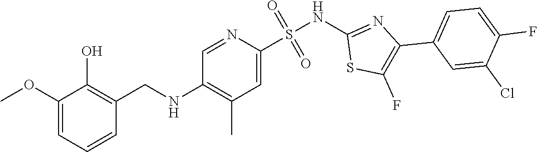 [SMILES: COc1cccc(CNc2cnc(S(=O)(=O)Nc3nc(-c4ccc(F)c(Cl)c4)c(F)s3)cc2C)c1O] | ¹H NMR (400 MHz, DMSO-d6) δ 8.89 (s, 1H), 7.79 (dd, J = 7.1, 2.2 Hz, 1H), 7.67-7.59 (m, 3H), 7.55 (t, J = 9.0 Hz, 1H), 6.82 (dd, J = 7.8, 1.5 Hz, 1H), 6.72 (dd, J = 7.6, 1.4 Hz, 1H), 6.67 (t, J = 7.7 Hz, 1H), 6.60 (t, J = 6.0 Hz, 1H), 4.42 (d, J = 5.2 Hz, 2H), 3.78 (s, 3H0, 2.23 (s, 3H) |
| I-16 | 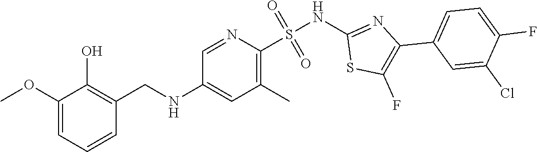 [SMILES: COc1cccc(CNc2cnc(S(=O)(=O)Nc3nc(-c4ccc(F)c(Cl)c4)c(F)s3)c(C)c2)c1O] | ¹H NMR (400 MHz, DMSO-d6) δ 8.84 (s, 1H), 7.82 (d, J = 6.8Hz, 1H), 7.66 (m, 1H), 7.55 (t, J = 8.9 Hz, 1H), 6.86 (d, J = 9.2Hz, 2H), 6.77 (d, J = 7.6 Hz, 1H), 6.70 (t, J = 7.7 Hz), 4.26 (s, 2H), 3.79 (s, 3H), 2.45 (s, 3H) |
| I-17 | 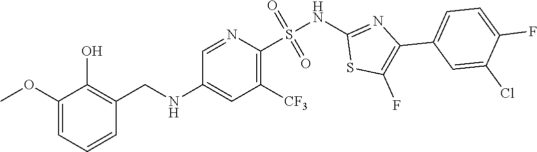 [SMILES: COc1cccc(CNc2cnc(S(=O)(=O)Nc3nc(-c4ccc(F)c(Cl)c4)c(F)s3)c(C(F)(F)F)c2)c1O] | ¹H NMR (400 MHz, DMSO-d6) δ 8.91 (s, 1H), 8.04 (s, 1H), 7.83 (d, J = 6.4 Hz, 1H), 7.66 (dd, J = 7.2, 2.4 Hz, 1H), 7.54 (m, 2H), 7.34 (s, 1H), 6.87 (d, J = 7.5 Hz, 1H), 6.78 (d, J = 7.3 Hz, 1H), 4.33 (d, J = 5.1 Hz, 2H), 3.78 (s, 3H) |
| I-18 | 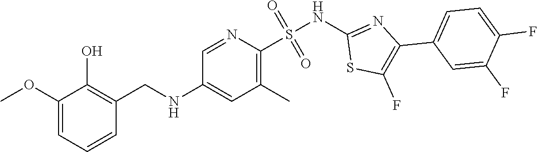 [SMILES: COc1cccc(CNc2cnc(S(=O)(=O)Nc3nc(-c4ccc(F)c(F)c4)c(F)s3)c(C)c2)c1O] | ¹H NMR (400 MHz, DMSO-d6): δ 8.82 (s, 1H), 7.76 (d, J = 2.3 Hz, 1H), 7.70- 7.63 (m, 1H), 7.61-7.54 (m, 1H), 7.53 (br d, J = 3.4 Hz, 1H), 7.02 (br t, J = 5.4 Hz, 1H), 6.89-6.83 (m, 2H), 6.79-6.69 (m, 2H), 4.27 (br d, J = 5.8 Hz, 2H), 3.79 (s, 3H), 2.46 (s, 3H) |
| I-19 | 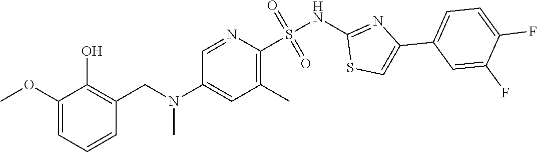 [SMILES: COc1cccc(CN(C)c2cnc(S(=O)(=O)Nc3nc(-c4ccc(F)c(F)c4)cs3)c(C)c2)c1O] | ¹H NMR (400 MHz, DMSO-d6) δ 13.14 (s, 1H), 8.92 (s, 1H), 7.84 (dd, J = 24.5, 14.1 Hz, 2H), 7.55 (m, 2H), 7.29 (s, 1H), 6.99 (d, J = 16.9 Hz, 1H), 6.85 (d, J = 7.0 Hz, 1H), 6.67 (t, J = 7.1 Hz, 1H), 6.50 (d, J = 6.8 Hz, 1H, 4.57 (s, 2H), 3.79 (s, 3H), 3.08 (s, 3H), 2.52 (s, 3H) |
| I-20 | 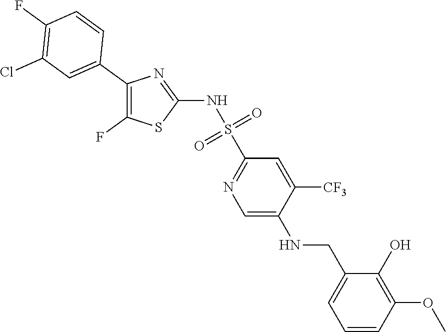 [SMILES: COc1cccc(CNc2cnc(S(=O)(=O)Nc3nc(-c4ccc(F)c(Cl)c4)c(F)s3)cc2C(F)(F)F)c1O] | ¹H NMR (400 MHz, DMSO-d6) δ 8.99 (m, 1H), 8.03 (m, 2H), 7.77 (m, 2H), 7.62 (m, 1H), 7.52 (m, 1H), 7.18 (m, 1H), 6.83 (m, 1H), 6.71 (m, 2H), 4.52 (m, 2H), 3.78 (s, 3H) |
| I-21 | 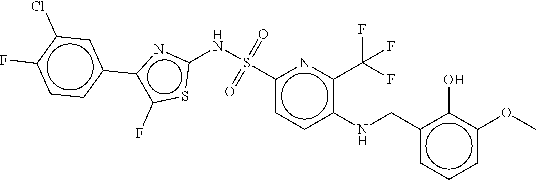 | TBD |
| I-22 | 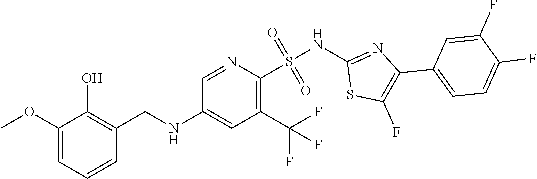 [SMILES: COc1cccc(CNc2cnc(S(=O)(=O)Nc3nc(-c4ccc(F)c(F)c4)c(F)s3)c(C(F)(F)F)c2)c1O] | ¹H NMR (400 MHz, DMSO-d6): 8.88 (s, 1H), 8.03 (d, J = 1.9 Hz, 1H), 7.71- 7.63 (m, 1H), 7.60-7.51 (m, 2H), 7.47 (br s, 1H), 7.33 (br s, 1H), 6.87 (dd, J = 1.4, 8.0 Hz, 1H), 6.81-6.76 (m, 1H), 6.75-6.69 (m, 1H), 4.34 (d, J = 5.8 Hz, 2H), 3.79 (s, 3H) |
| I-23 | 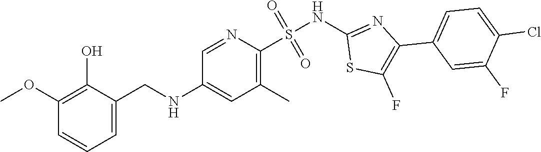 [SMILES: COc1cccc(CNc2cnc(S(=O)(=O)Nc3nc(-c4ccc(Cl)c(F)c4)c(F)s3)c(C)c2)c1O] | ¹H NMR (400 MHz, DMSO-d6): 8.82 (s, 1H), 7.75 (d, J = 2.4 Hz, 1H), 7.70 (t, J = 8.2 Hz, 1H), 7.62 (dd, J = 1.9, 10.6 Hz, 1H), 7.51 (dd, J = 1.6, 8.4 Hz, 1H), 7.05-7.00 (m, 1H), 6.87- 6.82 (m, 2H), 6.78-6.75 (m, 1H), 6.73-6.67 (m, 1H), 4.26 (br d, J = 5.8 Hz, 2H), 3.78 (s, 3H), 2.45 (s, 3H) |
| I-24 | 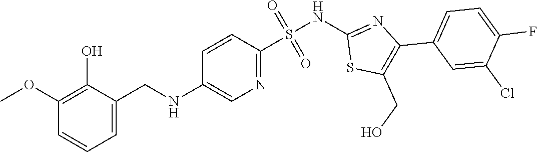 [SMILES: COc1cccc(CNc2ccc(S(=O)(=O)Nc3nc(-c4ccc(F)c(Cl)c4)c(CO)s3)nc2)c1O] | ¹H NMR (400 MHz, DMSO-d6): 8.86- 8.74 (m, 1H), 7.93 (d, J = 2.5 Hz, 1H), 7.76 (d, J = 6.4 Hz, 1H), 7.61 (br d, J = 8.6 Hz, 1H), 7.55-7.48 (m, 2H), 7.01-6.94 (m, 1H), 6.93-6.83 (m, 2H), 6.79-6.75 (m, 1H), 6.74-6.67 (m, 1H), 4.40 (br d, J = 4.6 Hz, 2H), 4.26 (br d, J = 5.9 Hz, 2H), 3.79 (s, 3H) |
| I-25 | 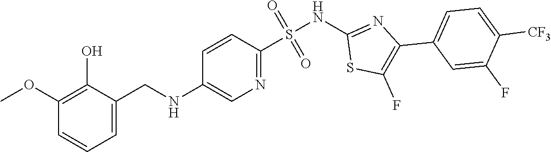 [SMILES: COc1cccc(CNc2ccc(S(=O)(=O)Nc3nc(-c4ccc(C(F)(F)F)c(F)c4)c(F)s3)nc2)c1O] | 1H NMR (400 MHz, DMSO-d6): 8.80- 8.75 (m, 1H), 7.90 (br d, J = 8.4 Hz, 1H), 7.79 (br t, J = 7.6 Hz, 1H), 7.63- 7.53 (m, 3H), 7.03 (br s, 1H), 6.84 (br d, J = 8.6 Hz, 1H), 6.78 (br d, J = 7.6 Hz, 1H), 6.71-6.66 (m, 1H), 6.65-6.59 (m, 1H), 4.19 (br d, J = 4.9 Hz, 2H), 3.72 (s, 3H) |
| I-26 | 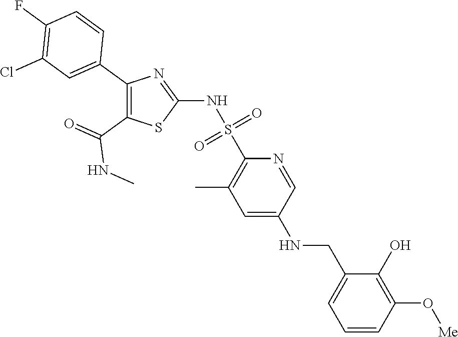 [SMILES: CNC(=O)c1sc(NS(=O)(=O)c2ncc(NCc3cccc(OC)c3O)cc2C)nc1-c1ccc(F)c(Cl)c1] | TBD |
| I-27 | 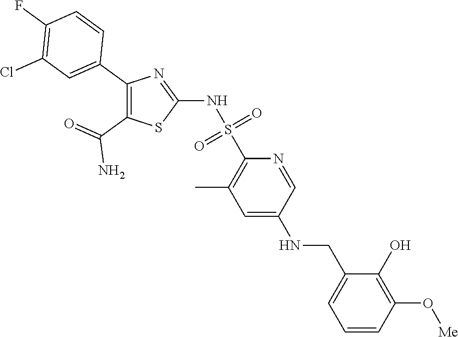 [SMILES: COc1cccc(CNc2cnc(S(=O)(=O)Nc3nc(-c4ccc(F)c(Cl)c4)c(C(N)=O)s3)c(C)c2)c1O] | TBD |
| I-28 | 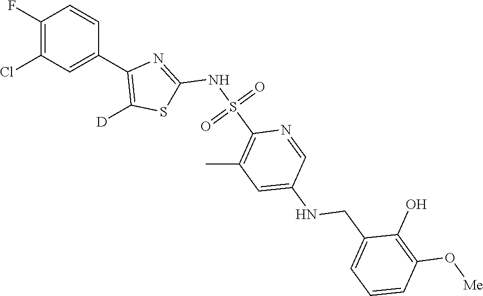 [SMILES: [2H]c1sc(NS(=O)(=O)c2ncc(NCc3cccc(OC)c3O)cc2C)nc1-c1ccc(F)c(Cl)c1] | TBD |
| I-29 | 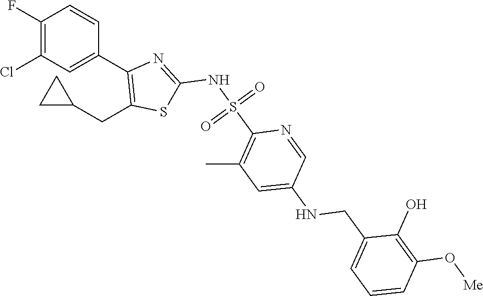 [SMILES: COc1cccc(CNc2cnc(S(=O)(=O)Nc3nc(-c4ccc(F)c(Cl)c4)c(CC4CC4)s3)c(C)c2)c1O] | 1H NMR (400 MHz, DMSO-d6) δ = 13.04-12.18 (m, 1H), 8.80 (s, 1H), 7.84 -7.67 (m, 2H), 7.61-7.42 (m, 2H), 6.85 (br d, J = 7.3 Hz, 2H), 6.82- 6.75 (m,2H), 6.75-6.67 (m, 1H), 4.26 (br d, J = 5.8 Hz, 2H), 3.79 (s, 3H), 2.44 (s, 3H), 1.05-0.78 (m, 1H), 0.61-0.37 (m, 2H), 0.29-0.04 (m, 2H); 19F NMR (376 MHz, DMSO-d6) δ = −115.47 (brs, 1F) |
| I-30 | 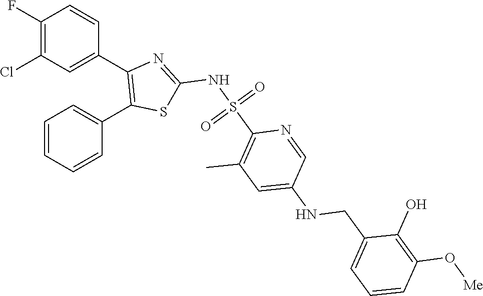 [SMILES: COc1cccc(CNc2cnc(S(=O)(=O)Nc3nc(-c4ccc(F)c(Cl)c4)c(-c4ccccc4)s3)c(C)c2)c1O] | ¹H NMR (400 MHz, DMSO-d6) δ = 8.80 (s, 1H), 7.74 (d, J = 2.0 Hz, 1H), 7.62 (br d, J = 6.5 Hz, 1H), 7.44- 7.37 (m, 1H), 7.37 - 7.26 (m, 4H), 7.25-7.17 (m,2H), 6.90 (br s, 1H), 6.85 (br d, J = 7.8 Hz, 1H), 6.83- 6.75 (m, 2H), 6.74-6.65 (m, 1H), 4.26 (br d, J = 5.9 Hz, 2H), 3.79 (s, 3H), 2.46 (s, 3H); ¹⁹F NMR (376 MHz, DMSO-d6) δ = −114.41- −115.30 (m, 1F) |
| I-31 | 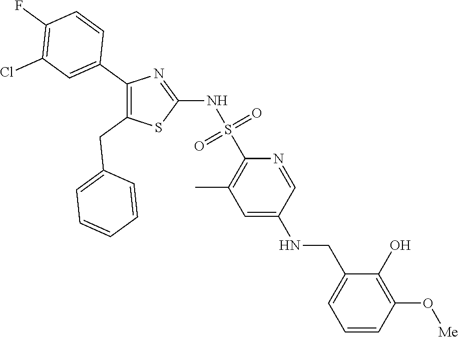 [SMILES: COc1cccc(CNc2cnc(S(=O)(=O)Nc3nc(-c4ccc(F)c(Cl)c4)c(Cc4ccccc4)s3)c(C)c2)c1O] | ¹H NMR (400 MHz, DMSO-d6) δ = 12.87-12.52 (m, 1H), 8.81 (s, 1H), 7.80-7.71 (m, 1H), 7.68 (d, J = 2.3 Hz, 1H), 7.53 (d, J = 7.4 Hz, 2H), 7.36-7.28 (m,2H), 7.27-7.21 (m, 1H), 7.17 (d, J = 7.0 Hz, 2H), 6.90- 6.82 (m, 2H), 6.80-6.75 (m, 2H), 6.74-6.66 (m, 1H), 4.25 (d, J = 5.9 Hz, 2H), 3.96 (s, 2H), 3.79 (s,3H), 2.41 (s, 3H); ¹⁹F NMR (377 MHz, DMSO-d6) δ = −114.41-−115.52 (m, 1F) |
| I-32 | 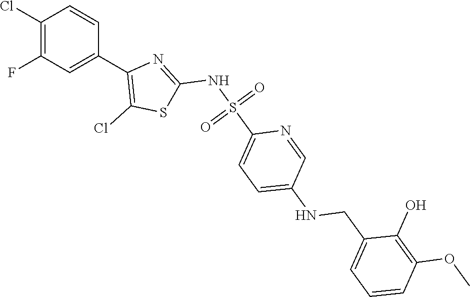 [SMILES: COc1cccc(CNc2ccc(S(=O)(=O)Nc3nc(-c4ccc(Cl)c(F)c4)c(Cl)s3)nc2)c1O] | ¹H NMR (400 MHZ, DMSO-d6) δ = 8.83 (s, 1H), 7.98 (d, J = 2.6 Hz, 1H), 7.76-7.69 (m, 2H), 7.66 (d, J = 8.8 Hz, 1H), 7.56 (br d, J = 7.6 Hz, 1H), 7.25-7.17 (m, 1H), 6.99- 6.90 (m, 1H), 6.86 (d, J = 7.5 Hz, 1H), 6.79-6.74 (m, 1H), 6.74- 6.68 (m, 1H), 4.27 (br d, J = 5.3 Hz, 2H), 3.79 (s, 3H); 19F NMR (377 MHz, DMSO-d6) δ = −115.08 (br s, 1F) |
| I-33 | 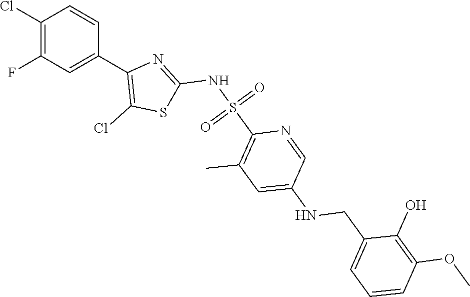 [SMILES: COc1cccc(CNc2cnc(S(=O)(=O)Nc3nc(-c4ccc(Cl)c(F)c4)c(Cl)s3)c(C)c2)c1O] | ¹H NMR (400 MHz, DMSO-d6) δ = 8.82 (s, 1H), 7.77-7.69 (m, 3H), 7.58 (dd, J = 1.6, 8.6 Hz, 1H), 7.02 (br t, J = 5.6 Hz, 1H), 6.88-6.82 (m, 2H), 6.79-6.75 (m, 1H), 6.73-6.68 (m, 1H), 4.26 (br d, J = 5.4 Hz, 2H), 3.79 (s, 3H), 2.44 (s, 3H); 19F NMR (377 MHz, DMSO-d6) δ = −115.18 (br s, 1F) |
| I-34 | 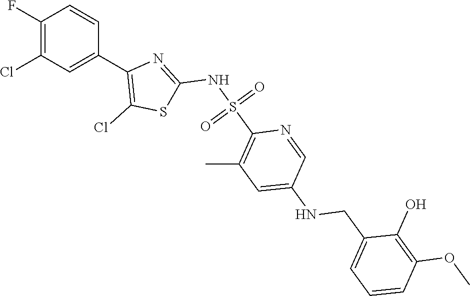 [SMILES: COc1cccc(CNc2cnc(S(=O)(=O)Nc3nc(-c4ccc(F)c(Cl)c4)c(Cl)s3)c(C)c2)c1O] | ¹H NMR (400 MHz, DMSO-d6) δ = 8.82 (s, 1H), 7.90 (dd, J = 2.2, 6.9 Hz, 1H), 7.80-7.68 (m, 2H), 7.63- 7.52 (m, 1H), 7.06-6.96 (m, 1H), 6.89-6.82 (m,2H), 6.80-6.76 (m, 1H), 6.75-6.69 (m, 1H), 4.27 (br d, J = 5.1 Hz, 2H), 3.80 (s, 3H), 2.45 (s, 3H); ¹⁹F NMR (376 MHz, CHLOROFORM-d) δ = −113.42 (br s, 1F) |
| I-35 | 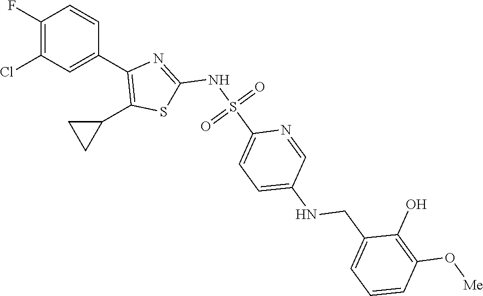 [SMILES: COc1cccc(CNc2ccc(S(=O)(=O)Nc3nc(-c4ccc(F)c(Cl)c4)c(C4CC4)s3)nc2)c1O] | ¹H NMR (400 MHz, DMSO-d6) δ = 12.90-12.47 (m, 1H), 8.82 (s, 1H), 7.95 (d, J = 2.5 Hz, 1H), 7.82 (dd, J = 2.1, 7.1 Hz, 1H), 7.69-7.58 (m, 2H), 7.57-7.46 (m, 1H), 7.12-7.01 (m, 1H), 6.91 (dd, J = 2.6, 8.8 Hz, 1H), 6.88-6.83 (m, 1H), 6.79- 6.74 (m, 1H), 6.74-6.67 (m, 1H), 4.27 (br d, J = 5.8 Hz, 2H),3.79 (s, 3H), 2.04-1.94 (m, 1H), 1.02- 0.88 (m, 2H), 0.60-0.47 (m, 2H); ¹⁹F NMR (377 MHz, DMSO-d6) δ = −113.12-−118.37 (m, 1F) |
| I-36 | 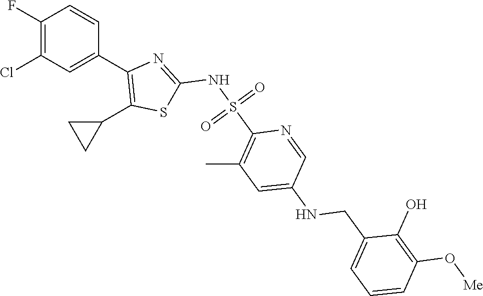 [SMILES: COc1cccc(CNc2cnc(S(=O)(=O)Nc3nc(-c4ccc(F)c(Cl)c4)c(C4CC4)s3)c(C)c2)c1O] | ¹H NMR (400 MHZ, DMSO-d6) δ = 8.80 (s, 1H), 8.13 (s, 0.1H), 7.87- 7.79 (m, 1H), 7.73-7.69 (m, 1H), 7.68-7.62 (m, 1H), 7.56-7.45 (m, 1H), 6.88-6.81 (m, 2H), 6.77 (br d, J = 7.3 Hz, 2H), 6.74-6.67 (m, 1H), 4.25 (br d, J = 5.8 Hz, 2H), 3.79 (s, 3H), 2.43 (s, 3H), 2.03-1.91 (m, 1H), 0.99-0.84 (m, 2H), 0.59-0.40 (m, 2H); ¹⁹F NMR (377 MHz, DMSO-d6) δ = −114.33-−118.78 (m, 1F) |
| I-37 | 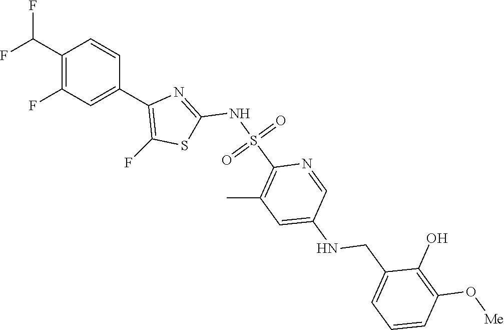 [SMILES: COc1cccc(CNc2cnc(S(=O)(=O)Nc3nc(-c4ccc(C(F)F)c(F)c4)c(F)s3)c(C)c2)c1O] | ¹H NMR (400 MHz, DMSO-d6) δ = 8.85 (brs, 1H), 7.78-7.71 (m, 2H), 7.67-7.57 (m, 2H), 7.39-7.05 (m, 2H), 6.85 (brs, 2H), 6.80-6.75 (m, 1H), 6.74-6.68 (m, 1H), 4.27 (br d, J = 2.9 Hz, 2H), 3.79 (s, 3H), 2.46 (s, 3H); 19F NMR (376 MHz, DMSO- d6) δ = −113.36 (brs, 1F), −113.51 (br s, 1F), −117.33 (brs, 1F), −148.93 (brs, 1F) |
| I-38 | 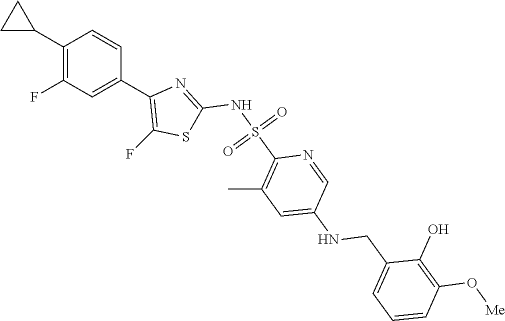 [SMILES: COc1cccc(CNc2cnc(S(=O)(=O)Nc3nc(-c4ccc(C5CC5)c(F)c4)c(F)s3)c(C)c2)c1O] | ¹H NMR (400 MHz, DMSO-d6) d 8.84 (s, 1H), 7.73 (s, 1H), 7.38 (br d, J = 9.9 Hz, 2H), 7.08 (t, J = 8.1 Hz, 1H), 7.00 (br s, 1H), 6.87-6.80 (m, 2H), 6.79-6.74 (m, 1H), 6.74-6.67 (m, 1H), 4.26 (br d, J = 5.5 Hz, 2H), 3.78 (s, 3H), 2.44 (s, 3H), 2.09- 2.05 (m, 1H), 1.04-0.96 (m, 2H), 0.79-0.72 (m, 2H); 19F NMR (376 MHz, DMSO-d6) d −119.70 (br s, 1F), −152.29 (br s, 1F) |
| I-39 | 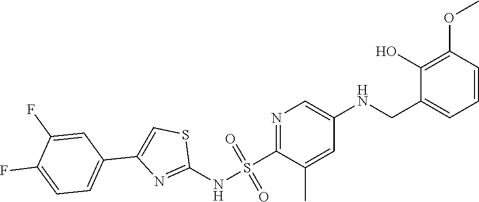 [SMILES: COc1cccc(CNc2cnc(S(=O)(=O)Nc3nc(-c4ccc(F)c(F)c4)cs3)c(C)c2)c1O] | ¹H NMR (400 MHz, DMSO-d6) δ = 8.80 (s, 1H), 7.89-7.78 (m, 1H), 7.70 (br s, 1H), 7.59 (br s, 1H), 7.56- 7.44 (m, 1H), 7.23 (br s, 1H), 6.92- 6.65 (m, 5H), 4.25 (br d, J = 5.4 Hz, 2H), 3.78 (s, 3H), 2.44 (s, 3H); 19F NMR (376 MHz, DMSO-d6) δ = −134.52-−142.20 (m, 2F) |
| I-40 | 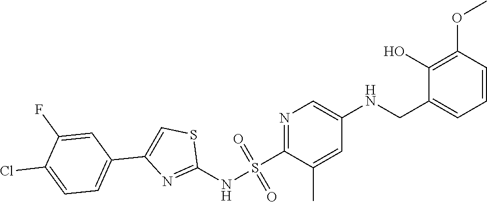 [SMILES: COc1cccc(CNc2cnc(S(=O)(=O)Nc3nc(-c4ccc(Cl)c(F)c4)cs3)c(C)c2)c1O] | ¹H NMR (400 MHz, DMSO-d6) δ = 8.80 (s, 1H), 7.86-7.78 (m, 1H), 7.74-7.59 (m, 3H), 7.42-7.31 (m, 1H), 6.97-6.88 (m, 1H), 6.85 (dd, J = 1.3, 7.9 Hz, 1H), 6.82-6.74 (m, 2H), 6.74-6.67 (m, 1H), 4.25 (br d, J = 5.9 Hz, 2H), 3.78 (s, 3H), 2.45 (s, 3H); 19F NMR (376 MHz, DMSO-d6) δ = −115.45 (br s, 1F) |
| I-41 | 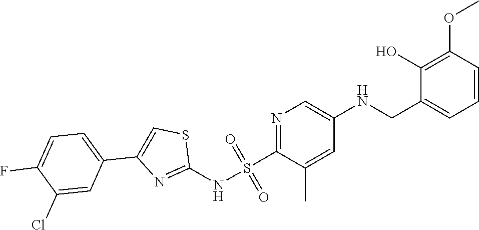 [SMILES: COc1cccc(CNc2cnc(S(=O)(=O)Nc3nc(-c4ccc(F)c(Cl)c4)cs3)c(C)c2)c1O] | ¹H NMR (400 MHz, DMSO-d6) δ = 8.81 (s, 1H), 8.03-7.95 (m, 1H), 7.83-7.67 (m, 2H), 7.49 (br t, J = 9.0 Hz, 1H), 7.35-7.23 (m, 1H), 6.97-6.63 (m, 5H), 4.25 (br d, J = 5.6 Hz, 2H), 3.78 (s, 3H), 2.45 (s, 3H); 19F NMR (377 MHz, DMSO- d6) δ = −111.9 -−119.99 (m, 1F) |
| I-42 | 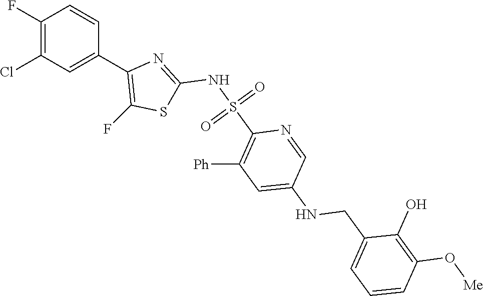 [SMILES: COc1cccc(CNc2cnc(S(=O)(=O)Nc3nc(-c4ccc(F)c(Cl)c4)c(F)s3)c(-c3ccccc3)c2)c1O] | ¹H NMR (400 MHz, DMSO-d6) δ = 13.12-11.92 (m, 1H), 8.82 (s, 1H), 7.92 (br s, 1H), 7.82 (br d, J = 7.1 Hz, 1H), 7.68-7.64 (m, 1H), 7.55 (br t, J = 9.0 Hz, 1H), 7.34 (s, 5H), 7.15 (br s, 1H), 6.86 (br d, J = 7.6 Hz, 1H), 6.80-6.77 (m, 1H), 6.75-6.68 (m, 2H), 4.29 (br d, J = 5.3 Hz, 2H), 3.78 (s, 3H); 19F NMR (376 MHz, DMSO-d6) δ = −13.36-−116.18 (m, 1F), −152.51 (br d, J = 65.4 Hz, 1F) |
| I-43 | 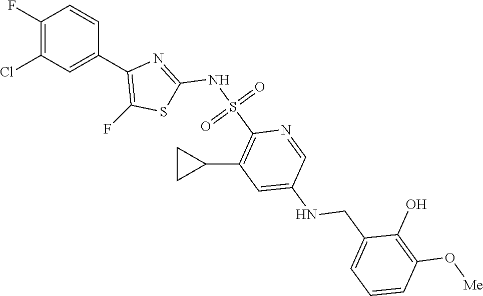 [SMILES: COc1cccc(CNc2cnc(S(=O)(=O)Nc3nc(-c4ccc(F)c(Cl)c4)c(F)s3)c(C3CC3)c2)c1O] | ¹H NMR (400 MHz, DMSO-d6) δ = 8.86 (s, 1H), 7.83 (br d, J = 7.0 Hz, 1H), 7.70-7.62 (m, 2H), 7.60-7.51 (m, 1H), 6.98 (br s, 1H), 6.85 (br d, J = 7.9 Hz, 1H), 6.78-6.74 (m, 1H), 6.73-6.66 (m, 1H), 6.43 (s, 1H), 4.24 (br d, J = 5.0 Hz, 2H), 3.79 (s, 3H), 2.64-2.59 (m, 1H), 0.99 (br d, J = 8.2 Hz, 2H), 0.66 (br d, J = 4.9 Hz, 2H); 19F NMR (376 MHz, DMSO- d6) δ = −115.20 (br d, J = 139.0 Hz, 1F), −151.93 (br s, 1F) |
| I-44 | 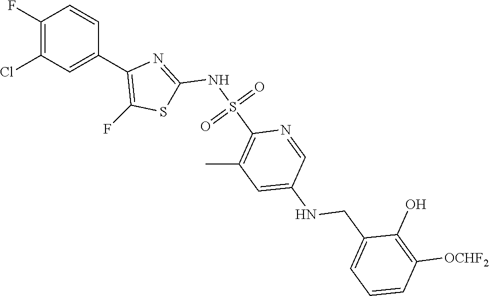 [SMILES: Cc1cc(NCc2cccc(OC(F)F)c2O)cnc1S(=O)(=O)Nc1nc(-c2ccc(F)c(Cl)c2)c(F)s1] | ¹H NMR (400 MHz, DMSO-d6): 9.50 (s, 1H), 7.86-7.72 (m, 2H), 7.69- 7.61 (m, 1H), 7.58-7.49 (m, 1H), 7.25-6.87 (m, 4H), 6.86-6.75 (m, 2H), 4.31 (br, d, J = 5.1 Hz, 2H), 2.46 (s, 3H).; 19F NMR (377 MHz, DMSO- d6): −81.34 (s, 2F), −115.25-−115.98 (m, 1F), −152.04 (br d, J = 22.6 Hz, 1F). |
| I-45 | 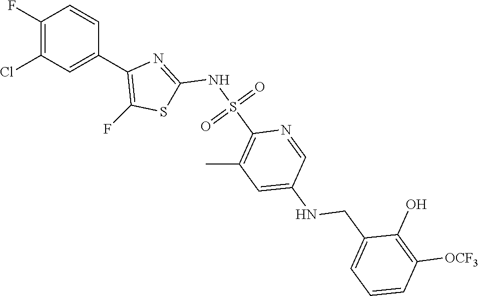 [SMILES: Cc1cc(NCc2cccc(OC(F)(F)F)c2O)cnc1S(=O)(=O)Nc1nc(-c2ccc(F)c(Cl)c2)c(F)s1] | ¹H NMR (400 MHz, DMSO-d6) δ = 9.94 (s, 1H), 7.85-7.73 (m, 2H), 7.69- 7.62 (m, 1H), 7.58-7.49 (m, 1H), 7.18 (d, J = 7.8 Hz, 2H), 7.13- .03 (m, 1H), 6.89-6.76 (m, 2H), 4.33 (d, J = 5.9 Hz, 2H), 2.46 (s, 3H); ¹⁹F NMR (377 MHz, DMSO-d6) δ = −56.92 (s, 3F), −115.56-−117.23 (m, 1F), −152.08-−152.40 (m, 1F) |
| I-46 | 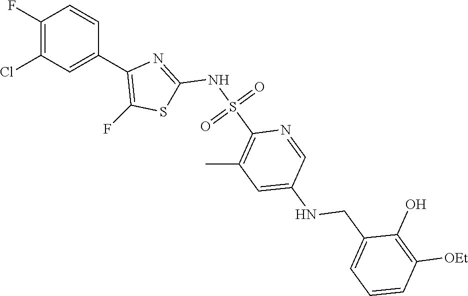 [SMILES: CCOc1cccc(CNc2cnc(S(=O)(=O)Nc3nc(-c4ccc(F)c(Cl)c4)c(F)s3)c(C)c2)c1O] | ¹H NMR (400 MHz, DMSO-d6) δ = 8.61 (br s, 1H), 7.85-7.73 (m, 2H), 7.70-7.61 (m, 1H), 7.58-7.47 (m, 1H), 7.09-6.99 (m, 1H), 6.90-6.80 (m, 2H), 6.76 (br d, J = 7.4 Hz, 1H), 6.72-6.63 (m, 1H), 4.27 (br s, 2H), 4.08-3.98 (m, 2H), 2.45 (s, 3H), 2.07 (d, J = 3.5 Hz, 1H), 1.37-1.30 (m, 3H); 19F NMR (377 MHz, DMSO-d6) δ = −115.52 (br s, 1F), −151.95 (brs, 1F) |
| I-47 | 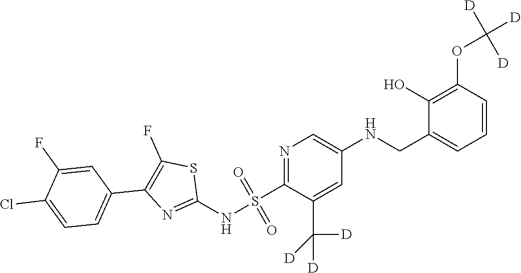 [SMILES: [2H]C([2H])([2H])Oc1cccc(CNc2cnc(S(=O)(=O)Nc3nc(-c4ccc(Cl)c(F)c4)c(F)s3)c(C([2H])([2H])[2H])c2)c1O] | ¹H NMR (400 MHz, DMSO-d6) δ = 8.83 (s, 1H), 7.75-7.65 (m, 2H), 7.62 (br d, J= 10.9 Hz, 1H), 7.52 (br d, J = 8.4 Hz, 1H), 6.97 (br s, 1H), 6.85 (br d, J = 7.7 Hz, 1H), 6.81 (br s, 1H), 6.79-6.75 (m, 1H), 6.74-6.67 (m, 1H), 4.26 (brd, J = 5.6 Hz, 2H), 3.79 (s, 3H); |
| I-48 | 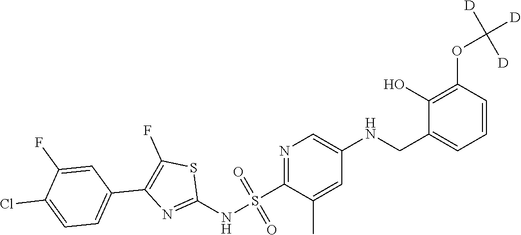 [SMILES: [2H]C([2H])([2H])Oc1cccc(CNc2cnc(S(=O)(=O)Nc3nc(-c4ccc(Cl)c(F)c4)c(F)s3)c(C)c2)c1O] | ¹H NMR (400 MHz, DMSO-d6) δ = 8.99-8.68 (m, 1H), 7.78-7.66 (m, 2H), 7.63 (dd, J = 1.8, 10.7 Hz, 1H), 7.52 (br d, J = 8.0 Hz, 1H), 6.87- 6.81 (m, 2H), 6.79-6.67 (m, 2H), 4.26 (s, 2H), 2.45 (s, 3H); 19F NMR (377 MHz, DMSO-d6): −115.04 (brs, 1F), −149.93 (br s, 1F); 19F NMR (376 MHz, DMSO-d6) δ = −113.97- −116.18 (m, 1F), −149.06 (br d, J = 179.8 Hz, 1F) |
| I-49 | 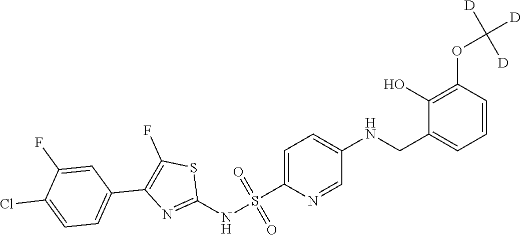 [SMILES: [2H]C([2H])([2H])Oc1cccc(CNc2ccc(S(=O)(=O)Nc3nc(-c4ccc(Cl)c(F)c4)c(F)s3)nc2)c1O] | ¹H NMR (400 MHz, DMSO-d6) δ = 8.85 (br s, 1H), 7.97 (br s, 1H), 7.73- 7.58 (m, 3H), 7.51 (br s, 1H), 7.19 (br s, 1H), 6.95-6.82 (m, 2H), 6.79- 6.66 (m, 2H), 4.27 (br s, 2H); ¹⁹F NMR (376 MHz, DMSO-d6) δ −113.56-−116.99 (m, 1F) |
| I-50 | 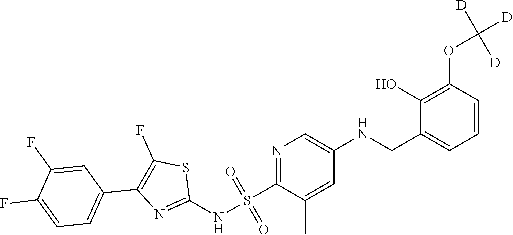 [SMILES: [2H]C([2H])([2H])Oc1cccc(CNc2cnc(S(=O)(=O)Nc3nc(-c4ccc(F)c(F)c4)c(F)s3)c(C)c2)c1O] | ¹H NMR (400 MHz, DMSO-d6) δ = 8.83 (br s, 1H), 7.75 (br s, 1H), 7.71- 7.62 (m, 1H), 7.61-7.47 (m, 2H), 6.89-6.81 (m, 2H), 6.79-6.74 (m, 1H), 6.74-6.66 (m, 1H), 4.26 (br s, 2H), 2.45 (s, 3H); 19F NMR (376 MHz, DMSO-d6) δ d −73.84- −75.37 (m, 1F) |
| I-51 | 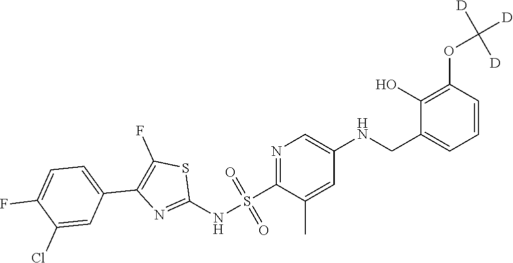 [SMILES: [2H]C([2H])([2H])Oc1cccc(CNc2cnc(S(=O)(=O)Nc3nc(-c4ccc(F)c(Cl)c4)c(F)s3)c(C)c2)c1O] | ¹H NMR (400 MHz, DMSO-d6) δ = 8.78 (s, 1H), 7.79 (dd, J = 1.8, 7.1 Hz, 1H), 7.82-7.76 (m, 1H), 7.71-7.62 (m, 2H), 7.47 (br t, J = 8.7 Hz, 1H), 6.86-6.73 (m, 4H), 6.72-6.67 (m, 1H), 4.24 (br d, J = 5.9 Hz, 2H), 2.43 (s, 3H); ¹⁹F NMR (376 MHz, DMSO- d6) δ = d −74.05 (br s, 1F) |

In some embodiments, the present invention provides a compound as depicted in Table 1 or pharmaceutically acceptable salts thereof.

### Compositions and Formulations

### Parenteral Dosage Forms.

In one embodiment, the novel compounds described herein can be administered in a parenteral dosage form. The term “parenteral,” as used herein, includes, but is not limited to, subcutaneous injections, intravenous, intramuscular, intraperitoneal injections, or infusion techniques. In an embodiment, the parenteral pharmaceutical formulations described herein comprises a compound described herein and a pharmaceutically acceptable carrier. In one embodiment, the parenteral pharmaceutical formulation may contain liquid carriers, including, vegetable oils such as peanut oil, cotton seed oil, sesame oil, as well as organic solvents, PEG, propylene glycol, glycerol, and surfactants. In certain embodiments for parenteral administration, the conjugates of the invention are mixed with solubilizing agents such as Cremophor™, alcohols, oils, modified oils, glycols, polysorbates, cyclodextrins, polymers, and mixtures thereof.

In another embodiment, the parenteral pharmaceutical formulation is aqueous. In one embodiment, the aqueous parenteral pharmaceutical formulation comprises at least 50% water, preferably 70% or more of water. In another embodiment, the pharmaceutical carrier comprises a solubilizer. In a specific embodiment, the solubilizer is 2-hydroxyalkylated β-cyclodextrin such as hydroxyethyl β-CD, hydroxypropyl-β-CD, hydroxybutyl β-CD, or sulfobutylether-β-cyclodextrin.

The pharmaceutically acceptable 2-hydroxyalkylated β-cyclodextrin may be present in any suitable amount within the aqueous parenteral pharmaceutical formulations described herein. The pharmaceutically acceptable 2-hydroxyalkylated β-cyclodextrin may be present in amount of between about 5% and 50% w/v. For example, the pharmaceutically acceptable solubilizer, optionally HP-β-CD, may be in an amount of about 10%, 15%, 20%, 25%, 30%, 35%, 40%, 45%, or 50% w/v. In specific embodiments, the HP-β-CD may be present in amount from about 10% to about 40%, from about 20% to about 40%, from about 30% to about 40%, from about 10% to about 30%, or from about 10% to about 20% of the total formulation. In one embodiment, the 2-hydroxyalkylated β-cyclodextrin may present in amount of about 25% of the total formulation w/v.

For parenteral administration, sterile solutions and suspensions are desired. Isotonic preparations which generally contain suitable preservatives are employed when intravenous, administration is desired. The pharmaceutical formulations may be administered parenterally via injection of a pharmaceutical formulation comprising a compound dissolved in an inert liquid carrier, such as sterile water or other pharmaceutically acceptable diluents.

Pharmaceutical compositions or formulations suitable for parenteral administration include aqueous and non-aqueous sterile injection solutions which may contain antioxidants, buffers, bacteriostats and solutes that render the formulation isotonic with the blood of the intended recipient; and aqueous and non-aqueous sterile suspensions which may include suspending agents and thickening agents. The compositions may be presented in unit-dose or multi-dose containers, sealed ampules and vials, and may be stored in a freeze-dried (lyophilized) condition requiring only the addition of the sterile liquid carrier, e.g., water for injections, saline, dextrose in water (D5W), lactated Ringer's solution, immediately prior to use. Extemporaneous immediately prior to use. Extemporaneous injection solutions and suspensions may be prepared from sterile powders, granules and tablets of the kind previously described.

Parenteral formulations may further comprise at least one of any suitable auxiliaries including, but not limited to, diluents, crystal inhibitors, tonicifiers, water structure forming agents or disruptors, polymers, ion pairing agents, stabilizers, buffers, salts, lipophilic solvents, preservatives, adjuvants or the like. Pharmaceutically acceptable auxiliaries are preferred. Examples and methods of preparing such sterile solutions are well known in the art and can be found in well-known texts such as, but not limited to, REMINGTON'S PHARMACEUTICAL SCIENCES (Adejare, Ed., 123rd Edition, Academic Press. (2020); Handbook of Pharmaceutical Excipients, 9^{th} Edition, Pharmaceutical Press (2020)). Pharmaceutically acceptable carriers can be routinely selected that are suitable for the mode of administration, solubility and/or stability of the compound.

Pharmaceutical excipients and additives useful in the parenteral pharmaceutical formulations described herein can also include, but are not limited to, proteins, peptides, amino acids, lipids, and carbohydrates (e.g., sugars, including monosaccharides, di-, tri-, tetra-, and oligosaccharides; derivatized sugars such as alditols, aldonic acids, esterified sugars; and polysaccharides or sugar polymers), which can be present singly or in combination, comprising alone or in combination in ranges of 1-99.99% by weight or volume. Exemplary protein excipients include serum albumin such as human serum albumin (HSA), recombinant human albumin (rHA), gelatin, and casein. Representative amino acid components, which can also function in a buffering capacity, include alanine, glycine, arginine, betaine, histidine, glutamic acid, aspartic acid, cysteine, lysine, leucine, isoleucine, valine, methionine, phenylalanine, and aspartame.

Carbohydrate excipients suitable for use in the parenteral pharmaceutical formulations described herein include but are not limited to monosaccharides such as dextrose, fructose, maltose, galactose, glucose, D-mannose, and sorbose; disaccharides, such as lactose, sucrose, trehalose, and cellobiose; polysaccharides, such as raffinose, melezitose, maltodextrins, dextrans, and starches; and alditols, such as mannitol, xylitol, maltitol, lactitol, xylitol, sorbitol (glucitol), and myoinositol.

The pharmaceutical formulations comprising the compounds described herein can also a pH adjusting agent. The pH adjusting agent may be a base, optionally sodium hydroxide. The pH adjusting agent may also be an acid, optionally hydrochloric acid.

In one embodiment, the present disclosure provides stable parenteral pharmaceutical formulations as well as preserved solutions and formulations containing a preservative, as well as multi-use preserved formulations suitable for pharmaceutical or veterinary use, comprising at least one compound disclosed herein in a pharmaceutically acceptable formulation. Pharmaceutical formulations in accordance with the present disclosure may optionally comprise at least one known preservative. Preservatives include, but are not limited to, phenol, m-cresol, p-cresol, o-cresol, chlorocresol, benzyl alcohol, phenylmercuric nitrite, phenoxyethanol, formaldehyde, chlorobutanol, magnesium chloride (e.g., hexahydrate), alkylparaben (methyl, ethyl, propyl, butyl), benzalkonium chloride, benzethonium chloride, sodium dehydroacetate and thimerosal, or mixtures thereof in an aqueous diluent. Any suitable concentration or mixture can be used as known in the art, such as 0.001-5%, or any range or value therein. Non-limiting examples include, no preservative, 0.1-2% m-cresol, 0.1-3% benzyl alcohol, 0.001-0.5% thimerosal, 0.001-2.0% phenol, 0.0005-1.0% alkylparaben(s).

Other excipients, e.g., isotonicity agents, buffers, antioxidants, preservative enhancers, can be optionally added. When used, the amount of tonicity modifier used most often ranges from 0.1 to 1% (w/v). Non-limiting examples of suitable tonicity modifiers include sodium chloride, glycerin, boric acid, calcium chloride, dextrose, and potassium chloride. The formulations can include a local anesthetic to reduce the potential of pain during injection. A physiologically tolerated buffer can be added to provide improved pH control if necessary. The pharmaceutical formulations can cover a wide range of pHs, such as from about pH 4 to about pH 10, specifically, a range from about pH 5 to about pH 9, and more specifically, a range of about 7.0 to about 9.0. In one aspect, the formulations described herein have pH between about 8.4 and about 8.7.

Methods of preparing the pharmaceutical preparations described herein are manufactured in a manner that is known, including conventional mixing, dissolving, or lyophilizing processes. Thus, aqueous pharmaceutical preparations can be obtained by combining the active compounds with solid excipients, optionally grinding the resulting mixture and processing the mixture of granules, after adding suitable auxiliaries, if desired or necessary.

### Oral Dosage Forms

Pharmaceutical formulation of the compounds described herein for oral administration may be in the form of tablets or capsules and may be immediate-release formulations or may be controlled- or extended-release formulations, which may contain pharmaceutically acceptable excipients, such as corn starch, mannitol, povidone, magnesium stearate, talc, cellulose, methylcellulose, carboxymethylcellulose and similar substances. A pharmaceutical composition comprising the compounds and/or a salt thereof may comprise one or more pharmaceutically acceptable excipients, which are known in the art. Formulations include oral films, orally disintegrating tablets, effervescent tablets and granules or beads that can be sprinkled on food or mixed with liquid as a slurry or poured directly into the mouth to be washed down.

Pharmaceutical compositions containing the compounds, salts and hydrates thereof can be prepared by any method known in the art of pharmaceutics. In general, such preparatory methods include the steps of bringing the 12-LOX inhibitor or a pharmaceutically acceptable salt thereof into association with a carrier or excipient, and/or one or more other accessory ingredients, and then, if necessary and/or desirable, shaping, and/or packaging the product into a desired single- or multi-dose unit.

Pharmaceutical compositions can be prepared, packaged, and/or sold in bulk, as a single unit dose, and/or as a plurality of single unit doses. As used herein, a “unit dose” is a discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

Relative amounts of the active ingredient, the pharmaceutically acceptable excipient, and/or any additional ingredients in a pharmaceutical composition of the invention will vary, depending upon the identity, size, and/or condition of the subject treated and further depending upon the route by which the composition is to be administered. The composition used in the methods of the present invention may comprise between 0.001% and 100% (w/w) active ingredient.

Pharmaceutically acceptable excipients used in the manufacture of provided pharmaceutical compositions include inert diluents, dispersing and/or granulating agents, surface active agents and/or emulsifiers, disintegrating agents, binding agents, preservatives, buffering agents, lubricating agents, and/or oils. Excipients such as cocoa butter and suppository waxes, coloring agents, coating agents, sweetening, flavoring, and perfuming agents may also be present in the composition.

In certain embodiments, the pharmaceutical composition used in the methods of the present invention may comprise a diluent. Exemplary diluents include calcium carbonate, sodium carbonate, calcium phosphate, dicalcium phosphate, calcium sulfate, calcium hydrogen phosphate, sodium phosphate, lactose, sucrose, cellulose, microcrystalline cellulose, kaolin, mannitol, sorbitol, inositol, sodium chloride, dry starch, cornstarch, powdered sugar, and mixtures thereof.

In certain embodiments, the pharmaceutical composition used in the methods of the present invention may comprise a granulating and/or dispersing agent. Exemplary granulating and/or dispersing agents include potato starch, corn starch, tapioca starch, sodium starch glycolate, clays, alginic acid, guar gum, citrus pulp, agar, bentonite, cellulose, and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (VEEGUM), sodium lauryl sulfate, quaternary ammonium compounds, and mixtures thereof.

In certain embodiments, the pharmaceutical composition used in the methods of the present invention may comprise a binding agent. Exemplary binding agents include starch (e.g., cornstarch and starch paste), gelatin, sugars (e.g., sucrose, glucose, dextrose, dextrin, molasses, lactose, lactitol, mannitol, etc.), natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (VEEGUM.RTM.), and larch arabogalactan), alginates, polyethylene oxide, polyethylene glycol, inorganic calcium salts, silicic acid, polymethacrylates, waxes, water, alcohol, and/or mixtures thereof.

In certain embodiments, the pharmaceutical composition used in the methods of the present invention may comprise a preservative. Exemplary preservatives include antioxidants, chelating agents, antimicrobial preservatives, antifungal preservatives, antiprotozoan preservatives, alcohol preservatives, acidic preservatives, and other preservatives. In certain embodiments, the preservative is an antioxidant. In other embodiments, the preservative is a chelating agent.

In certain embodiments, the pharmaceutical composition used in the methods of the present invention may comprise an antioxidant. Exemplary antioxidants include alpha tocopherol, ascorbic acid, ascorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

In certain embodiments, the pharmaceutical composition used in the methods of the present invention may comprise a chelating agent. Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

In certain embodiments, the pharmaceutical composition may comprise a buffering agent together with the 12-LOX inhibitor or the salt thereof. Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, and mixtures thereof.

In certain embodiments, the pharmaceutical composition used in the methods of the present invention may comprise a lubricating agent. Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, and mixtures thereof.

In other embodiments, the pharmaceutical composition of containing a 12-LOX inhibitor or salt thereof will be administered as a liquid oral dosage form. Liquid dosage forms for oral and parenteral administration include pharmaceutically acceptable emulsions, microemulsions, solutions, suspensions, syrups, and elixirs. In addition to the active ingredients, the liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (e.g., cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can include adjuvants such as wetting agents, emulsifying and suspending agents, sweetening, flavoring, and perfuming agents. In certain embodiments for parenteral administration, the conjugates of the invention are mixed with solubilizing agents such as Cremophor™, alcohols, oils, modified oils, glycols, polysorbates, cyclodextrins, polymers, and mixtures thereof.

Solid dosage forms for oral administration include capsules, tablets, pills, powders, and granules. In such solid dosage forms, the active ingredient is mixed with at least one inert, pharmaceutically acceptable excipient or carrier such as sodium citrate or dicalcium phosphate and/or (a) fillers or extenders such as starches, lactose, sucrose, glucose, mannitol, and silicic acid, (b) binders such as, for example, carboxymethylcellulose, alginates, gelatin, polyvinylpyrrolidinone, sucrose, and acacia, (c) humectants such as glycerol, (d) disintegrating agents such as agar, calcium carbonate, potato or tapioca starch, alginic acid, certain silicates, and sodium carbonate, (e) solution retarding agents such as paraffin, (f) absorption accelerators such as quaternary ammonium compounds, (g) wetting agents such as, for example, cetyl alcohol and glycerol monostearate, (h) absorbents such as kaolin and bentonite clay, and (i) lubricants such as talc, calcium stearate, magnesium stearate, solid polyethylene glycols, sodium lauryl sulfate, and mixtures thereof. In the case of capsules, tablets, and pills, the dosage form may include a buffering agent.

Some oral compositions of the invention relate to extended- or controlled-release formulations. These may be, for example, diffusion-controlled products, dissolution-controlled products, erosion products, osmotic pump systems or ionic resin systems. Diffusion-controlled products comprise a water-insoluble polymer which controls the flow of water and the subsequent egress of dissolved drug from the dosage from. Dissolution-controlled products control the rate of dissolution of the drug by using a polymer that slowly solubilizes or by microencapsulation of the drug—using varying thicknesses to control release. Erosion products control release of drug by the erosion rate of a carrier matrix. Osmotic pump systems release a drug based on the constant inflow of water across a semi permeable membrane into a reservoir which contains an osmotic agent. Ion exchange resins can be used to bind drugs such that, when ingested, the release of drug is determined by the ionic environment within the gastrointestinal tract.

### Administration and Dosage

One of ordinary skill in the art will appreciate that administration of pharmaceutically effective amounts of the pharmaceutical formulations described herein to a patient in need thereof, can be determined empirically, or by standards currently recognized in the medical arts. It will be understood that, when administered to a human patient, the total daily usage of the agents of the compositions described herein will be decided within the scope of sound medical judgment by the attending physician. The specific therapeutically effective dose level for any particular patient will depend upon a variety of factors: the type and degree of the cellular response to be achieved; activity of the specific agent or composition employed; the specific agents or composition employed; the age, body weight, general health, gender and diet of the patient; the time of administration, route of administration, and rate of excretion of the agent; the duration of the treatment; drugs used in combination or coincidental with the specific agent; and like factors well known in the medical arts. It is well within the skill of the art to start doses of the agents at levels lower than those required to achieve the desired therapeutic effect and to gradually increase the dosages until the desired effect is achieved.

Routes of administration and dosages of effective amounts of parenteral pharmaceutical formulations comprising the 12-LOX inhibitor compounds are also disclosed. In one embodiment, the administration is intravenous. The pharmaceutical compositions and/or formulations described herein may also be administered by infusion. The pharmaceutical formulations described herein may also be administered by a bolus dosage, optionally combined with administration by infusion. The compounds described herein can be administered in combination with other pharmaceutical agents in a variety of protocols for effective treatment of disease.

The parenteral pharmaceutical formulations may be administered in a single daily dose, or the total daily dosage may be administered in divided doses of two, three, or four times daily. Doses maybe administered for one week, one month, or over the course of several months, 3, 6, 9 or 12 months, or intervals known in the art and determined to be clinically relevant. In one embodiment, the parenteral pharmaceutical formulations may be administered chronically for years or for the life of the patient. For example, the pharmaceutical formulations described herein may be administered for 7 days, 14 days, 21 days, 28 days, or 35 days. The daily dosage of the formulations may be varied over a wide range from about 0.0001 to about 1,000 mg per patient, per day, more particularly from about 200 to about 700, preferably about 500 to 600 mg/day. The range may more particularly be from about 1 mg/kg to 100 mg/kg of body weight per day, about 10-50 mg/kg, 20 to 60 mg/kg, preferably 10-20 mg/kg per day for adults (at about 60 kg), or equivalent doses as determine by a practitioner, to achieve a serum concentration that is clinically relevant.

Specifically, the aqueous parenteral pharmaceutical formulations described herein may be administered at least once a day over the course of several weeks, several months, or several years. In one embodiment, the pharmaceutical formulations are administered at least once a day over several weeks to several months. In another embodiment, the pharmaceutical formulations are administered once a day over at least one year.

The aqueous parenteral pharmaceutical formulations may be administered following use of a topical anesthetic including but not limited to lidocaine, prilocaine, or combinations thereof. Ice or ethyl chloride spray can also be used prior to administration

It can be sometimes desirable to deliver the compounds described herein to the subject over prolonged periods of time, for periods of one week to one year from a single administration. Certain medical devices may be employed to provide a continuous intermittent or on demand dosing of a patient. The devices may be a pump of diffusion apparatus, or other device containing a reservoir of drug and optionally diagnostic or monitoring components to regulate the delivery of the drug. Various slow-release, depot or implant dosage forms can be utilized. These may be, for example, diffusion-controlled products, dissolution-controlled products, erosion products, osmotic pump systems or ionic resin systems. Diffusion-controlled products comprise a water-insoluble polymer which controls the flow of water and the subsequent egress of dissolved drug from the dosage from. Dissolution-controlled products control the rate of dissolution of the drug by using a polymer that slowly solubilizes or by microencapsulation of the drug—using varying thicknesses to control release. Erosion products control release of drug by the erosion rate of a carrier matrix.

In one embodiment, the aqueous parenteral pharmaceutical formulations described herein may be administered by infusion. The infusion may comprise infusing between about 1 and about 1000 mg of the novel compounds in the aqueous pharmaceutical formulation continuously. In another embodiment, the infusion is administered over 30 minutes to 23 hours.

The amount of the compound infused may be about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1000 mg.

In one embodiment, the infusion may be continuous or repeated over intervals. In one embodiment, the intervals are between about 1 day to 1 year, or between about 1 day to 6 months, or 1 day to 1 month. In another embodiment, the infusion may be repeated over between about 1 and 14 days, optionally between about 1 and 7 days. The infusion may be repeated over 1, 2, 3, 4, 5, 6, 7, 8, 9, 10, 11, 12, 13, or 14 days. The infusion may be repeated over between about 1 and 7 days, 3 and 8 days, 5 and 14 days, 9 and 14 days, or 7 and 14 days.

For example, the pharmaceutical formulations described herein may be infused continuously or over a period of 1 to 23 hours, including all intervals in between, at a rate of about 10 mg, 20 mg, 30 mg, 40 mg, 50 mg, 60 mg, 70 mg, 80 mg, 90 mg, 100 mg, 110 mg, 120 mg, 130 mg, 140 mg, 150 mg, 160 mg, 170 mg, 180, 190 mg, 200 mg, 210 mg, 220 mg, 230 mg, 240 mg, 250 mg, 260 mg, 270 mg over 1-5 hours, 280 mg, 290 mg, 300 mg, 310 mg, 320 mg, 330 mg, 340 mg, 350 mg, 360 mg, 370 mg, 380 mg, 390 mg, 400 mg, 510 mg, 520 mg, 530 mg, 540 mg, 550 mg, 560 mg, 570 mg, 580 mg, 590 mg, 600 mg, 610 mg, 620 mg, 630 mg, 640 mg, 650 mg, 660 mg, 670 mg, 680 mg, 690 mg, 700 mg, 710 mg, 720 mg, 730 mg, 740 mg, 750 mg, 760 mg, 770 mg, 780 mg, 790 mg, 800 mg, 810 mg, 820 mg, 830 mg, 840 mg, 850 mg, 860 mg, 870 mg, 880 mg, 890 mg, 900 mg, 910 mg, 920 mg, 930 mg, 940 mg, 950 mg, 960 mg, 970 mg, 980 mg, 990 mg, or 1000 mg. In a specific embodiment, infusion occurs over 1 to 12 hours, 1 to 6 hours, or 1 to 4 hours.

For oral dosage forms or formulations, the pharmaceutical compositions may be administered in a single daily dose, or the total daily dosage may be administered in divided doses of two, three, or four times daily. Doses maybe administered for one week, one month, or over the course of several months, 3, 6, 9 or 12 months, chronically, or intervals known in the art and determined to be clinically relevant. Doses may be continued throughout the life of the patient, or discontinues when clinical judgment warrants. The daily dosage of the compositions may be varied over a wide range from about 0.0001 to about 1,000 mg per patient, per day. The range may more particularly be from about 0.001 mg/kg to 10 mg/kg of body weight per day, about 0.1-100 mg, about 1. 0-50 mg or about 1.0-0 mg per day for adults (at about 60 kg). Additionally, the dosages may be about 0.5-10 mg/kg, per day, about 1.0-5.0 mg/kg per day, 5.0-10 mg/kg per day, or equivalent doses as determine by a practitioner, to achieve a plasma/serum concentration that is clinically relevant.

The aqueous pharmaceutical formulations described herein can be administered to any animal that can experience the beneficial effects of the compounds of the invention. Preferred is administration to humans.

The dosage administered will be dependent upon the age, health, and weight of the recipient, kind of concurrent treatment, if any, frequency of treatment, and the nature of the effect.

### Therapeutic and Prophylactic Methods

The novel 2-aminothiazole compounds and other compounds described herein may be used as selective 12-lipoxygenase (12-LOX) inhibitors and can be used to prevent or treat 12-LOX-mediated diseases. The disclosure further provides a method for treating or preventing a 12-LOX mediated disease or disorder comprising administering to a mammal a therapeutically or prophylactically effective amount of a novel 2-aminothiazole derived compound or other compounds described herein.

Lipoxygenases are involved in the first committed step in a cascade of metabolic pathways and the products of these enzymes (eicosanoids) are precursors of hormones such as leukotrienes and lipoxins, which mediate a wide array of cellular functions. (Serhan, et al. Chem. Rev. 2011, 111, 5922-5943). Consequently, the lipoxygenase enzymes and their bioactive metabolites (e.g. hydroxyeicosatetraenoic acid (HETE) and leukotriene A4) have been implicated in a variety of inflammatory diseases and cancers.

12-LOX has been demonstrated to play a role in a number of conditions and/or diseases, such as skin diseases and platelet hemostasis, transplantation/xenotransplantation, cancer (including but not limited to prostate cancer, colorectal cancer, breast cancer, lung cancer, and hematological diseases), Type 1 and Type 2 diabetes, diabetic kidney disease (diabetic nephropathy), diabetic neuropathy, diabetic retinopathy, cardiovascular disease (including but not limited to myocardial infarction, congestive heart failure, heart failure, and stroke), thrombosis, heparin induced thrombocytopenia (HIT), Alzheimer's disease, non-alcoholic steatohepatitis, non-alcoholic fatty liver disease, insulin resistance, arthritis, lupus (SLE), lupus nephritis, and inflammation.

In one embodiment, the invention provides a method for reducing U46619-induced, thrombin-induced, PAR1-AP, PAR4-AP-induced or collagen-induced platelet aggregation, comprising administering to a mammal thereof a therapeutically or prophylactically effective amount of any of compounds, salts, prodrugs, enantiomers, a mixture of enantiomers, or diastereomer thereof.

In one embodiment, the invention provides a method for reducing FcγRIIA-mediated platelet activation (e.g via anti-CD9 or IV.3+GAM-induced activation) comprising administering to a mammal thereof a therapeutically or prophylactically effective amount of any of the compounds, salts, prodrugs, enantiomers, mixture of enantiomers, or diastereomer thereof.

In certain embodiments, the disorder is an immune-mediated thrombocytopenia and thrombosis disorder including but not limited to thrombocytopenia associated with sepsis, and heparin-induced thrombocytopenia (HIT).

In preferred embodiments, the disease or disorder to be treated by the disclosed 12-LOX inhibitor compounds is selected from thrombosis, HIT, and Type 1 and Type 2 diabetes.

In a specific embodiment, the disorder is heparin-induced thrombocytopenia (HIT).

In a specific embodiment, the disorder is Type 1 diabetes.

In one embodiment, the invention provides method of treating or preventing a 12-lipoxygenase mediated disease or disorder, comprising administering to a mammal thereof a therapeutically or prophylactically effective amount of any of compounds or a salt, prodrugs, enantiomers, a mixture of enantiomers, or diastereomer thereof.

### Combination Therapy

The pharmaceutical formulations described herein can also include additional therapeutic agents or combinations thereof.

An additional therapeutic agent may be other 12-LOX inhibitors. A 12-LOX inhibitor can be an organic compound, an inorganic compound, a biological compound (e.g., proteins or fragments thereof, antibodies or fragments thereof, nucleic acids, nucleic acid analogs, saccharides, or peptides), or any combination thereof. A 12-LOX inhibitor can also be synthetic or naturally occurring. Selective 12-LOX inhibitors are described in U.S. Pat. Nos. 10,266,488 and 10,752,581.

The mixing ratios of the 12-LOX inhibitors may be optimized to provide maximum therapeutic effects.

Additional agents include but not limited to anti-thrombotic agents such as argatroban, fondaparinux, lepirudin, bivalirudin, danaparoid and drotrecogin alfa; antidiabetic agents such as exenatide, albiglutide, pramlintide, semaglutide, lixisenatide, and dulaglutide. Combinations with direct-acting oral coagulants is also contemplated, including but not limited to apixaban, dabigatran, rivaroxaban, and edoxaban.

The invention also contemplates co-administration of the foregoing additional therapeutic agents in separate compositions or formulations, either at the same time, before or after administration of the 12-LOX inhibitor compounds of the disclosure.

### Biological Examples

The compounds were evaluated for inhibition of 12-LOX, as well as selectivity by measuring inhibition against 15-LOX for some compounds, using a UV-Vis assay. Arachidonic acid (AA) was used as a substrate for 12-LOX and 15-LOX. ML355 was used as the control inhibitor for 12-LOX. ML351 (5-(Methylamino)-2-(1-naphthalenyl)-4-oxazolecarbonitrile) was used as the control inhibitor for 15-LOX.

Reaction buffers used for the lipoxygenase assay were as follows: 25 mM HEPES (pH 7.5), with 0.01% Triton X-100. 1 mM 12-HPETE or 1 mM 15-HPETE stock solution was prepared in 25 mM HEPES with 0.01% Triton X-100.

The standard curve of 12-HPETE was made by serial dilution of 0, 6.25, 12.5, 25, 50, 75 and 100 μM, and the final reaction volume in 96 Well Clear Flat Bottom UV-Transparent Microplate is 100 μL. The absorbance of the 12-HPETE at each concentration was measured at 234 nm.

The standard curve of 15-HPETE was made by serial dilution of 0, 3.125, 6.25, 12.5, 25, 50 and 100 μM, and the final reaction volume in 96 Well Clear Flat Bottom UV-Transparent Microplate is 100 μL. The absorbance of the 12-HPETE at each concentration was measured at 234 nm.

IC₅₀ values were obtained by determining the % inhibition at various inhibitor concentrations. The final concentrations of the control inhibitors and test compounds in the IC₅₀ determination assay are 0, 0.03, 0.1, 0.3, 1, 3, 10 and 20 μM. The final concentration of DMSO in the assay is 0.05%.

12-LOX IC₅₀ determination. The 12-LOX enzyme and AA were diluted in the HEPES buffer to 120 nM and 200 μM, respectively. Pre-incubation 50 μL of indicated test compounds and 25 μL 120 nM enzyme at room temperature for 5 min. The reaction is started by adding 25 μL 200 μM AA. After a short spin (1000 rpm, 15 s), incubate the reaction system for 5 min. The blank control was set by adding 25 μL HEPES to 50 μL of control inhibitor compound (MHL355) and 25 μL 200 μM AA. The final concentrations of 12-LOX enzyme and AA were 30 nM and 50 μM, respectively. The absorbance of the 12-HPETE at 234 nm was measured.

15-LOX IC₅₀ determination. The 15-LOX enzyme and AA were diluted in the HEPES buffer to 480 nM and 200 μM, respectively. Pre-incubation 50 μL of indicated test compounds and 25 μL 480 nM enzyme at room temperature for 5 min. The reaction is started by adding 25 μL 200 μM AA. After a short spin (1000 rpm, 15 s), incubate the reaction system for 3 min. The blank control was set by adding 25 μL HEPES to 50 μL of control inhibitor compound (MHL355) and 25 μL 200 μM AA. The final concentrations of 15-LOX enzyme and AA were 120 nM and 50 M, respectively. The absorbance of the 15-HPETE at 234 nm was measured. The assay was performed in duplicate.

The IC₅₀ value was calculated by using GraphPad Prism 8.0.2.

The IC₅₀ values are calculated with remaining activity (%) and logarithm of inhibitor concentrations.

Remaining Activity (%)=OD value inhibitor/OD value vehicle×100

The IC₅₀ values for the compounds are in Table 2 below:

TABLE 2

| Compound | IC₅₀ 12-LOX | Selectivity |
|---|---|---|
| No.: | (μM) | over 15-LOX |
| I-1 | +++ | >50-fold |
| I-2 | +++ | >50-fold |
| I-3 | +++ | >20-fold |
| I-4 | +++ | >50-fold |
| I-5 | +++ | >50-fold |
| I-6 | +++ | >50-fold |
| I-7 | +++ | >50-fold |
| I-8 | +++ | >50-fold |
| I-9 | ++ | >50-fold |
| I-10 | + | ND |
| I-11 | ++ | >20-fold |
| I-12 | +++ | >50-fold |
| I-13 | +++ | >50-fold |
| I-14 | ++ | ~10-fold |
| I-15 | + | <10-fold |
| I-16 | +++ | >50-fold |
| I-17 | +++ | >20-fold |
| I-18 | +++ | >50-fold |
| I-19 | + | ND |
| I-20 | ND | ND |
| I-21 | ND | ND |
| I-22 | + | <10-fold |
| I-23 | +++ | >50-fold |
| I-24 | +++ | >50-fold |
| I-25 | +++ | >50-fold |
| I-26 | ND | ND |
| I-27 | ND | ND |
| I-28 | ND | ND |
| I-29 | ND | ND |
| I-30 | ND | ND |
| I-31 | ND | ND |
| I-32 | ND | ND |
| I-33 | ND | ND |
| I-34 | ND | ND |
| I-35 | ND | ND |
| I-36 | ND | ND |
| I-37 | ND | ND |
| I-38 | ND | ND |
| I-39 | +++ | >50-fold |
| I-40 | +++ | >50-fold |
| I-41 | +++ | >50-fold |
| I-42 | +++ | >50-fold |
| I-43 | + | <20-fold |
| I-44 | ND | ND |
| I-45 | + | <20-fold |
| I-46 | + | <20-fold |
| I-47 | + | <20-fold |
| I-48 | +++ | >50-fold |
| I-49 | +++ | >50-fold |
| I-50 | ++ | >50-fold |
| I-51 | +++ | >50-fold |

+++ = <250 nM
++ = 250 nM-1000 nM
+ = >1000 nM

The foregoing merely summarizes certain aspects of this disclosure and is not intended, nor should it be construed, as limiting the disclosure in any way.

### INCORPORATION BY REFERENCE

The entire disclosure of each of the patent documents and scientific articles referred to herein is incorporated by reference for all purposes.

### EQUIVALENTS

The present disclosure may be embodied in other specific forms without departing from the spirit or essential characteristics thereof. The foregoing embodiments are therefore to be considered in all respects illustrative rather than limiting the invention described herein. The scope of the disclosure is thus indicated by the appended claims rather than by the foregoing description, and all changes that come within the meaning and range of equivalency of the claims are intended to be embraced therein.

What is claimed is:

1. A compound of formula (I): 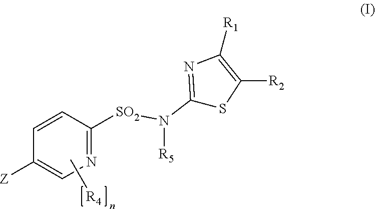
wherein Z is 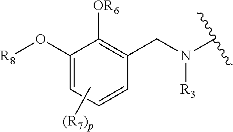
and wherein
R₁ is selected from hydrogen or optionally substituted cycloalkyl, aryl or C₁-C₃ alkylaryl, each of which is optionally substituted by one or more substituents independently selected from halogen, alkyl, haloalkyl, alkyloxy, C₁-C₃ haloalkyloxy or heterocycle;
R₂ is selected from H, halogen, deuterium, alkyl, haloalkyl, C₁-C₃ alkyloxy and C₁-C₃ haloalkyloxy, hydroxyalkyl, alkyoxyalkyl, cycloalkyl including cyclopropyl, C₁-C₃ alkylaryl, or substituted or unsubstituted aryl and aralkyl including 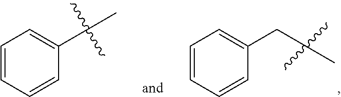 [SMILES: *c1ccccc1 ; *Cc1ccccc1]
or amide including 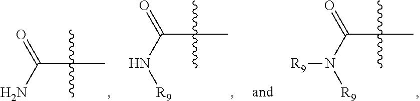
wherein R₉ is independently selected from C₁-C₃ alkyl, cycloalkyl (including cyclopropyl), and substituted or unsubstituted phenyl;
or R₁ and R₂ together form an optionally substituted 5, 6, or 7-membered fused 1,2-carbocyclic ring system with the thiazole ring wherein the ring carbons of the 5, 6, or 7-membered fused ring are optionally substituted by one or more atoms selected from N, O, and S. The fused 5, 6, or 7-membered ring is optionally substituted independently by one or more halogen, C₁-C₃ alkyl, C₁-C₃ haloalkyl, C₁-C₃ alkyloxy, C₁-C₃ haloalkyloxy, or an optionally substituted aryl ring;
or R₁ and R₂ together with the carbons to which they are attached form substituted or unsubstituted 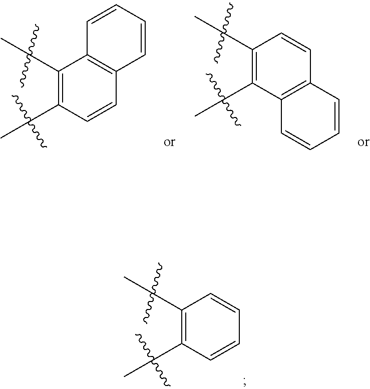 [SMILES: *c1ccc2ccccc2c1* ; *c1ccc2ccccc2c1* ; *c1ccccc1*]
R₃ is hydrogen, C₁-C₃ alkyl, or C₁-C₃ haloalkyl;
each R₄ is independently hydrogen, CD₃, substituted or unsubstituted phenyl, cycloalkyl including cyclopropyl, C₁-C₃ alkyl, C₁-C₃ haloalkyl, —OR, C₁-C₃ haloalkyloxy, or halogen;
R₅ is hydrogen, C₁-C₃ alkyl, or C₁-C₃ haloalkyl;
R₆ is hydrogen, CD3, alkyl, cycloalkyl, haloalkyl or 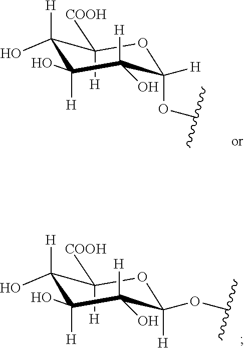
each R₇ is independently hydrogen, alkyl, haloalkyl, cycloalkyl, or halogen;
R₈ is hydrogen, alkyl, haloalkyl, cycloalkyl, or halocycloalkyl;
n is 0, 1, or 2;
p is 0, 1, 2, or 3; or
a pharmaceutically acceptable salt, prodrug, solvate, hydrate, or stereoisomer thereof.

2. The compound of claim 1, wherein the compound is formula (I).

3. The compound according to claim 1 wherein R₁ is phenyl substituted by two or more substituents independently selected from halogen, alkyl, haloalkyl, 3-F, 4-oxetanephenyl, and C₁-C₃ haloalkyloxy.

4. The compound of any one of claims 1-3, wherein R₁ is phenyl substituted by one or more halogen.

5. The compound of any one of claims 1-4, wherein R₁ is

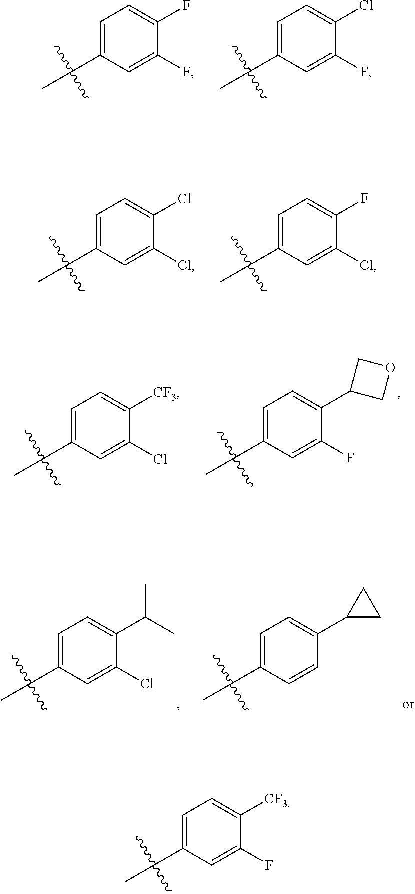

6. The compound of any one of claims 1-5, wherein R₂ is hydrogen or deuterium.

7. The compound of any one of claims 1-6, wherein R₂ is halogen.

8. The compound of any one of claims 1-7, wherein R₂ is fluorine or chlorine.

9. The compound of any one of claims 1-5, wherein wherein R₂ is selected from substituted or unsubstituted aryl or aralkyl, hydroxyalkyl, cyclopropyl, or amide.

10. A composition comprising the compound of formula (I) or a pharmaceutically acceptable salt, prodrug, solvate, hydrate, or stereoisomer thereof, according to claim 1, and a pharmaceutically acceptable carrier.

11. A method of treating or preventing a 12-lipoxygenase-mediated disease and/or disorder comprising administering to the subject a therapeutically effective amount of the compound of formula (I), a pharmaceutically acceptable salt, prodrug, solvate, hydrate, or stereoisomer thereof, according to claim 1.

12. The compound 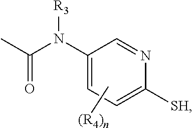
or a salt thereof, wherein R₃, R₄, and n are defined in claim 1.

13. The compound of formula (II): 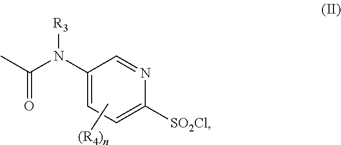
or a pharmaceutically acceptable salt thereof, wherein R₃, R₄ and n are defined in claim 1.

14. The compound according to formula IV 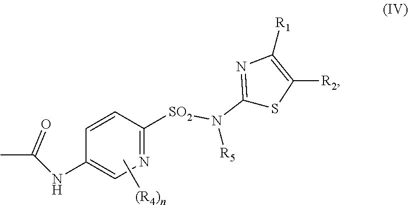
or a salt thereof, wherein R₁, R₂, R₄, R₅ and n are defined in claim 1.

15. The compound according to formula V: 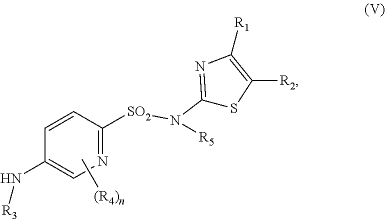
or a pharmaceutically acceptable salt thereof, wherein R₁ to R₅ and n are defined in claim 1.

16. A compound of formula (VII) or (VIIa): 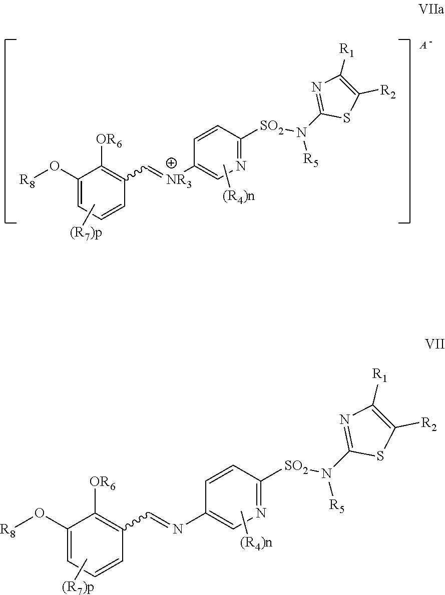
or a pharmaceutically acceptable salt thereof, wherein R₁ to R₈, n, and p are defined in claim 1.

17. The compound according to claim 1, wherein R₄ is methyl, fluorine, or CF₃.

18. The compound according to claim 4, wherein R₄ is methyl, fluorine, or CF₃.
